(19) United States
(12) Patent Application Publication
(10) Pub. No.: US 2024/0293416 A1
(43) Pub. Date: Sep. 5, 2024

(54) TREATMENT OF LIVER DISORDERS WITH A THR-ß AGONIST

(71) Applicant: Terns Pharmaceuticals, Inc., Foster City, CA (US)

(72) Inventors: Christopher T. JONES, Foster City, CA (US); Kevin KLUCHER, Foster City, CA (US); D. Barry CRITTENDEN, Foster City, CA (US); Erin K. QUIRK, Foster City, CA (US); Feng JIN, Foster City, CA (US); Matt DUAN, Foster City, CA (US); Sheng GUO, Foster City, CA (US); Jianwei BIAN, Foster, City, CA (US); Qianqian DONG, Foster City, CA (US)

(21) Appl. No.: 18/651,843

(22) Filed: May 1, 2024

Related U.S. Application Data
(63) Continuation of application No. PCT/CN2022/131297, filed on Nov. 11, 2022.

(30) Foreign Application Priority Data
Nov. 11, 2021 (WO) PCT/CN2021/130083
Jun. 7, 2022 (WO) PCT/CN2022/097426

(51) Int. Cl. A61K 31/53; A61K 9/00; A61P 1/16; C07D 403/12

(52) U.S. Cl. CPC A61K 31/53; A61K 9/0053; A61P 1/16; C07D 403/12

(57) ABSTRACT

Provided herein are methods for treating liver disorders, including non-alcoholic steatohepatitis, and symptoms and manifestations thereof, in a patient which utilize treatment with a THR-β agonist.

### CROSS REFERENCE TO RELATED APPLICATIONS

This application is a continuation of International Application No. PCT/CN2022/131297, filed on Nov. 11, 2022, which claims the benefit of and priority to International Application No. PCT/CN2021/130083, filed on Nov. 11, 2021, and International Application No. PCT/CN2022/097426 filed on Jun. 7, 2022. The entire contents of the aforementioned patent applications are incorporated herein by reference in their entirety for all purposes.

### FIELD OF THE INVENTION

This invention relates to methods and compositions for treating liver disorder in a patient.

### BACKGROUND

Fatty liver disease (FLD) encompasses a spectrum of disease states characterized by excessive accumulation of fat in the liver often accompanied with inflammation. FLD can lead to non-alcoholic fatty liver disease (NAFLD), which may be characterized by insulin resistance. If untreated, NAFLD can progress to a persistent inflammatory response or non-alcoholic steatohepatitis (NASH), progressive liver fibrosis, and eventually to cirrhosis. In Europe and the US, NAFLD is the second most common reason for liver transplantation. Accordingly, the need for treatment is urgent, but due to the lack of obvious symptoms to the patient, patients may lack the motivation to maintain treatment regimens, particularly burdensome treatment regimens, such as injected medicines, medications that are administered many times a day, or any that produce dangerous or irritating side effects. There is currently no approved treatment of NASH.

Thyroid hormone receptor-beta (THR-β) agonists have recently been investigated in the treatment of liver disease, including NASH. THR-β is the major form of THR in the liver and plays a key role in energy balance and metabolism of fatty acids and lipids, whereas THR-α predominates in the heart and is responsible for most of the unwanted cardiovascular effects of thyroid hormone stimulation. A significant problem to overcome involves developing a THR-β agonist for treating NASH that will not produce undesirable side effects associated with THR-α agonism.

### BRIEF SUMMARY

Provided herein are methods and compositions for treating a liver disorder in a patient in need thereof. The methods comprise administering to the patient the thyroid hormone receptor beta (THR-β) agonist referred to herein as Compound 1, or a pharmaceutically acceptable salt thereof.

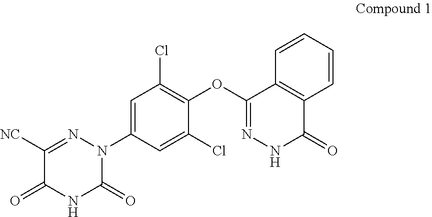 [SMILES: N#Cc1nn(-c2cc(Cl)c(Oc3n[nH]c(=O)c4ccccc34)c(Cl)c2)c(=O)[nH]c1=O]

Compound 1, which has the chemical name 2-(3,5-dichloro-4-((4-oxo-3,4-dihydrophthalazin-1-yl)oxy)phenyl)-3,5-dioxo-2,3,4,5-tetrahydro-1,2,4-triazine-6-nitrile, was described in U.S. Pat. No. 11,084,802, which is incorporated herein by reference in its entirety.

Inventors have discovered that Compound 1 or a pharmaceutically acceptable thereof, can be administered to patients suffering from liver disorders at surprisingly low doses while still maintaining the desired level of efficacy. As a result, Compound 1 can be used to treat liver disorders without the undesirable side effects generally associated with THR agonism.

In some embodiments of the disclosure, Compound 1, or a pharmaceutically acceptable salt thereof, can be administered to patients orally once daily at doses as low as 1 mg or less and still sufficiently reduce amine oxidase activity and reduce lymphocyte adhesion and transmigration. For example, Compound 1, or a pharmaceutically acceptable salt thereof, can be administered orally once daily to a patient with a liver disorder (e.g. NASH) at a dose of from about 1 mg to about 60 mg. In some embodiments, Compound 1, or a pharmaceutically acceptable salt thereof, can be administered orally once daily to a patient with a liver disorder (e.g. NASH) at a dose of from about 0.5 mg to about 25 mg. In some embodiments, Compound 1, or a pharmaceutically acceptable salt thereof, can be administered orally once daily to a patient with a liver disorder (e.g. NASH) at a dose of from about 1 mg to about 15 mg. In some embodiments, Compound 1, or a pharmaceutically acceptable salt thereof, can be administered orally once daily to a patient with a liver disorder (e.g. NASH) at a dose of from about 3 mg to about 10 mg. In some embodiments, Compound 1, or a pharmaceutically acceptable salt thereof, can be administered orally once daily to a patient with a liver disorder (e.g. NASH) at a dose of about 1 mg. In some embodiments, Compound 1, or a pharmaceutically acceptable salt thereof, can be administered orally once daily to a patient with a liver disorder (e.g. NASH) at a dose of about 3 mg. In some embodiments, Compound 1, or a pharmaceutically acceptable salt thereof, can be administered orally once daily to a patient with a liver disorder (e.g. NASH) at a dose of about 4 mg. In some embodiments, Compound 1, or a pharmaceutically acceptable salt thereof, can be administered orally once daily to a patient with a liver disorder (e.g. NASH) at a dose of about 5 mg. In some embodiments, Compound 1, or a pharmaceutically acceptable salt thereof, can be administered orally once daily to a patient with a liver disorder (e.g. NASH) at a dose of about 6 mg. In some embodiments, Compound 1, or a pharmaceutically acceptable salt thereof, can be administered orally once daily to a patient with a liver disorder (e.g. NASH) at a dose of about 10 mg. In some embodiments, Compound 1, or a pharmaceutically acceptable salt thereof, can be administered orally once daily to a patient with a liver disorder (e.g. NASH) at a dose of about 15 mg. In some embodiments, Compound 1, or a pharmaceutically acceptable salt thereof, can be administered orally once daily to a patient with a liver disorder (e.g. NASH) at a dose of about 20 mg. In some embodiments, Compound 1, or a pharmaceutically acceptable salt thereof, can be administered orally once daily to a patient with a liver disorder (e.g. NASH) at a dose of about 30 mg. In some embodiments, Compound 1, or a pharmaceutically acceptable salt thereof, can be administered orally once daily to a patient with a liver disorder (e.g. NASH) at a dose of about 50 mg. In some embodiments, Compound 1, or a pharmaceutically acceptable salt thereof, can be administered orally once daily to a patient with a liver disorder (e.g. NASH) at a dose of about 60 mg.

In another aspect, the disclosure provide methods of treating or preventing NASH in a patient in need thereof, said method comprising administering to the patient a therapeutically effective amount of Compound 1, or a pharmaceutically acceptable salt thereof. In one embodiment, the patient in need thereof is a patient that suffers from fatty liver disease such as NAFLD. In another embodiment, the patient in need thereof is a patient that suffers from metabolic syndrome.

In one aspect, the disclosure provides methods of reducing hepatic inflammation in a patient in need thereof, comprising administering to the patient a therapeutically effective amount of Compound 1, or a pharmaceutically acceptable salt thereof. The reduction of hepatic inflammation is characterized by reduced expression of inflammatory genes and markers of leukocyte activation in the liver. In some embodiments, hepatic inflammation is reduced without increasing the low-density lipoprotein cholesterol (LDL-C) levels in the blood of the patient.

In another aspect, the disclosure provides methods of treating a disease or condition characterized by fibrosis of the liver, comprising administering to the patient a therapeutically effective amount of Compound 1, or a pharmaceutically acceptable salt thereof. The reduction of fibrosis is characterized by histological improvement and reduced expression of pro-fibrotic genes in the liver. In some embodiments, hepatic fibrosis is reduced without increasing the low-density lipoprotein cholesterol (LDL-C) levels in the blood of the patient. In some embodiments, administration of Compound 1, or a pharmaceutically acceptable salt thereof, results in reduction of liver fibrosis and hepatic inflammation.

In some embodiments, the patient has a liver disorder and diabetes mellitus. In some embodiments, the patient has a liver disorder and a cardiovascular disorder. In some embodiments, the treatment period is the remaining lifespan of the patient. In some embodiments, the method does not comprise administering an antihistamine, an immunosuppressant, a steroid, rifampicin, an opioid antagonist, or a selective serotonin reuptake inhibitor (SSRI).

In some embodiments, Compound 1 is administered to the patient as a pharmaceutically acceptable salt. In some embodiments, the pharmaceutically acceptable salt is a potassium salt. In some embodiments, the pharmaceutically acceptable salt is a sodium salt.

The disclosure also provide novel compositions comprising Compound 1, or a pharmaceutically acceptable salt thereof. Compound 1 has very low aqueous solubility, even when administered in the salt form. It has been found that particular ionic surfactants can effectively solubilize Compound 1, and pharmaceutically salts thereof, with minimal or no degradation of the compound. In some embodiments, the ionic surfactant is sodium lauryl sulfate. In some such embodiments, the amount of SLS in the composition is from about 1% to about 8% by weight. In other such embodiments, the amount of SLS in the composition is about 5% by weight. In some embodiments, the pharmaceutical composition comprises the potassium salt of Compound 1 and SLS.

### BRIEF DESCRIPTION OF THE FIGURES

FIG. 1 shows the plasma concentration of compound 1 in patients up to 72 hours following administration of a single dose of compound 1.

FIG. 2 shows the area under the curve (AUC_{inf}) and maximum plasma concentration (Cₘₐₓ) of compound 1 in patients plotted against the administered dose.

FIG. 3 shows the mean percent change in sex hormone binding globulin (SHBG) 4 days after administration of a single dose of compound 1.

FIG. 4 shows the mean percent change in apolipoprotein B (Apo B) 4 days after administration of a single dose of compound 1.

FIG. 5 shows free T3, T4, and TSH on day 15 after 14 days of daily administration of compound 1 or placebo in humans.

FIG. 6 shows percent change in free testosterone, total testosterone, and sex hormone binding globulin (SHBG) from baseline on day 15 after 14 days of daily administration of compound 1 or placebo in humans.

FIG. 7 shows plasma concentrations of Compound 1 over time on days 1 and 14 of a multiple ascending dose study wherein Compound 1 was dosed once daily.

FIG. 8 shows percent change in pharmacodynamics markers (sex hormone binding globulin, ApoB, total cholesterol, LDL-c, HDL-c, and triglycerides) from baseline on day 15 after 14 days of daily administration of compound 1 or placebo in humans.

FIG. 9 shows the effects of Compound 1 on body and organ weight in mouse NASH model.

FIG. 10 shows the effects of Compound 1 on liver steatosis, inflammation, and fibrosis in mouse NASH model.

FIG. 11 shows the effects of Compound 1 on lipids and indicators of liver injury (ALT) in mouse NASH model.

FIG. 12 shows the effects of Compound 1 on expression of genes associated with collagen extracellular matrix and hepatic stellate cell activation.

FIG. 13 shows the plasma concentration of compound 1 in beagle dogs following administration of a single 50 mg dose provided in one of two oral formulations (PO1 or PO2).

FIG. 14 shows the plasma concentration of compound 1 in two groups of beagle dogs following administration of a single 10 mg dose provided in oral formulation PO3. One group was pretreated with pentagastrin, while the other group was pretreated with famotidine.

FIG. 15 shows the plasma concentration of compound 1 in fasted and fed beagle dogs following administration of a single 10 mg dose provided in oral formulation PO3.

FIG. 16 shows the XRPD spectrum of compound 1, potassium salt form, type A.

FIG. 17 shows the XRPD spectrum of compound 1, free acid form, type A.

FIG. 18 shows the XRPD spectrum of compound 1, sodium salt form, type A.

FIG. 19 shows the XRPD spectrum of compound 1, L-arginine salt form, type A.

FIG. 20 shows the XRPD spectrum of compound 1, magnesium salt form, type A.

FIG. 21 shows the overall study design for compound 1.

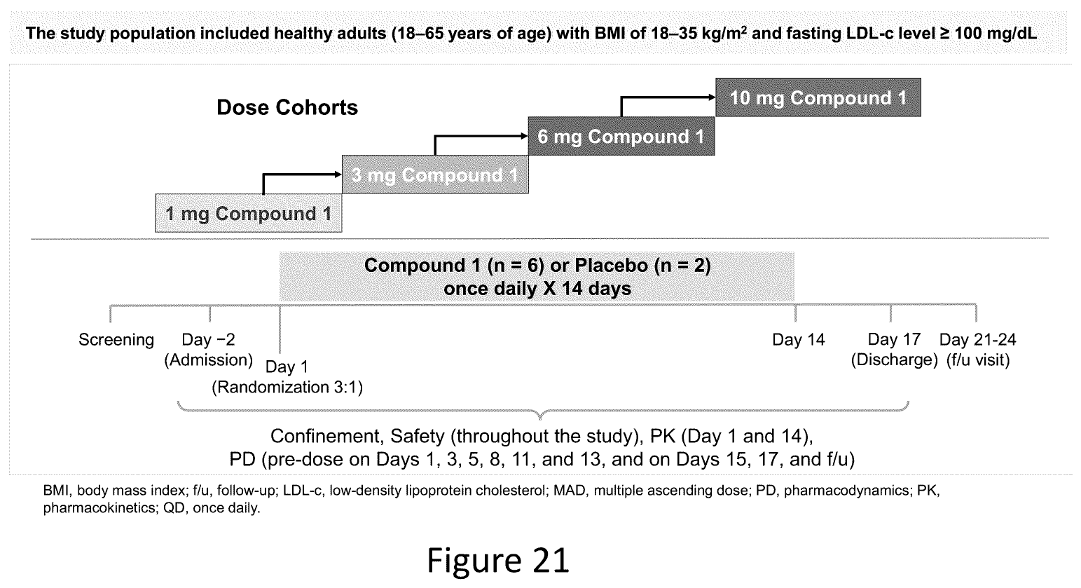

FIG. 22 shows the demographics and baseline characteristics for compound 1.

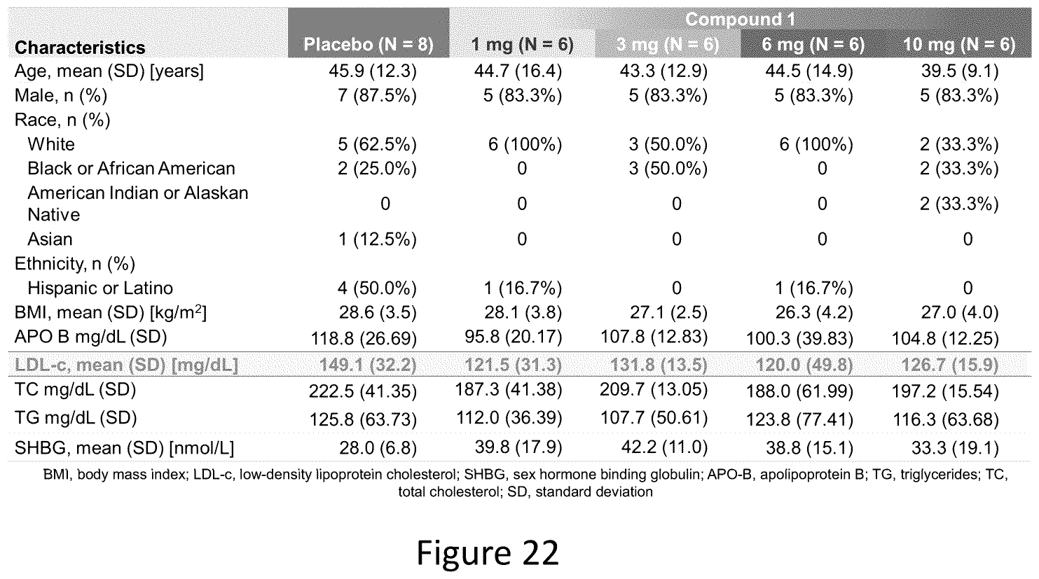

FIG. 23 shows the plasma concentration-time profile, day 14, for compound 1.

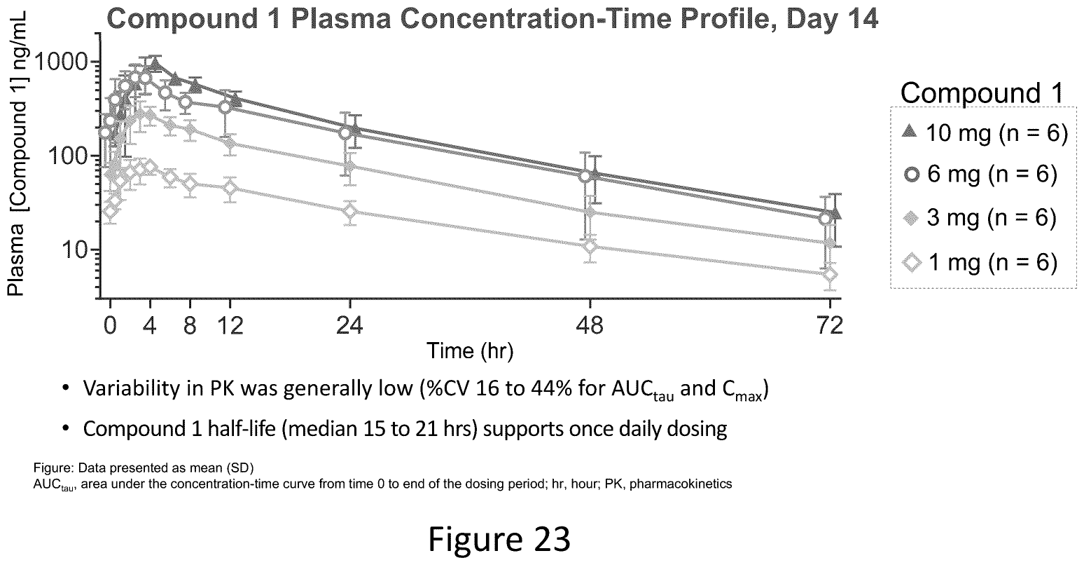

FIG. 24 shows the day 14, PK parameters of compound 1.

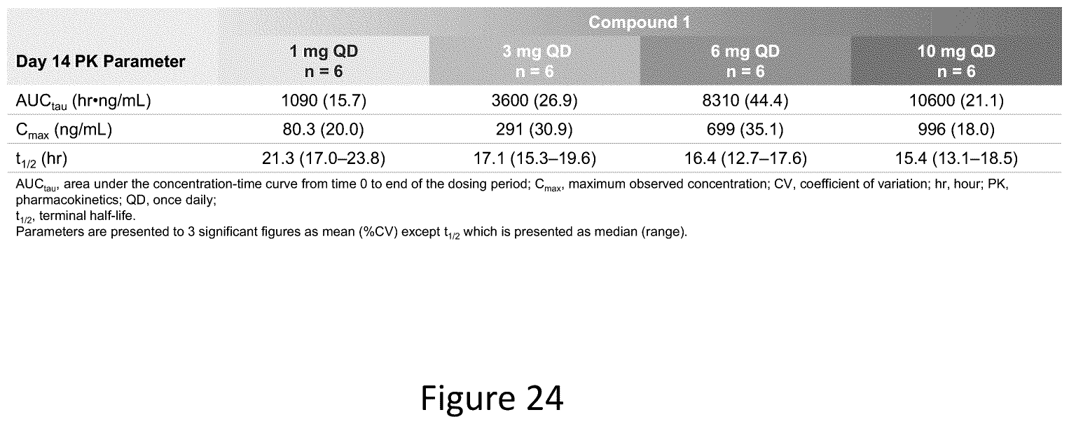

FIG. 25 shows the sex hormone binding globulin (SHBG) (percent change from baseline to Day 15).

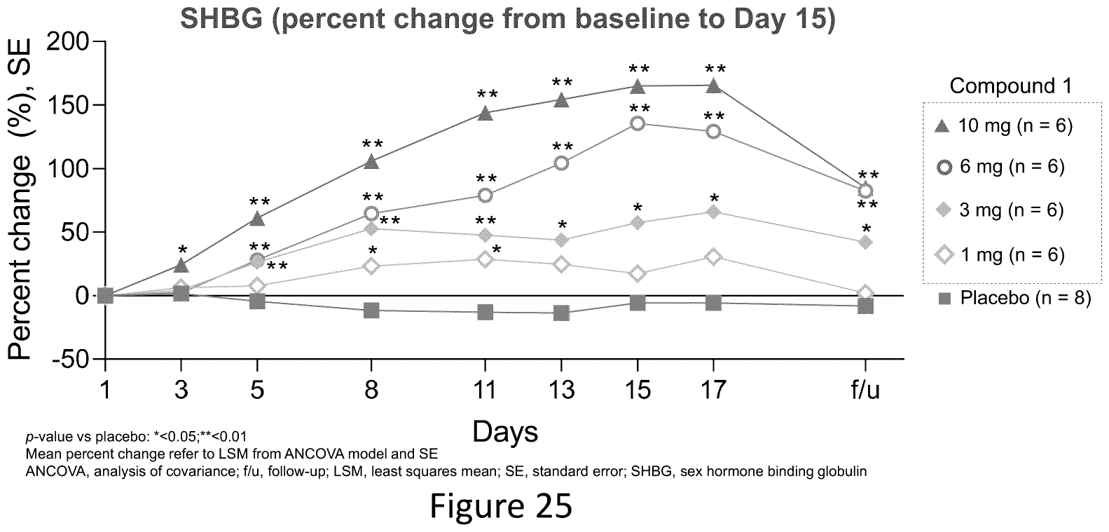

FIG. 26 shows the LDL-c (percent change from baseline to Day 15).

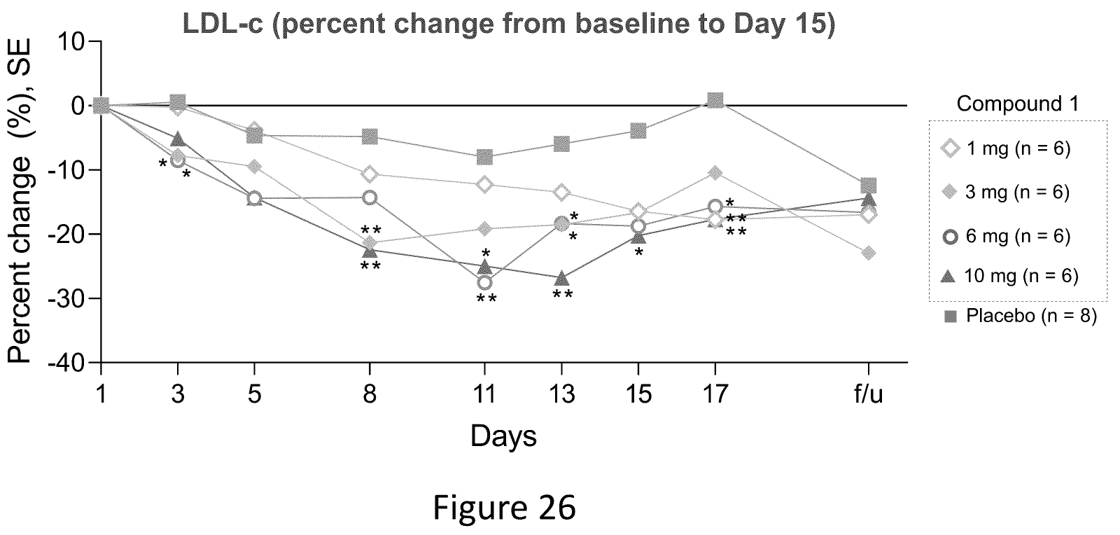

FIG. 27 shows the percent change from baseline at end of treatment (Day 15) for SHBG and LDL-c per compound 1 dose.

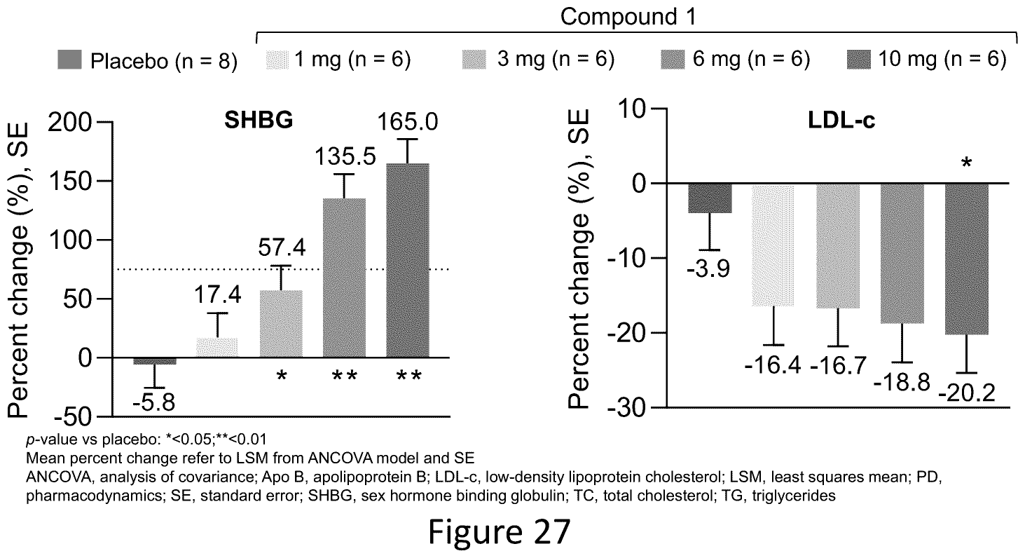

FIG. 28 shows the decreases in total cholesterol (TC), Apo B, and triglycerides (TG) per compound 1 dose.

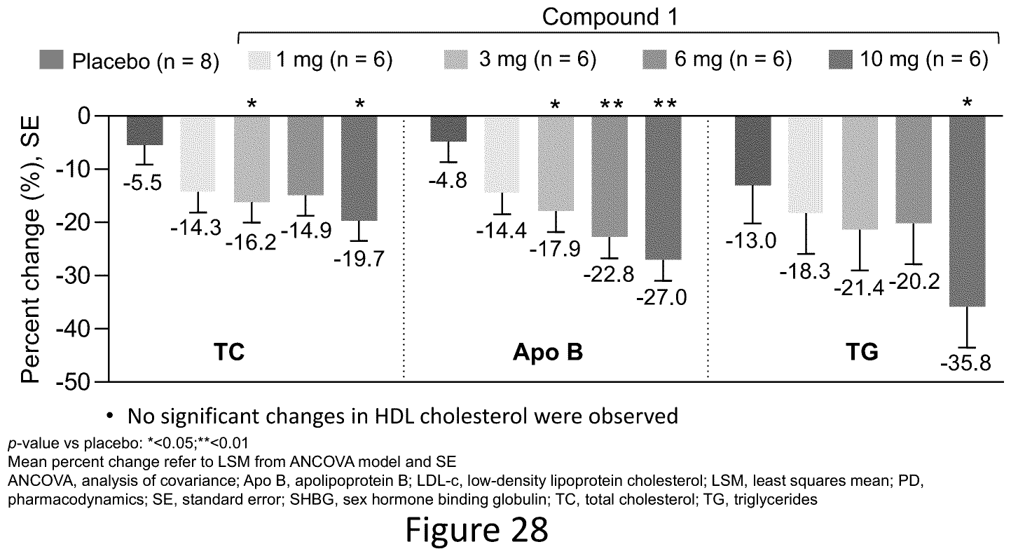

FIG. 29 shows that the treatment-emergent adverse events were mild and mostly unrelated with no significant changes in vital signs.

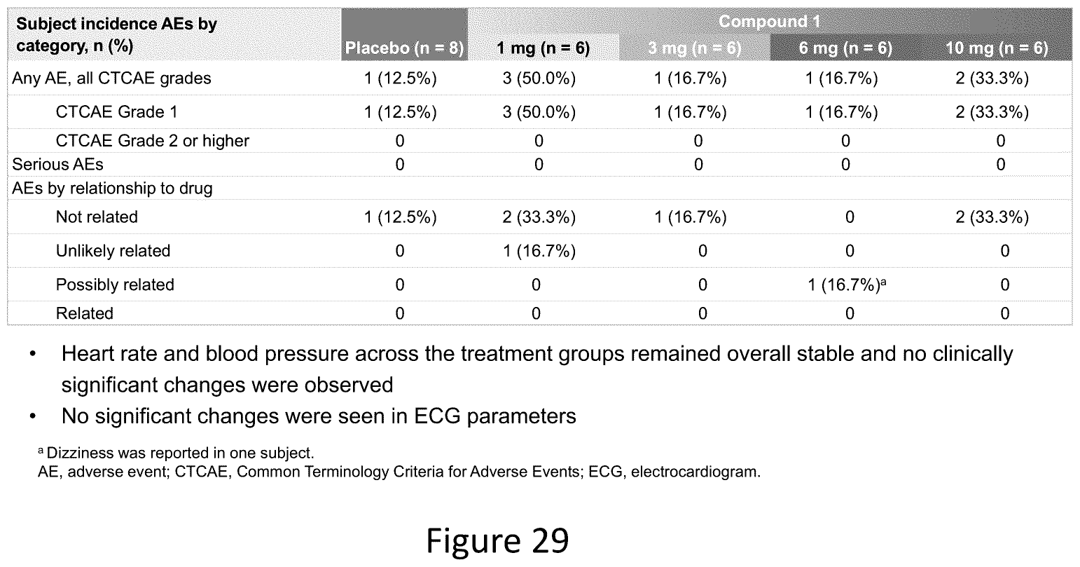

FIG. 30A and FIG. 30B show body weight change in male biopsy-confirmed DIO-NASH mice. Compound 2 alone and in combination with Compound 1 reduced body weight. Body weights were measured daily during study. Body weight change during study is shown in FIG. 30A, where dots represent mean body weight change relative to baseline (n=10-16 mice per group). In FIG. 30B, bars represent mean (SD) body weight measured on Week 11 of the study. Lean vehicle control, white; DIO-GAN vehicle control, gray; Compound 2, blue; Compound 1-low, light orange; Compound 1-med, orange; Compound 1-high, dark orange; Combo-low, light purple; Combo-med, purple; Combo-high, dark purple. Statistical comparison to DIO-GAN vehicle control determined by ANOVA followed by Tukey correction for multiple comparisons. *p<0.05; **p<0.01; ***p<0.001; ****p<0.0001.

FIG. 31 shows Discrete weekly food intake during the study. Dots represent mean weekly food intake in grams (n=10-16 mice per group). Lean vehicle control, white; DIO-GAN vehicle control, gray; Compound 2, blue; Compound 1-low, light orange; Compound 1-med, orange; Compound 1-high, dark orange; Combo-low, light purple; Combo-med, purple; Combo-high, dark purple.

FIG. 32A and FIG. 32B shows liver and spleen weight. Compound 2 and Compound 1 alone and in combination significantly reduced hepatomegaly without changes in spleen weight. Bars represent mean (SD) liver (FIG. 32A) and spleen (FIG. 32B) organ weights (n=10-16 mice per group) determined at the end of study. Statistical comparison to DIO-GAN vehicle control determined by ANOVA followed by Tukey correction for multiple comparisons. *p<0.05; **p<0.01; ***p<0.001; ****p<0.0001.

FIG. 33A and FIG. 33B show Body mass composition at baseline. Body mass composition was well balanced across treatment groups at baseline (Week-1). Bars represent mean (SD) fat tissue (FIG. 33A) and lean tissue (FIG. 33B) mass as a percentage of body weight (% BW, n=10-16 mice per group) determined by whole body EchoMRI at Week-1 of the study. Statistical comparison to DIO-GAN vehicle control determined by ANOVA followed by Tukey correction for multiple comparisons. *p<0.05; **p<0.01; ***p<0.001; ****p<0.0001.

FIG. 34A and FIG. 34B show body mass composition at week 11. Compound 2 alone and in combination with Compound 1 significantly reduced fat tissue mass. Bars represent mean (SD) fat tissue (FIG. 34A) and lean tissue (FIG. 34B) mass as a percentage of body weight (% BW, n=10-16 mice per group) determined by whole body EchoMRI at Week 11 of the study. Statistical comparison to DIO-GAN vehicle control determined by ANOVA followed by Tukey correction for multiple comparisons. *p<0.05; **p<0.01; ***p<0.001; ****p<0.0001.

FIG. 35A and FIG. 35B show plasma and liver total cholesterol. Compound 2 and Compound 1 alone and in combination significantly reduced total cholesterol. Bars represent mean (SD) total cholesterol levels measured at the end of study (n=10-16 mice per group) in plasma (FIG. 35A) and liver (FIG. 35B). Statistical comparison to DIO-GAN vehicle control determined by ANOVA followed by Tukey correction for multiple comparisons. *p<0.05; **p<0.01; ***p<0.001; ****p<0.0001.

FIG. 36A and FIG. 36B show plasma and liver triglycerides. Compound 2 alone and in combination with Compound 1 significantly reduced plasma triglycerides. Bars represent mean (SD) triglyceride levels measured at the end of study (n=10-16 mice per group) in plasma (FIG. 36A) and liver (FIG. 36B). Statistical comparison to DIO-GAN vehicle control determined by ANOVA followed by Tukey correction for multiple comparisons. *p<0.05; **p<0.01; ***p<0.001; ****p<0.0001.

FIG. 37A and FIG. 37B shows alanine aminotransferase (ALT) and aspartate aminotransferase (AST) levels. Compound 1 alone significantly reduced Alanine Aminotransferase (ALT) levels. Bars represent mean (SD) ALT (FIG. 37A) and Aspartate Aminotransferase (AST) (FIG. 37B) levels (n=10-16 mice per group) determined at the end of study. Statistical comparison to DIO-GAN vehicle control determined by ANOVA followed by Tukey correction for multiple comparisons. *p<0.05; **p<0.01; ***p<0.001; ****p<0.0001.

FIG. 38 shows alkaline phosphatase levels. Alkaline Phosphatase (ALP) levels were not significantly changed by treatment. Bars represent mean (SD) ALP levels (n=10-16 mice per group) determined at the end of study. Statistical comparison to DIO-GAN vehicle control determined by ANOVA followed by Tukey correction for multiple comparisons. *p<0.05; **p<0.01; ***p<0.001; ****p<0.0001.

FIG. 39A and FIG. 39B show NAFLD Activity Score (NAS) at Baseline and at End of Treatment. The NAFLD Activity Score (NAS) was well balanced at baseline and significantly improved in combination treatment groups. NAS, defined as the composite, unweighted sum of ballooning, steatosis, and lobular inflammation histological scores was determined at baseline (FIG. 39A) and after 12-weeks of treatment (FIG. 39B). Dots represent individual mice in each treatment group (n=14-16). Statistical comparison to DIO-GAN vehicle control determined by ANOVA followed by Tukey correction for multiple comparisons. *p <0.05, **p<0.01, ***p<0.001, ****p<0.0001.

FIG. 40A and FIG. 40B show liver steatosis by histological morphometric analysis. The combination of Compound 2 and Compound 1 resulted in greater reductions in liver steatosis as determined by histological morphometric analysis. Hepatocellular steatosis including the percentage of hepatocytes with lipid droplets and liver lipid content as a percent fractional area (FA) was determined by morphometric analysis of liver histological samples at the end of study. Statistical comparison to DIO-GAN vehicle control determined by ANOVA followed by Tukey correction for multiple comparisons. *p<0.05, **p<0.01, ***p<0.001, ****p<0.0001.

FIG. 41 shows hepatocyte lipid droplet size. Combination treatment significantly reduces hepatocyte lipid droplet size. Lipid droplet size was determined by morphometric analysis of liver histological samples at the end of study. Statistical comparison to DIO-GAN vehicle control determined by ANOVA followed by Tukey correction for multiple comparisons. *p<0.05, **p<0.01, ***p<0.001, ****p<0.0001.

FIG. 42 shows Plasma CK18 M30. Apoptosis biomarker cytokeratin 18 M30 (CK18 M30) levels were not significantly changed by treatment. CK18 M30, an apoptosis biomarker, was measured in plasma samples at the end of study. Statistical comparison to DIO-GAN vehicle control determined by ANOVA followed by Tukey correction for multiple comparisons. *p<0.05, **p<0.01, ***p<0.001, ****p<0.0001.

FIG. 43A and FIG. 43B show liver protein expression of Galectin-3 and smooth muscle actin proteins. Compound 2 treatment reduces expression of Galectin-3 (Gal-3). Expression of Gal-3 (FIG. 43A) and α-smooth muscle actin (α-SMA) (FIG. 43B) was assessed by immunohistochemical (IHC) staining of the livers of treated mice at the end of study. Statistical comparison to DIO-GAN vehicle control determined by ANOVA followed by Tukey correction for multiple comparisons. *p<0.05, **p<0.01, ***p<0.001, ****p<0.0001.

FIG. 44 shows expression of energy and lipid metabolic genes in the liver. Comparison of gene expression values between Compound 1-high and Combo-high treatment groups for select genes involved in energy and lipid metabolism. Liver samples were processed for transcriptomics analysis by RNAseq at termination. Dots represent mean fold-change (n=10 mice per group) expression values relative to DIO-GAN vehicle control. Red and blue coloring indicates the change direction (red=increased expression; blue=decreased expression) between the DIO-GAN vehicle and Lean control groups. Dotted line indicates line of identity. Solid line was generated by linear regression analysis of the fold-change expression values for select genes in the Compound 1-high and Combo-high treatment groups. Select genes involved in energy and lipid metabolism. Squalene epoxidase (Sqle), 7-dehydrocholesterol (Dhcr7), hydroxymethylgluteraryl-CoA synthase (Hmgcs1), and stearoyl-CoA desaturase (Scd1).

FIG. 45A, FIG. 45B, FIG. 45C, and FIG. 45D show expression of select genes involved in energy and lipid metabolism. Liver expression of genes involved in energy and lipid metabolism. Liver samples were processed for transcriptomics analysis by RNAseq at termination. Bars represent mean (SD) expression (FPKM) values for select genes involved in energy and lipid metabolism shown. Squalene epoxidase (Sqle, FIG. 45A), 7-dehydrocholesterol (Dhcr7, FIG. 45B), hydroxymethylgluteraryl-CoA synthase (Hmgcs1, FIG. 45C), and stearoyl-CoA desaturase (Scd1, FIG. 45D). Lean, white (left-most bar); DIO-GAN vehicle control, gray (second from left); Compound 2, blue (third from left); Compound 1-low, light orange (fourth from left); Compound 1-med, orange (fifth from left); Compound 1-high, dark orange (fourth from right); Combo-low, light purple (third from right); Combo-med, purple (second from right); Combo-high, dark purple (right-most bar). Statistical comparison for individual treatment group are shown relative to DIO-GAN vehicle control *p<0.05, **p<0.01, ***p<0.001, ****p<0.0001. Combination treatment groups were additionally compared to Compound 2 (+p<0.05, ++p<0.01, +++p<0.0001, ++++p<0.00001) or their respective Compound 1 single agent treatment groups (i.e., Compound 1-low vs Combo-low); #p<0.05, ##p<0.01, ## #p<0.0001, ## ##p<0.00001.

FIG. 46 shows expression of genes involved in fibrosis and inflammation. Liver expression of genes involved in fibrosis and inflammation. Liver samples were processed for transcriptomics analysis by RNAseq at termination. Bars represent mean (SD) expression (FPKM) values for select genes involved in fibrosis and inflammation. Collagen type I alpha 1 (Col1a1), actin alpha 2 smooth actin (Acta2), Galectin 3 (Lgals3), and melanoma cell adhesion molecule (CD146). Lean, white (left-most bar); DIO-GAN vehicle control, gray (second from left); Compound 2, blue (third from left); Compound 1-low, light orange (fourth from left); Compound 1-med, orange (fifth from left); Compound 1-high, dark orange (fourth from right); Combo-low, light purple (third from right); Combo-med, purple (second from right); Combo-high, dark purple (right-most bar). Statistical comparison for individual treatment group are shown relative to DIO-GAN vehicle control *p<0.05, **p<0.01, ***p<0.001, ****p<0.0001.

### DETAILED DESCRIPTION

### Definitions

As used herein, the following definitions shall apply unless otherwise indicated. Further, if any term or symbol used herein is not defined as set forth below, it shall have its ordinary meaning in the art.

“Comprising” is intended to mean that the compositions and methods include the recited elements, but not exclude others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of, e.g., other ingredients and substantial method steps recited. Embodiments defined by each of these transition terms are within the scope of this invention.

“Combination therapy” or “combination treatment” refers to the use of two or more drugs or agents in treatment, e.g., the use of compound 1 as utilized herein together with another agent useful to treat liver disorders, such as NAFLD, NASH, and symptoms and manifestations of each thereof is a combination therapy. Administration in “combination” refers to the administration of two agents (e.g., compound 1 as utilized herein, and another agent) in any manner in which the pharmacological effects of both manifest in the patient at the same time. Thus, administration in combination does not require that a single pharmaceutical composition, the same dosage form, or even the same route of administration be used for administration of both agents or that the two agents be administered at precisely the same time. Both agents can also be formulated in a single pharmaceutically acceptable composition. A non-limiting example of such a single composition is an oral composition or an oral dosage form. For example, and without limitation, it is contemplated that compound 1 can be administered in combination therapy with another agent in accordance with the present invention.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“Patient” refers to mammals and includes humans and non-human mammals.

Examples of patients include, but are not limited to mice, rats, hamsters, guinea pigs, pigs, rabbits, cats, dogs, goats, sheep, cows, and humans. In some embodiments, patient refers to a human.

“Pharmaceutically acceptable” refers to safe and non-toxic, preferably for in vivo, more preferably, for human administration.

“Pharmaceutically acceptable salt” refers to a salt that is pharmaceutically acceptable. A compound described herein may be administered as a pharmaceutically acceptable salt.

“Salt” refers to an ionic compound formed between an acid and a base. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. Exemplary and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionality, such salts include, without limitation, salts of organic acids, such as carboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisulfate, mono-, di-, and tribasic phosphate, mesylate, tosylate, and the likes.

“Therapeutically effective amount” or dose of a compound or a composition refers to that amount of the compound or the composition that results in reduction or inhibition of symptoms or a prolongation of survival in a patient. The results may require multiple doses of the compound or the composition.

“Treatment” or “treating” refers to an approach for obtaining beneficial or desired results including clinical results. For purposes of this invention, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of the invention contemplate any one or more of these aspects of treatment.

As used herein, “delaying” development of a disease means to defer, hinder, slow, retard, stabilize and/or postpone development of the disease and/or slowing the progression or altering the underlying disease process and/or course once it has developed. This delay can be of varying lengths of time, depending on the history of the disease and/or individual being treated. As is evident to one skilled in the art, a sufficient or significant delay can, in effect, encompass prevention, in that the individual does not develop clinical symptoms associated with the disease. A method that “delays” development of a disease is a method that reduces probability of disease development in a given time frame and/or reduces extent of the disease in a given time frame, when compared to not using the method, including stabilizing one or more symptoms resulting from the disease.

An individual who is “at risk” of developing a disease may or may not have detectable disease, and may or may not have displayed detectable disease prior to the treatment methods described herein. “At risk” denotes that an individual has one or more so-called risk factors, which are measurable parameters that correlate with development of a disease. An individual having one or more of these risk factors has a higher probability of developing the disease than an individual without these risk factor(s). These risk factors include, but are not limited to, age, sex, race, diet, history of previous disease, presence of precursor disease and genetic (i.e., hereditary) considerations. Compounds may, in some embodiments, be administered to a subject (including a human) who is at risk or has a family history of the disease or condition.

The terms “optional” or “optionally” as used throughout the specification means that the subsequently described event or circumstance may but need not occur, and that the description includes instances where the event or circumstance occurs and instances in which it does not. For example, “the nitrogen atom is optionally oxidized to provide for the N-oxide (N→O) moiety” means that the nitrogen atom may but need not be oxidized, and the description includes situations where the nitrogen atom is not oxidized and situations where the nitrogen atom is oxidized.

As used herein, the term “substantially as shown in” when referring, for example, to an XRPD pattern, includes a pattern or graph that is not necessarily identical to those depicted herein, but falls within the limits of experimental errors or deviations when considered by one of ordinary skill in the art.

### Pharmaceutically Acceptable Compositions and Formulations

Pharmaceutically acceptable compositions or simply “pharmaceutical compositions” of Compound 1, a thyroid hormone receptor beta (THR-β) agonist,

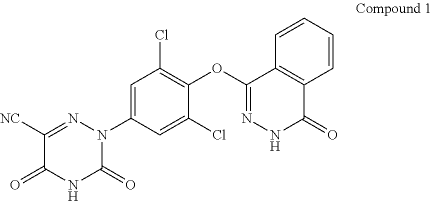 [SMILES: N#Cc1nn(-c2cc(Cl)c(Oc3n[nH]c(=O)c4ccccc34)c(Cl)c2)c(=O)[nH]c1=O]

are embraced by this invention. Thus, the invention includes pharmaceutical compositions comprising Compound 1 or a pharmaceutically acceptable salt thereof, and a pharmaceutically acceptable carrier or excipient. In some embodiments, the pharmaceutically acceptable salt is a base addition salt, such as a salt formed with an inorganic or organic base. In some embodiments, the pharmaceutically acceptable salt is the potassium salt of compound 1. In some embodiments, the pharmaceutically acceptable salt is the sodium salt of compound 1. Pharmaceutical compositions according to the invention may take a form suitable for oral, buccal, parenteral, nasal, topical or rectal administration or a form suitable for administration by inhalation.

Compound 1 as detailed herein may in one aspect be in a purified form and compositions comprising Compound 1 in purified forms are detailed herein. Compositions comprising Compound 1 as detailed herein or a salt thereof are provided, such as compositions of substantially pure compounds. In some embodiments, a composition containing Compound 1 as detailed herein or a salt thereof is in substantially pure form. In one variation, “substantially pure” intends a composition that contains no more than 35% impurity, wherein the impurity denotes a compound other than the compound comprising the majority of the composition or a salt thereof. For example, a composition of a substantially pure compound intends a composition that contains no more than 35% impurity, wherein the impurity denotes a compound other than the compound or a salt thereof. In one variation, a composition of substantially pure compound or a salt thereof is provided wherein the composition contains no more than 25% impurity. In another variation, a composition of substantially pure compound or a salt thereof is provided wherein the composition contains or no more than 20% impurity. In still another variation, a composition of substantially pure compound or a salt thereof is provided wherein the composition contains or no more than 10% impurity. In a further variation, a composition of substantially pure compound or a salt thereof is provided wherein the composition contains or no more than 5% impurity. In another variation, a composition of substantially pure compound or a salt thereof is provided wherein the composition contains or no more than 3% impurity. In still another variation, a composition of substantially pure compound or a salt thereof is provided wherein the composition contains or no more than 1% impurity. In a further variation, a composition of substantially pure compound or a salt thereof is provided wherein the composition contains or no more than 0.5% impurity. In yet other variations, a composition of substantially pure compound means that the composition contains no more than 15% or preferably no more than 10% or more preferably no more than 5% or even more preferably no more than 3% and most preferably no more than 1% impurity.

In one variation, Compound 1 is a synthetic compound prepared for administration to an individual such as a human. In another variation, compositions are provided containing Compound 1 in substantially pure form. In another variation, the invention embraces pharmaceutical compositions comprising Compound 1 and a pharmaceutically acceptable carrier or excipient. In another variation, methods of administering compound 1 are provided. The purified forms, pharmaceutical compositions and methods of administering the compounds are suitable for any compound or form thereof detailed herein.

Compound 1 may be formulated for any available delivery route, including an oral, mucosal (e.g., nasal, sublingual, vaginal, buccal or rectal), parenteral (e.g., intramuscular, subcutaneous or intravenous), topical or transdermal delivery form. Compound 1 may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

Compound 1 can be used in the preparation of a formulation, such as a pharmaceutical formulation, by combining Compound 1 as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising Compound 1 may also contain other substances which have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21^{st} ed. (2005), which is incorporated herein by reference.

Compounds as described herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are microcrystalline cellulose, mannitol, lactose, corn starch or its derivatives, talc, stearate or its salts, etc. Acceptable carriers for gel capsules with soft shell are, for instance, plant oils, wax, fats, semisolid and liquid polyols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Compound 1 has very low aqueous solubility, even when administered in the salt form. The potassium salt of Compound 1 has a solubility of approximately 2.2 μg/mL in a pH 6.0 buffer and a solubility of approximately 8.0 μg/mL in a pH 8.0 buffer. Solubilizers such as poloxamer 188 result in significant degradation of Compound 1. It has been found that particular ionic surfactants such as sodium lauryl sulfate (SLS) are compatible with Compound 1 and can be co-formulated with Compound 1, and pharmaceutically acceptable salts thereof, with minimal or no degradation of the compound. Co-formulation of Compound 1, or pharmaceutically salts thereof, with ionic surfactants (e.g., SLS) results in dramatic enhancement of solubility at all pH levels.

In some embodiments, provided is a formulation comprising Compound 1 or a pharmaceutically acceptable salt thereof and about 1% by weight to about 10% by weight of SLS. In some embodiments, provided is a formulation comprising Compound 1 or a pharmaceutically acceptable salt thereof and about 1% by weight to about 8% by weight of SLS. In some embodiments, provided is a formulation comprising Compound 1 or a pharmaceutically acceptable salt thereof and about 1.5% by weight to about 8% by weight of SLS. In some embodiments, provided is a formulation comprising compound 1 or a pharmaceutically acceptable salt thereof and about 1.5% by weight to about 5% by weight of SLS. In some embodiments, provided is a formulation comprising Compound 1 or a pharmaceutically acceptable salt thereof and about 5% by weight of SLS. In some embodiments, provided is a formulation comprising a potassium salt of Compound 1 and about 1.5% by weight of sodium lauryl sulfate (SLS).

Other potential solubilizers that can be used in combination with Compound 1, or a pharmaceutically acceptable salt thereof, include docusate sodium, polysorbate, a phospholipid, or D-α-tocopheryl polyethylene glycol succinate (Vitamin E TPGS).

In some embodiments, provided is a formulation comprising Compound 1, or a pharmaceutically acceptable salt thereof, and sodium lauryl sulfate. In some embodiments, provided is a formulation comprising Compound 1, or a pharmaceutically acceptable salt thereof, sodium lauryl sulfate and croscarmellose sodium. In some embodiments, provided is a formulation comprising Compound 1, or a pharmaceutically acceptable salt thereof, sodium lauryl sulfate, croscarmellose sodium, colloidal silicaon dioxide, and magnesium stearate. In some embodiments, provided is a formulation comprising Compound 1, or a pharmaceutically acceptable salt thereof, sodium lauryl sulfate, croscarmellose sodium, colloidal silicaon dioxide, magnesium stearate, a Hypromellose (HPMC) capsule, mannitol, and microcrystalline cellulose.

In some embodiments, provided is Compound 1 formulated in a capsule in any dosage form described herein. In some embodiments, provided is a capsule formulation containing about 0.5 mg to about 50 mg of Compound 1, or a pharmaceutically acceptable salt thereof. In some embodiments, provided is a capsule formulation containing about 0.5 mg, about 1 mg, about 2 mg, about 3 mg, about 5 mg, about 6 mg, about 10 mg, about 15 mg, about 25 mg or about 50 mg of Compound 1, or a pharmaceutically acceptable salt thereof.

In some embodiments, provided is Compound 1 formulated in a capsule with one or more of various other components as listed below in Table A. In some embodiments, provided is Compound 1 formulated in a capsule with one or more of various other components in the amounts as described below in Table A.

TABLE A

| Component | Weight/mg |
|---|---|
| Compound 1 | 1.1-54.3 |
| Microcrystalline Cellulose | 59.3-177.9 |
| Mannitol | 29.6-88.8 |
| Sodium Lauryl Sulfate | 5.0-15.0 |
| Croscarmellose Sodium | 3.0-9.0 |
| Colloidal Silicon Dioxide | 1.0-3.0 |
| Magnesium Stearate | 1.0-3.0 |
| HPMC Capsule, Size 0 | 1 capsule |

| | | |
|---|---|---|
| Total | 100 | 300 |

In some embodiments, provided is Compound 1 formulated in a tablet in any dosage form described herein. In some embodiments, provided is a tablet formulation containing about 0.5 mg to about 50 mg of compound 1. It will be understood that in all embodiments, the weight of Compound 1 refers to the active portion of the molecule (free acid). In embodiments where a salt form of Compound 1 is used, the weight of the salt will be adjusted to ensure that the appropriate amount of the active compound is in the composition. In some embodiments, provided is a tablet formulation containing about 0.5 mg, about 1 mg, about 2 mg, about 3 mg, about 5 mg, about 6 mg, about 10 mg, about 15 mg, about 25 mg or about 50 mg Compound 1.

The present disclosure further encompasses kits (e.g., pharmaceutical packages). The kit provided may comprise the pharmaceutical compositions or the compounds described herein and containers (e.g., drug bottles, ampoules, bottles, syringes and/or subpackages or other suitable containers).

### Methods of Use and Uses

Compounds and compositions described herein may in some aspects be used in treatment or prevention of liver disorders. In some embodiments, the method of treating or preventing a liver disorder in a patient in need thereof comprises administering to the patient compound 1, or a pharmaceutically acceptable salt thereof.

Liver disorders include, without limitation, liver inflammation, fibrosis, and steatohepatitis. In some embodiments, the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In certain embodiments, the liver disorder is selected from: liver fibrosis, alcohol induced fibrosis, steatosis, alcoholic steatosis, NAFLD, and NASH. In one embodiment, the liver disorder is NASH. In another embodiment, the liver disorder is liver inflammation. In another embodiment, the liver disorder is liver fibrosis. In another embodiment, the liver disorder is alcohol induced fibrosis. In another embodiment, the liver disorder is steatosis. In another embodiment, the liver disorder is alcoholic steatosis. In another embodiment, the liver disorder is NAFLD. In one embodiment, the treatment methods provided herein impedes or slows the progression of NAFLD to NASH. In one embodiment, the treatment methods provided herein impedes or slows the progression of NASH. NASH can progress, e.g., to one or more of liver cirrhosis, hepatic cancer, etc. In some embodiments, the liver disorder is NASH. In some embodiments, the patient has had a liver biopsy. In some embodiments, the method further comprising obtaining the results of a liver biopsy.

In some embodiments, provided is a method of treating a liver disorder in a patient in need thereof, wherein the liver disorder is selected from the group consisting of liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

Provided herein are methods of treating or preventing a liver disorder in a patient (e.g., a human patient) in need thereof with Compound 1 or a pharmaceutically acceptable salt thereof, comprising administering a therapeutically effective amount of Compound 1 or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

Also provided herein are methods of impeding or slowing the progression of non-alcoholic fatty liver disease (NAFLD) to non-alcoholic steatohepatitis (NASH) in a patient (e.g., a human patient) in need thereof comprising administering compound 1 or a pharmaceutically acceptable salt thereof. In some embodiments, the methods comprise administering a therapeutically effective amount of Compound 1, or a pharmaceutically acceptable salt thereof. Also provided herein are methods of impeding or slowing the progression of NASH in a patient (e.g., a human patient) in need thereof comprising administering compound 1 or a pharmaceutically acceptable salt thereof. In some embodiments, the methods comprises administering a therapeutically effective amount of Compound 1, or a pharmaceutically acceptable salt thereof.

In some embodiments, provided herein is a method of reducing liver damage comprising administering Compound 1 or a pharmaceutically acceptable salt thereof, to an individual in need thereof, wherein fibrosis is reduced. In some embodiments, the level of expression of one or more markers for fibrosis is reduced. In some embodiments, the level of Ccr2, Col1a1, Col1a2, Col1a3, Cxcr3, Den, Hgf, Il1a, Inhbe, Lox, Loxl1, Loxl2, Loxl3, Mmp2, Pdgfb, Plau, Serpine1, Perpinh1, Snai, Tgfb1, Tgfb3, Thbs1, Thbs2, Timp2, and/or Timp3 expression is reduced. In some embodiments the level of collagen is reduced. In some embodiments, the level of collagen fragments is reduced. In some embodiments, the level of expression of the fibrosis marker is reduced at least 2, at least 3, at least 4, or at least 5-fold. In some embodiments, the level of expression of the fibrosis marker is reduced about 2-fold, about 3-fold, about 4-fold, or about 5-fold.

In some embodiments, provided herein a method of reducing liver damage comprising administering Compound 1 or a pharmaceutically acceptable salt thereof, to an individual in need thereof, wherein inflammation is reduced. In some embodiments, one or more markers of inflammation are reduced. In some embodiments, the level of expression of Adgre1, Ccr2, Ccr5, Il1A, and/or Tlr4 is reduced. In some embodiments, the level of expression of the inflammation marker is reduced at least 2-, at least 3-, at least 4-, or at least 5-fold. In some embodiments, the level of expression of the fibrosis marker is reduced about 2-fold, about 3-fold, about 4-fold, or about 5-fold.

In a patient, alkaline phosphatase, gamma-glutamyl transferase (GGT), alanine aminotransferase (ALT) and/or aspartate aminotransferase (AST) levels can be elevated. In some embodiments, provided herein a method of reducing liver damage comprising administering Compound 1, or a pharmaceutically acceptable salt thereof, wherein the GGT, ALT, and/or AST levels are elevated prior to treatment. In some embodiments, the patient's ALT level is about 2-4-fold greater than the upper limit of normal levels. In some embodiments, the patient's AST level is about 2-4-fold greater than the upper limit of normal levels. In some embodiments, the patient's GGT level is about 1.5-3-fold greater than the upper limit of normal levels. In some embodiments, the patient's alkaline phosphatase level is about 1.5-3-fold greater than the upper limit of normal levels. Methods of determining the levels of these molecules are well known. Normal levels of ALT in the blood range from about 7-56 units/liter. Normal levels of AST in the blood range from about 10-40 units/liter. Normal levels of GGT in the blood range from about 9-48 units/liter. Normal levels of alkaline phosphatase in the blood range from about 53-128 units/liter for a 20 to 50-year-old man and about 42-98 units/liter for a 20- to 50-year-old woman.

Thyroid hormone deficiency is more common in NAFLD and NASH patients (Pagadala M R, Zein C O, Dasarathy S, Yerian L M, Lopez R, Mccullough A J. Prevalence of hypothyroidism in nonalcoholic fatty liver disease. Dig Dis Sci. 2012; 57:528-34.). The thyroid gland produces triiodothyronine (T3) and thyroxine (T4), under the control of thyrotropin (thyroid stimulating hormone, [TSH]) from the anterior pituitary in response to thyrotropin-releasing hormone (TRH) from the hypothalamus. Without being bound by any particular theory, THR-β agonism in the liver has been implicated in lowering free T4 without changes in T3 or TSH, which may be attributed to peripheral thyroid hormone modulation (Taub R, Chiang E, Chabot-Blanchet M, Kelly M J, Reeves R A, Guertin M C, et al. Lipid lowering in healthy volunteers treated with multiple doses of MGL-3196, a liver-targeted thyroid hormone receptor-β agonist. Atherosclerosis. 2013; 230:373-80.; Berry M J, Kates A L, Larsen P R. Thyroid hormone regulates type I deiodinase messenger RNA in rat liver. Mol Endocrinol 1990; 4:743-748.). Evidence of impact on the central thyroid axis may represent an undesirable effect. Accordingly, in some embodiments, administration of Compound 1, or a pharmaceutically acceptable salt thereof, does not result in a change in the patient's serum TSH, free T3, or T4 to levels outside of the normal range.

In some embodiments, administration of Compound 1, or a pharmaceutically acceptable salt thereof, does not result in a change in the patient's cardiac biomarkers, including CK, CK-MB and troponin I, to levels outside the normal range.

Sex hormone binding globulin (SHBG) is produced in the liver and binds to and stabilizes sex hormones, including androgens and estrogens, impacting the total and free fractions of circulating hormones (Hammond 2016). THR-β agonism upregulates SHBG expression, making it a useful PD marker. In some embodiments, administration of compound 1, or a pharmaceutically acceptable salt thereof, results in an increase the patient's serum SHBG. In some embodiments, the increase the patient's serum SHBG occurs within 4 days of administration of Compound 1, or a pharmaceutically acceptable salt thereof. In some embodiments, the patient's serum SHBG increases by at least 5% relative to baseline following administration of compound 1, or a pharmaceutically acceptable salt thereof. In some embodiments, the patient's serum SHBG increases by at least 10% relative to baseline following administration of compound 1, or a pharmaceutically acceptable salt thereof. In some embodiments, the patient's serum SHBG increases by at least 25% relative to baseline following administration of compound 1, or a pharmaceutically acceptable salt thereof.

In some embodiments, the patient is a human. Obesity is highly correlated with NAFLD and NASH, but lean people can also be affected by NAFLD and NASH. Accordingly, in some embodiments, the patient is obese. In some embodiments, the patient is not obese. Obesity can be correlated with or cause other diseases as well, such as diabetes mellitus or cardiovascular disorders. Accordingly, in some embodiments, the patient also has diabetes mellitus and/or a cardiovascular disorder. Without being bound by theory, it is believed that comorbidities, such as obesity, diabetes mellitus, and cardiovascular disorders can make NAFLD and NASH more difficult to treat. Conversely, the only currently recognized method for addressing NAFLD and NASH is weight loss, which would likely have little to no effect on a lean patient.

The risk for NAFLD and NASH increases with age, but children can also suffer from NAFLD and NASH, with literature reporting of children as young as 2 years old (Schwimmer, et al., Pediatrics, 2006, 118:1388-1393). In some embodiments, the patient is 2-17 years old, such as 2-10, 2-6, 2-4, 4-15, 4-8, 6-15, 6-10, 8-17, 8-15, 8-12, 10-17, or 13-17 years old. In some embodiments, the patient is 18-64 years old, such as 18-55, 18-40, 18-30, 18-26, 18-21, 21-64, 21-55, 21-40, 21-30, 21-26, 26-64, 26-55, 26-40, 26-30, 30-64, 30-55, 30-40, 40-64, 40-55, or 55-64 years old. In some embodiments, the patient is 65 or more years old, such as 70 or more, 80 or more, or 90 or more.

NAFLD and NASH are common causes of liver transplantation, but patients that already received one liver transplant often develop NAFLD and/or NASH again. Accordingly, in some embodiments, the patient has had a liver transplant.

In some embodiments, treatment in accordance with the methods provided herein results in a reduced NAFLD Activity (NAS) score in a patient. For example, in some embodiments, steatosis, inflammation, and/or ballooning is reduced upon treatment. In some embodiments, the methods of treatment provided herein reduce liver fibrosis. In some embodiments, the methods reduce serum triglycerides. In some embodiments, the methods reduce liver triglycerides.

Dyslipidemia, characterized by elevated low-density lipoprotein (LDL) cholesterol and triglycerides, is a key risk factor for cardiovascular disease (Nelson R H. Hyperlipidemia as a risk factor for cardiovascular disease. Prim Care. 2013 March; 40(1):195-211.) and commonly seen in NASH patients (Loomba R. Nonalcoholic fatty liver disease progression rates to cirrhosis and progression of cirrhosis to decompensation and mortality: a real world analysis of Medicare data. Aliment Pharmacol Ther. 2020; 51:1149-59.). Moreover, dyslipidemia is a potential pathogenic driver of the hepatic inflammation underlying NASH (Walenbergh S. Cholesterol is a significant risk factor for nonalcoholic steatohepatitis. Expert Review of Gastroenterology & Hepatology. 2015; 9:11:1343-46.). Apolipoprotein B (Apo B) is associated with LDL cholesterol in the blood, and may be useful in the assessment of a patient's lipid profile. Accordingly, in some embodiments, administration of compound 1, or a pharmaceutically acceptable salt thereof, decreases the patient's serum Apo B. In some embodiments, administration of Compound 1, or a pharmaceutically acceptable salt thereof, results in a decrease in the patient's serum Apo B within 4 days of administration. In some such embodiments, the decrease in the patient's serum Apo B is at least 5% relative to baseline. In some such embodiments, the decrease in the patient's serum Apo B is at least 10% relative to baseline. In some such embodiments, the decrease in the patient's serum Apo B is at least 15% relative to baseline.

In some embodiments, the patient is at risk of developing an adverse effect prior to the administration in accordance with the methods provided herein. In some embodiments, the adverse effect is an adverse effect which affects the kidney, lung, heart, and/or skin. In some embodiments, the adverse effect is pruritus.

In some embodiments, the patient has had one or more prior therapies. In some embodiments, the liver disorder progressed during the therapy. In some embodiments, the patient suffered from pruritus during at least one of the one or more prior therapies.

Preclinical animal models with Compound 1 established that a steady state plasma area under the curve from time 0 to infinity (AUC_{0-∞}) a approximately 3,320 ng*hg/mL was sufficient exposure to ensure efficacy based on various pharmacodynamics markers, as described in the examples below. Compound 1 exposures of 3,320 ng*hg/mL or greater do not result in undesirable thyroid hormone effects often associated with THR agonism. For instance, in single ascending dose studies described in the examples, steady state plasma AUC_{0-∞} of Compound 1 of up to approximately 50,000 ng*hg/mL did not result in any significant side effects.

In some embodiments, Compound 1, or a pharmaceutically acceptable salt thereof, is administered daily to the individual in need thereof (e.g., a patient with NASH) at a dose to obtain a steady state plasma area under the curve from time 0 to infinity (AUC_{0-∞}) of from about 2,500 ng*h/mL to about 50,000 ng*h/mL. In some embodiments, Compound 1, or a pharmaceutically acceptable salt thereof, is administered daily to the individual in need thereof (e.g., a patient with NASH) at a dose to obtain a steady state plasma area under the curve from time 0 to infinity (AUC_{0-∞}) of from about 3,000 ng*h/mL to about 50,000 ng*h/mL. In some embodiments, Compound 1, or a pharmaceutically acceptable salt thereof, is administered daily to the individual in need thereof (e.g., a patient with NASH) at a dose to obtain a steady state plasma area under the curve from time 0 to infinity (AUC_{0-∞}) of from about 5,000 ng*h/mL to about 50,000 ng*h/mL. In some embodiments, Compound 1, or a pharmaceutically acceptable salt thereof, is administered daily to the individual in need thereof (e.g., a patient with NASH) at a dose to obtain a steady state plasma area under the curve from time 0 to infinity (AUC_{0-∞}) of from about 5,000 ng*h/mL to about 30,000 ng*h/mL. In some embodiments, Compound 1, or a pharmaceutically acceptable salt thereof, is administered daily to the individual in need thereof (e.g., a patient with NASH) at a dose to obtain a steady state plasma area under the curve from time 0 to infinity (AUC_{0-∞}) of from about 5,000 ng*h/mL to about 25,000 ng*h/mL. In some embodiments, Compound 1, or a pharmaceutically acceptable salt thereof, is administered daily to the individual in need thereof (e.g., a patient with NASH) at a dose to obtain a steady state plasma area under the curve from time 0 to infinity (AUC_{0-∞}) of from about 5,000 ng*h/mL to about 20,000 ng*h/mL. In some embodiments, Compound 1, or a pharmaceutically acceptable salt thereof, is administered daily to the individual in need thereof (e.g., a patient with NASH) at a dose to obtain a steady state plasma area under the curve from time 0 to infinity (AUC_{0-∞}) of from about 3,000 ng*h/mL to about 10,000 ng*h/mL. In some embodiments, Compound 1, or a pharmaceutically acceptable salt thereof, is administered daily to the individual in need thereof (e.g., a patient with NASH) at a dose to obtain a steady state plasma area under the curve from time 0 to infinity (AUC_{0-∞}) of from about 5,000 ng*h/mL to about 10,000 ng*h/mL.

Surprisingly, it has been discovered that Compound 1, or a pharmaceutically acceptable salt thereof, can achieve the desired exposure at very low doses. In some embodiments of the disclosure, Compound 1, or a pharmaceutically acceptable salt thereof, can be administered to patients orally once daily at doses as low as 1 mg or less and still sufficiently reduce amine oxidase activity and reduce lymphocyte adhesion and transmigration. For example, Compound 1, or a pharmaceutically acceptable salt thereof, can be administered orally once daily to a patient with a liver disorder (e.g. NASH) at a dose of from about 1 mg to about 60 mg. In some embodiments, Compound 1, or a pharmaceutically acceptable salt thereof, can be administered orally once daily to a patient with a liver disorder (e.g. NASH) at a dose of from about 0.5 mg to about 25 mg. In some embodiments, Compound 1, or a pharmaceutically acceptable salt thereof, can be administered orally once daily to a patient with a liver disorder (e.g. NASH) at a dose of from about 1 mg to about 15 mg. In some embodiments, Compound 1, or a pharmaceutically acceptable salt thereof, can be administered orally once daily to a patient with a liver disorder (e.g. NASH) at a dose of from about 2 mg to about 10 mg. In some embodiments, Compound 1, or a pharmaceutically acceptable salt thereof, can be administered orally once daily to a patient with a liver disorder (e.g. NASH) at a dose of about 1 mg. In some embodiments, Compound 1, or a pharmaceutically acceptable salt thereof, can be administered orally once daily to a patient with a liver disorder (e.g. NASH) at a dose of about 3 mg. In some embodiments, Compound 1, or a pharmaceutically acceptable salt thereof, can be administered orally once daily to a patient with a liver disorder (e.g. NASH) at a dose of about 4 mg. In some embodiments, Compound 1, or a pharmaceutically acceptable salt thereof, can be administered orally once daily to a patient with a liver disorder (e.g. NASH) at a dose of about 5 mg. In some embodiments, Compound 1, or a pharmaceutically acceptable salt thereof, can be administered orally once daily to a patient with a liver disorder (e.g. NASH) at a dose of about 6 mg. In some embodiments, Compound 1, or a pharmaceutically acceptable salt thereof, can be administered orally once daily to a patient with a liver disorder (e.g. NASH) at a dose of about 10 mg. In some embodiments, Compound 1, or a pharmaceutically acceptable salt thereof, can be administered orally once daily to a patient with a liver disorder (e.g. NASH) at a dose of about 15 mg. In some embodiments, Compound 1, or a pharmaceutically acceptable salt thereof, can be administered orally once daily to a patient with a liver disorder (e.g. NASH) at a dose of about 20 mg. It will be understood that in all embodiments, the weight of Compound 1 refers to the active portion of the molecule (free acid). In some embodiments, Compound 1, or a pharmaceutically acceptable salt thereof, can be administered orally once daily to a patient with a liver disorder (e.g. NASH) at a dose of about 30 mg. In some embodiments, Compound 1, or a pharmaceutically acceptable salt thereof, can be administered orally once daily to a patient with a liver disorder (e.g. NASH) at a dose of about 50 mg. In some embodiments, Compound 1, or a pharmaceutically acceptable salt thereof, can be administered orally once daily to a patient with a liver disorder (e.g. NASH) at a dose of about 60 mg. In embodiments where a salt form of Compound is used, the weight of the salt will be adjusted to ensure that the appropriate amount of the active compound is in the composition.

The treatment period generally can be one or more weeks. In some embodiments, the treatment period is at least 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more. In some embodiments, the treatment period is from about a week to about a month, from about a month to about a year, from about a year to about several years. In some embodiments, the treatment period at least any of about 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more. In some embodiments, the treatment period is the remaining lifespan of the patient.

The administration of Compound 1 or a pharmaceutically acceptable salt thereof can be once daily, twice daily or every other day, for a treatment period of one or more weeks. In some embodiments, the administration comprises administering the compound daily for a treatment period of one or more weeks. In some embodiments, the administration comprises administering the compound twice daily for a treatment period of one or more weeks. In some embodiments, the administration comprises administering the compound every other day for a treatment period of one or more weeks.

In some embodiments, the amount of Compound 1, or a pharmaceutically acceptable salt thereof, administered on day 1 of the treatment period is greater than or equal to the amounts administered on all subsequent days of the treatment period. In some embodiments, the amount administered on day 1 of the treatment period is equal to the amounts administered on all subsequent days of the treatment period.

In some embodiments, administration of Compound 1, or a pharmaceutically acceptable salt thereof, decreases steatosis in the individual. Methods of assessing steatosis are known to the skilled artisan and may include histological analysis and assignment of histological score. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing histological markers associated with steatosis.

In some embodiments, administration of Compound 1, or a pharmaceutically acceptable salt thereof, decreases liver inflammation in the individual. Methods of assessing liver inflammation are known to the skilled artisan and may include histological analysis and assignment of histological score of lobular inflammation. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing lobular inflammation or histological markers associated with lobular inflammation.

In some embodiments, administration of Compound 1, or a pharmaceutically acceptable salt thereof, decreases liver fibrosis in the individual. Methods of assessing liver fibrosis are known to the skilled artisan and may include histological analysis. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing fibrosis or histological markers associated with fibrosis.

In some embodiments, administration of Compound 1, or a pharmaceutically acceptable salt thereof, decreases at least one or at least two of liver steatosis, inflammation, and fibrosis in the individual. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing at least one or at least two of steatosis, lobular inflammation, fibrosis, or histological markers of any of the foregoing.

In some embodiments, administration of Compound 1, or a pharmaceutically acceptable salt thereof, decreases serum triglycerides in the individual. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing serum triglycerides.

In some embodiments, administration of Compound 1, or a pharmaceutically acceptable salt thereof, decreases serum total cholesterol in the individual. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing serum cholesterol.

In some embodiments, administration of Compound 1, or a pharmaceutically acceptable salt thereof, decreases serum alanine aminotransferase in the individual. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing serum alanine aminotransferase.

In some embodiments, administration of Compound 1, or a pharmaceutically acceptable salt thereof, decreases at least one or at least two of serum triglycerides, total cholesterol, and alanine aminotransferase in the individual. In some embodiments, administration of compound 1, or a pharmaceutically acceptable salt thereof, decreases serum triglycerides, total cholesterol, and alanine aminotransferase in the individual. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing at least one or at least two of serum triglycerides, total cholesterol, and alanine aminotransferase.

In some embodiments of the foregoing, Compound 1, or a pharmaceutically acceptable salt thereof, is administered to a patient that has not eaten for at least 10 hours prior to dosing. In some embodiments, the compound of compound 1, or a pharmaceutically acceptable salt thereof, is administered to a patient that has consumed a high-fat, high-calorie meal less than 30 minutes prior to dosing.

### Polymorphs

In one aspect, provided herein is a polymorph of Compound 1, or a pharmaceutically acceptable salt thereof. The polymorphs may have properties such as bioavailability and stability under certain conditions that are suitable for medical or pharmaceutical uses.

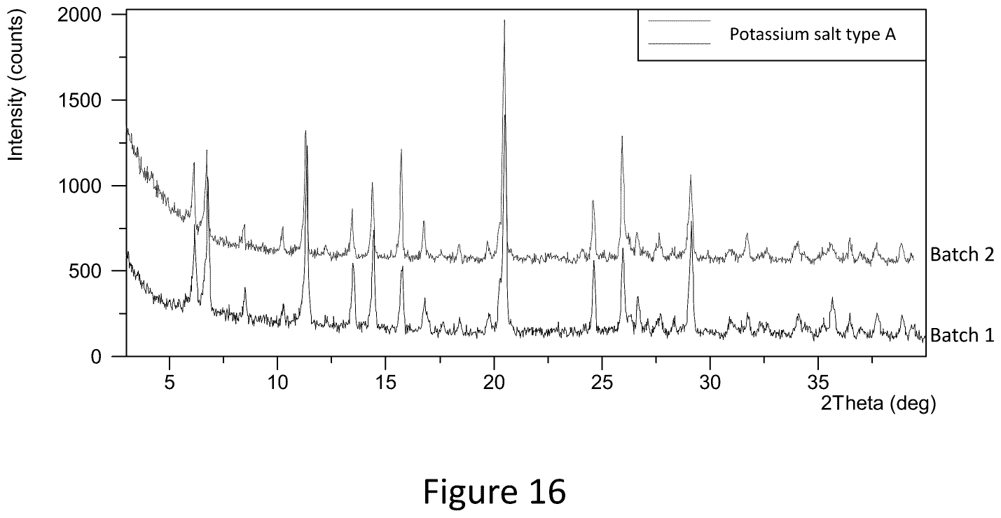

In some embodiments, provided herein is a polymorphic type A of a potassium salt of Compound 1. In some embodiments, type A of a potassium salt of Compound 1 has an XRPD pattern substantially as shown in FIG. 16. Angles 2-theta and relative peak intensities that may be observed for type A of a potassium salt of Compound 1 using XRPD are shown in Table B below.

TABLE B

| Angle [°2θ] | Rel. Int. [%] |
|---|---|
| 6.16 | 37.17 |
| 6.78 | 61.40 |
| 8.49 | 14.10 |
| 11.35 | 79.68 |
| 13.51 | 28.22 |
| 14.44 | 43.58 |
| 15.76 | 27.65 |
| 16.81 | 13.90 |
| 19.77 | 8.02 |
| 20.51 | 100.00 |
| 24.63 | 32.19 |
| 25.97 | 38.26 |
| 26.68 | 15.27 |
| 29.13 | 42.93 |
| 31.00 | 6.24 |
| 31.75 | 10.08 |
| 34.09 | 9.97 |
| 35.67 | 16.95 |
| 36.47 | 8.52 |
| 37.74 | 10.88 |
| 38.90 | 10.20 |

In some embodiments, polymorphic type A of a potassium salt of Compound 1 has an XRPD pattern displaying at least two, at least three, at least four, at least five, at least six, at least seven, at least eight, at least nine, or at least ten of the peaks at angles 2-theta with the greatest intensity in the XRPD pattern as shown in FIG. 16 or as provided in Table B.

In some embodiments, polymorphic type A of a potassium salt of Compound 1 has an XRPD pattern comprising peaks at angles 2-theta of 6.78±0.20, 11.35±0.20, and 20.51±0.20 degrees. In some embodiments, polymorphic type A of a potassium salt of Compound 1 has an XRPD pattern comprising peaks at angles 2-theta of 6.78±0.20, 11.35±0.20, 14.44±0.20, 20.51±0.20, and 29.13±0.20 degrees. In some embodiments, polymorphic type A of a potassium salt of Compound 1 has an XRPD pattern comprising peaks at angles 2-theta of 6.16±0.20, 6.78±0.20, 11.35±0.20, 13.51±0.20, 14.44±0.20, 15.76±0.20, 20.51±0.20, 24.63±0.20, 25.97±0.20, and 29.13±0.20 degrees.

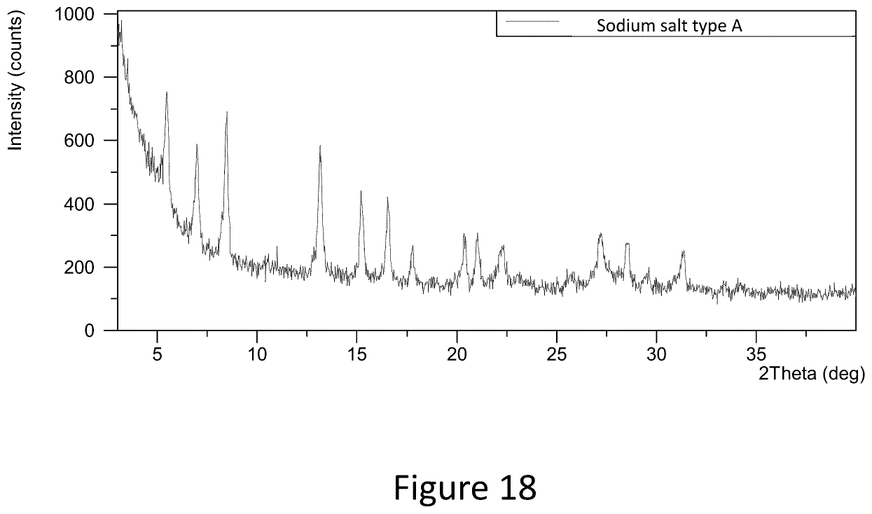

In some embodiments, provided herein is a polymorphic type A of a sodium salt of Compound 1. In some embodiments, type A of a sodium salt of Compound 1 has an XRPD pattern substantially as shown in FIG. 18. Angles 2-theta and relative peak intensities that may be observed for type A of a sodium salt of Compound 1 using XRPD are shown in Table C below.

TABLE C

| Pos. [°2θ] | Rel. Int. [%] |
|---|---|
| 5.51 | 100.00 |
| 6.99 | 62.36 |
| 8.47 | 98.20 |
| 11.04 | 7.64 |
| 13.12 | 55.02 |
| 15.24 | 60.80 |
| 16.57 | 72.37 |
| 17.78 | 24.26 |
| 20.42 | 34.10 |
| 21.02 | 27.00 |
| 22.19 | 24.02 |
| 23.07 | 7.67 |
| 25.69 | 7.77 |
| 27.21 | 17.97 |
| 27.99 | 4.80 |
| 28.55 | 37.46 |
| 29.58 | 6.95 |
| 31.33 | 31.60 |
| 32.49 | 3.54 |
| 33.51 | 6.13 |
| 34.16 | 6.34 |

In some embodiments, polymorphic type A of a sodium salt of Compound 1 has an XRPD pattern displaying at least two, at least three, at least four, at least five, at least six, at least seven, at least eight, at least nine, or at least ten of the peaks at angles 2-theta with the greatest intensity in the XRPD pattern as shown in FIG. 18 or as provided in Table C.

In some embodiments, polymorphic type A of a sodium salt of Compound 1 has an XRPD pattern comprising peaks at angles 2-theta of 5.51±0.20, 8.47±0.20, and 16.57±0.20 degrees. In some embodiments, polymorphic type A of a sodium salt of Compound 1 has an XRPD pattern comprising peaks at angles 2-theta of 5.51±0.20, 6.99±0.20, 8.47±0.20, 15.24±0.20, and 16.57±0.20 degrees. In some embodiments, polymorphic type A of a sodium salt of Compound 1 has an XRPD pattern comprising peaks at angles 2-theta of 5.51±0.20, 6.99±0.20, 8.47±0.20, 13.12±0.20, 15.24±0.20, 16.57±0.20, 20.42±0.20, 21.02±0.20, 28.55±0.20, and 31.33±0.20 degrees.

It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, XRPD peak assignments listed herein, including for polymorphic type A of a potassium salt of Compound 1 or for polymorphic type A of a sodium salt of Compound 1, can vary by ±0.6 degrees, ±0.4 degrees, ±0.2 degrees, or ±0.1 degrees 2-theta. In some embodiments, peak assignments listed herein can vary by ±0.6 degrees 2-theta. In some embodiments, peak assignments listed herein can vary by ±0.4 degrees 2-theta. In some embodiments, peak assignments listed herein can vary by ±0.2 degrees 2-theta. In some embodiments, peak assignments listed herein can vary by ±0.1 degrees 2-theta.

### Combinations

The present disclosure further provides combinations of Compound 1, or a pharmaceutically acceptable salt thereof, with other therapeutic agents that are used to treat liver diseases. In particular, the present disclosure provides for combinations of Compound 1, or a pharmaceutically acceptable salt thereof, and other therapeutic agents used in the treatment of NASH. Owing to its low clinical dose, as disclosed herein, Compound 1, or a pharmaceutically acceptable salt thereof, is an attractive candidate for use in fixed-dose combinations for the treatment of NASH.

In some embodiments, the Compound 1, or a pharmaceutically acceptable salt thereof, is administered in combination with a Farnesoid X Receptor (FXR) agonist. In some embodiments, the FXR agonist is obeticholic acid. In some embodiments, the FXR agonist is cilofexor. In some embodiments, the FXR agonist is tropifexor. In some embodiments, the FXR agonist is EYP001 (Vonafexor, proposed INN). In some embodiments, the FXR agonist is MET409 (Metacrine). In some embodiments, the FXR agonist is EDP-305 (by Enanta). In some embodiments, the FXR agonist is

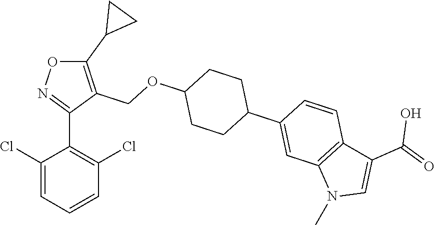 [SMILES: Cn1cc(C(=O)O)c2ccc(C3CCC(OCc4c(-c5c(Cl)cccc5Cl)noc4C4CC4)CC3)cc21]

(or “Compound 2”), or a pharmaceutically acceptable salt thereof.

In some embodiments, the Compound 1, or a pharmaceutically acceptable salt thereof, is administered in combination with a peroxisome proliferator-activated receptor (PPAR) agonist. In some embodiments, the PPAR agonist is pioglitazone. In some embodiments, the PPAR agonist is rosiglitazone. In some embodiments, the PPAR agonist is elalafibranor. In some embodiments, the PPAR agonist is saroglitazar. In some embodiments, the PPAR agonist is lanifibranor. In some embodiments, the PPAR agonist is elafibranor. In some embodiments, the PPAR agonist is seladelphar.

In some embodiments, the Compound 1, or a pharmaceutically acceptable salt thereof, is administered in combination with a pan-caspase inhibitor. In some embodiments, the pan-caspase inhibitor is emricasan.

In some embodiments, the Compound 1, or a pharmaceutically acceptable salt thereof, is administered in combination with a galectin-3 inhibitor. In some embodiments, the galectin-3 inhibitor is belapectin.

In some embodiments, the Compound 1, or a pharmaceutically acceptable salt thereof, is administered in combination with a stearoyl Co-A desaturase 1 inhibitor. In some embodiments, the stearoyl Co-A desaturase 1 inhibitor is armachol.

In some embodiments, the Compound 1, or a pharmaceutically acceptable salt thereof, is administered in combination with a chemokine receptor type 2 and 5 (CCR2/CCR5 chemokine) antagonist. In some embodiments, the CCR2/CCR5 chemokine agonist is cenicriviroc.

In some embodiments, the Compound 1, or a pharmaceutically acceptable salt thereof, is administered in combination with an antioxidant. In some embodiments, the antioxidant is Vitamin E.

In some embodiments, compound 1 is co-administered with a cholesterol lowering drug. In some embodiments, the cholesterol lowering drug is statin. In some such embodiments, In some embodiments, the statin is atorvastatin, simvastatin or rosuvastatin.

### Articles of Manufacture and Kits

The present disclosure further provides articles of manufacture comprising a compound described herein, or a salt thereof, a composition described herein, or one or more unit dosages described herein in suitable packaging. In certain embodiments, the article of manufacture is for use in any of the methods described herein. Suitable packaging (e.g., containers) is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises compound 1, or a pharmaceutically acceptable salt thereof, or a composition comprising compound 1, or a pharmaceutically acceptable salt thereof. The kits may be used for any one or more of the uses described herein, and, accordingly, may contain instructions for the treatment as described herein.

Kits generally comprise suitable packaging. The kits may comprise one or more containers comprising compound 1 or a pharmaceutically acceptable salt thereof.

The kits may be in unit dosage forms, bulk packages (e.g., multi-dose packages) or sub-unit doses. For example, kits may be provided that contain sufficient dosages of the compound of Formula (I), or a pharmaceutically acceptable salt thereof, and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compound and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### EXAMPLES

### Example 1: Single Ascending Dose Trial of Compound 1 in Healthy Human Subjects

### Methods

A single-ascending dose clinical trial of Compound 1 (potassium salt) was performed. Four groups of 8 healthy participants were randomized to receive Compound 1 (3 mg, 10 mg, 30 mg, or 60 mg capsule) or matching placebo in a 3:1 ratio (n=6 active and n=2 placebo) and were administered during the fasted state on Day 1 of the study. Plasma levels of Compound 1 and PD biomarkers were determined at pre-dose and various time points post-dose.

Adverse event (AE) monitoring, routine clinical laboratory testing (including thyroid axis testing [free and total thyroid hormone triiodothyronine (T3), free and total thyroid hormone thyroxine (T4), thyroid stimulating hormone (TSH)] cardiac biomarkers [CK-MB, troponin I], and liver biochemistry), intensive vital signs, cardiac telemetry, and electrocardiograms were assessed throughout the study. Compound 1 plasma and urine concentrations were determined using validated liquid chromatography-tandem mass spectrometry assay

Plasma samples for Compound 1 concentration and PK sampling were collected at pre-dose and at 0.5, 1, 2, 3, 4, 6, 8, 12, 24, 48, and 72 hours after administration of a single dose of study medication (placebo or compound). Urine samples for Compound 1 concentration and PK sampling were collected pre-dose and at the following timepoints: 0-6 hours, 6-12 hours, 12-24 hours, and 24-48 hours. PK parameters were estimated via noncompartmental methods using Phoenix WinNonlink (Certara, LP, Princeton, NJ). Concentrations of serum pharmacodynamic (PD) biomarkers apolipoprotein B (Apo B) and sex hormone binding globulin (SHBG) were measured using an immunoassay and serum lipids were determined using spectrophotometry.

PD sampling was performed pre-dose, and 48 hours and 72 hours post-dose. Percent change from baseline for PD markers were calculated using an ANCOVA model with percent change from baseline as dependent variable, treatment group as fixed effect, and baseline as covariate. Analyses used observed data only without imputation for missing data.

### Results

### Treatment-Emergent Adverse Events Monitoring

All adverse events were mild or moderate in severity and largely unrelated to the study drug. No cardiac related AEs (e.g., tachycardia, arrythmias) were reported, and no remarkable changes in vital signs or ECG parameters were seen (see Table 1).

TABLE 1
Safety Monitoring at Various Doses of Compound 1

| | | 3 mg | 10 mg | 30 mg | 60 mg |
|---|---|---|---|---|---|
| | Placebo | Cmpd 2 | Cmpd 2 | Cmpd 2 | Compd 2 |
| | (n = 8) | (n = 6) | (n = 6) | (n = 6) | (n = 6) |
| Any AE, all CTCAE grades | 1 (12.5%) | 2 (33.3%) | 0 | 2 (33.3%) | 0 |
| CTCAE Grade 1 | 0 | 0 | 0 | 1 (16.7%) | 0 |
| CTCAE Grade 2 | 0 | 0 | 0 | 1 (16.7%) | 0 |
| CTCAE Grade 3 or higher | 0 | 0 | 0 | 0 | 0 |
| Serious AEs | 0 | 0 | 0 | 0 | 0 |

AEs by relationship to drug

| | | | | | |
|---|---|---|---|---|---|
| Not related | 1 (12.5%) | 2 (33.3%) | 0 | 1 (16.7%) | 0 |
| Unlikely related | 0 | 0 | 0 | 0 | 0 |
| Possibly related | 0 | 0 | 0 | 1 (16.7%)* | 0 |
| Related | 0 | 0 | 0 | 0 | 0 |

*One subject reported headache on Day 1 and Day 3 which resolved spontaneously, and pleuritic pain (left axilla) on Day 2 which lasted a few hours and resolved after one dose (1000 mg) of acetaminophen.

### Safety and Thyroid Axis Monitoring

Heart rates remained stable and within the normal range for 24 hours after dosing with Compound 1, following a similar pattern as seen in the placebo group. TSH, free T3, and free T4 levels remained within the normal range. No subjects experienced increases in ALT more than 2-times greater than the upper limit of normal (ULN); no subjects exhibited bilirubin levels above the normal range. In addition, no notable changes in cardiac biomarkers (troponin I, CK-MB) or other clinical safety tests were observed.

### Pharmacokinetics

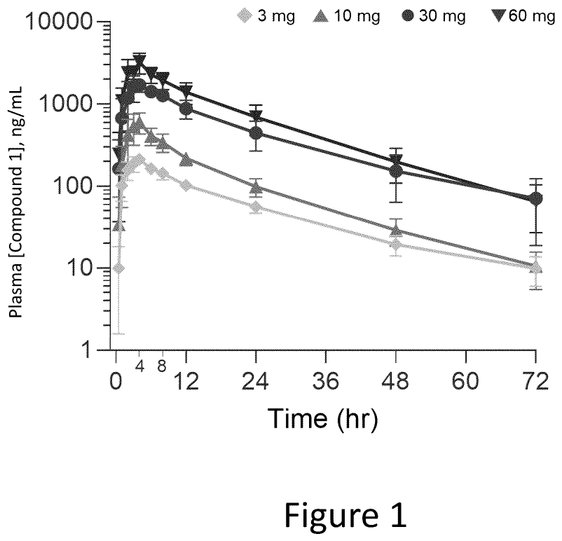

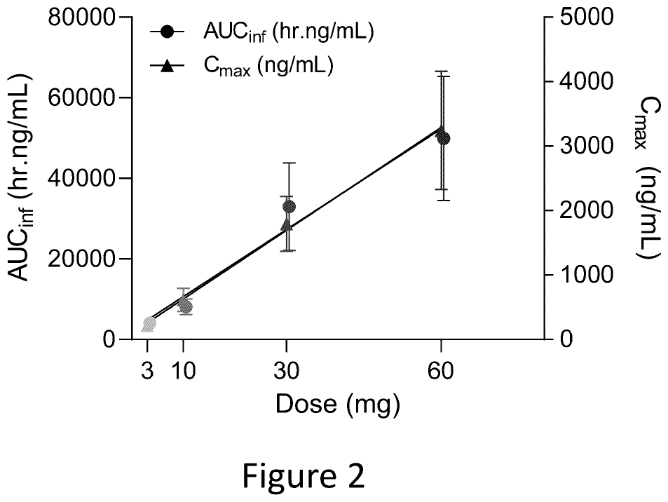

Compound 1 was absorbed with low variability (% CV≤33%) under fasted conditions. Exposures (AUC, Cₘₐₓ) were approximately dose-proportional. Median half-life of Compound 1 ranged from 13.8 to 17.3 h, supporting once daily dosing. Minimal renal excretion was determined at all doses. See Table 2, FIG. 1, FIG. 2.

TABLE 2
Pharmacokinetic Results

| Dose of | AUC_{inf} | Cₘₐₓ | Tₘₐₓ | t_{1/2} | fₑ |
|---|---|---|---|---|---|
| Compound 1 | (hr*ng/mL) | (ng/mL) | (h) | (h) | (% dose, urine) |
| 3 mg (n = 6) | 4060 (16.6) | 217 (13.9) | 4.00 (3.00-4.00) | 17.3 (13.8-20.1) | 2.95 (62) |
| 10 mg (n = 6) | 8110 (23.9) | 614 (29.0) | 4.00 (3.00-4.03) | 14.4 (11.5-16.2) | 2.05 (74) |
| 30 mg (n = 6) | 33000 (33.0) | 1800 (23.7) | 4.00 (3.00-6.00) | 15.7 (11.3-23.1) | 1.78 (35) |
| 60 mg (n = 6) | 50000 (31.4) | 3300 (33.2) | 4.00 (3.00-4.00) | 13.8 (10.5-16.9) | 1.53 (53) |

Parameters are presented to 3 significant figures as mean (% CV), except Tₘₐₓ and t_{1/2} which are presented as median (min-max range). fₑ is fraction of dose excreted in urine as unmodified Compound 1.

### Pharmacodynamics

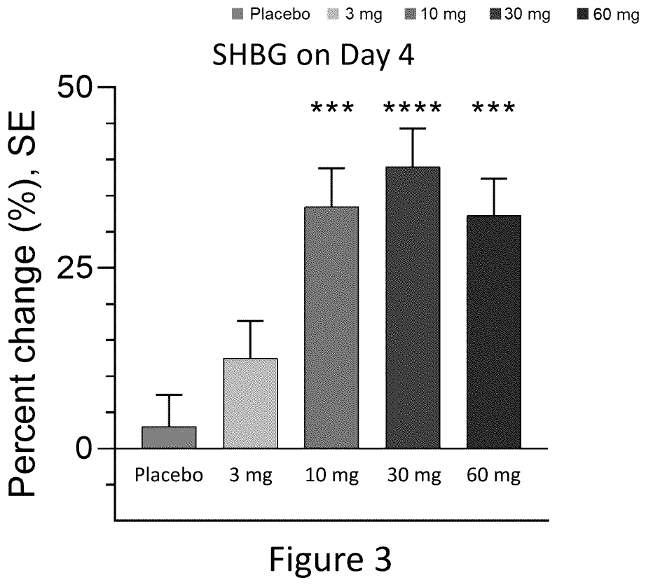

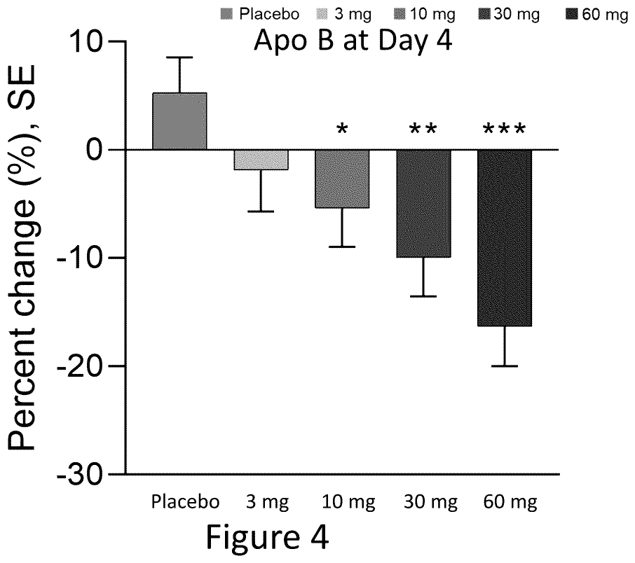

Mean percent change in sex hormone binding globulin (SHBG) and apolipoprotein B (Apo B) at Day 3 after a single dose of Compound 1 on Day 1 of the study are shown in FIG. 3 and FIG. 4, respectively. Mean percent change refer to least squares mean (LSM) from ANCOVA model and standard error (SE). P-value vs. placebo: *<0.05; **<0.01; ***<0.001; ****<0.0001. Significant increases in SHBG were observed following single doses of ≥10 mg Compound 1 relative to placebo. Dose-dependent decreases in LDL-c, total cholesterol, and Apo-B were observed on Day 3 following single dose administration of Compound 1 with similar results on Day 4. No significant changes in triglyceride levels were observed after a single dose of Compound 1 (see Table 3).

TABLE 3
PD marker changes, Day 3

| | | | | |
|---|---|---|---|---|
| Placebo (PBO) | 3 mg | 10 mg | 30 mg | 60 mg |
| (n = 8) | (n = 6) | (n = 6) | (n = 6) | (n = 6) |

SHBG

| | | | | | | | | | | |
|---|---|---|---|---|---|---|---|---|---|---|
| Baseline | 35.0 | (17.97) | 46.7 | (26.30) | 55.0 | (20.63) | 26.0 | (11.19) | 39.3 | (31.97) |
| (nmol/L) | | | | | | | | | | |
| Change | −0.77 | (2.17) | 0.20 | (2.51) | 15.8 | (2.61) | 6.0 | (2.6) | 5.05 | (2.49) |
| (nmol/L) | | | | | | | | | | |
| LSM % change | −3.8 | (3.91) | 6.3 | (4.52) | 31.3 | (4.69) | 16.5 | (4.67) | 13.8 | (4.48) |

| | | | | | |
|---|---|---|---|---|---|
| P-value vs. | — | 0.11 | <0.0001 | 0.0023 | 0.0064 |
| PBO | | | | | |

LDL-c

| | | | | | | | | | | |
|---|---|---|---|---|---|---|---|---|---|---|
| Baseline | 106.1 | (36.06) | 137.5 | (17.4) | 133.7 | (18.2) | 123.3 | (23.55) | 125.2 | (45.96) |
| (nmol/L) | | | | | | | | | | |

| | | | | | | | | | |
|---|---|---|---|---|---|---|---|---|---|
| Change | 5.8 | (3.44) | −3.98 | (3.39) | 0.68 | (3.34) | −15.7 | (3.32) | N/A |
| (nmol/L) | | | | | | | | | |
| LSM % change | 5.6 | (2.85) | −3.1 | (2.80) | 0.70 | (2.76) | −12.5 | (2.75) | N/A |

| | | | | | |
|---|---|---|---|---|---|
| P-value vs. | — | 0.048 | 0.24 | 0.0002 | N/A |
| PBO | | | | | |

HDL-c

| | | | | | | | | | | |
|---|---|---|---|---|---|---|---|---|---|---|
| Baseline | 45.4 | (11.40) | 51.0 | (22.53) | 63.7 | (23.55) | 49.5 | (7.82) | 48.2 | (13.93) |
| (nmol/L) | | | | | | | | | | |

| | | | | | | | | | |
|---|---|---|---|---|---|---|---|---|---|
| Change | 0.29 | (0.93) | 0.87 | (0.91) | −1.1 | (0.96) | −6.1 | (0.91) | N/A |
| (nmol/L) | | | | | | | | | |
| LSM % change | 1.7 | (1.77) | 2.4 | (1.73) | −1.0 | (1.83) | −12.1 | (1.74) | N/A |

| | | | | | |
|---|---|---|---|---|---|
| P-value vs. | — | 0.78 | 0.3027 | <0.0001 | N/A |
| PBO | | | | | |

TC

| | | | | | | | | | | |
|---|---|---|---|---|---|---|---|---|---|---|
| Baseline | 171.7 | (45.05) | 216.5 | (29.87) | 217.7 | (38.10) | 196.2 | (31.2) | 191.2 | (51.68) |
| (nmol/L) | | | | | | | | | | |

| | | | | | | | | | |
|---|---|---|---|---|---|---|---|---|---|
| Change | 2.3 | (4.73) | −1.85 | (4.59) | −1.93 | (4.61) | −24.14 | (4.52) | N/A |
| (nmol/L) | | | | | | | | | |
| LSM % change | 1.6 | (2.15) | −0.88 | (2.09) | −0.84 | (2.09) | −11.84 | (2.06) | N/A |

| | | | | | |
|---|---|---|---|---|---|
| P-value vs. | — | 0.44 | 0.45 | 0.0002 | N/A |
| PBO | | | | | |

Apo B

| | | | | | | | | | | |
|---|---|---|---|---|---|---|---|---|---|---|
| Baseline | 86.7 | (23.18) | 113.5 | (20.06) | 105.8 | (9.26) | 101.2 | (15.89) | 92.3 | (24.9) |
| (nmol/L) | | | | | | | | | | |
| Change | 3.8 | (2.72) | −1.5 | (3.14) | −5.6 | (3.0) | −11.5 | (2.97) | −13.9 | (3.0) |
| (nmol/L) | | | | | | | | | | |
| LSM % change | 5.5 | (2.63) | −2.7 | (3.04) | −6.0 | (2.91) | −11.0 | (2.9) | −15.75 | (2.91) |

| | | | | | |
|---|---|---|---|---|---|
| P-value vs. | — | 0.064 | 0.0081 | 0.0003 | <0.0001 |
| PBO | | | | | |

### Conclusions

Single ascending doses of Compound 1 up to 60 mg were overall safe and well-tolerated and exhibited linear and dose-proportional plasma exposures with low variability. The half-life was >13 hours at all single dose levels, supportive of once daily oral dosing. Renal excretion of unchanged Compound 1 was minimal, indicating renal elimination is a minor pathway.

Significant dose-dependent effects on SHBG, Apo B, and LDL-c were observed following a single dose of Compound 1, indicating the potential for efficacy. Relative to the efficacious dose of Compound 1 in preclinical models (threshold AUC of 3,320 ng*h/mL achieved significant histological improvement in a mouse model of NASH), sufficient exposure levels were achieved in human subjects across all Compound 1 doses.

The safety, PK, and PD results support continued development of Compound 1 and indicate that it is well-suited for co-formulation with other oral small molecule NASH agents as an oral, once-daily fixed dose combination.

### Example 2: Multiple Ascending Dose Trial of Compound 1 in Healthy Human Subjects

A multiple-ascending dose clinical trial of Compound 1 was performed. Four groups of 8 healthy participants were randomized to receive Compound 1 (1 mg, 3 mg, 6 mg, or 10 mg capsule) or matching placebo in a 3:1 ratio (n=6 active and n=2 placebo) and were administered once daily during the fasted state for 14 days. Plasma levels of Compound 1 and PD biomarkers were determined at pre-dose and various time points post-dose.

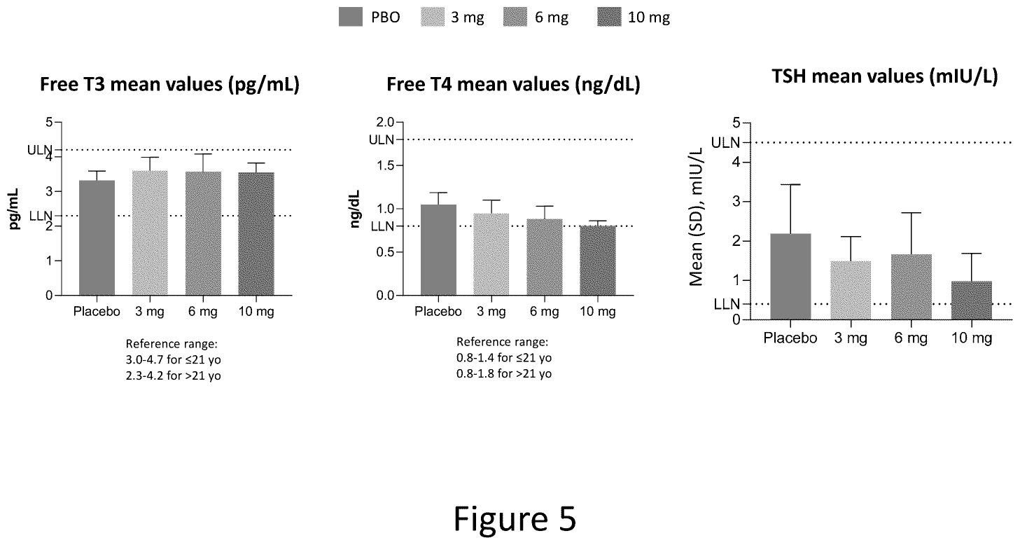

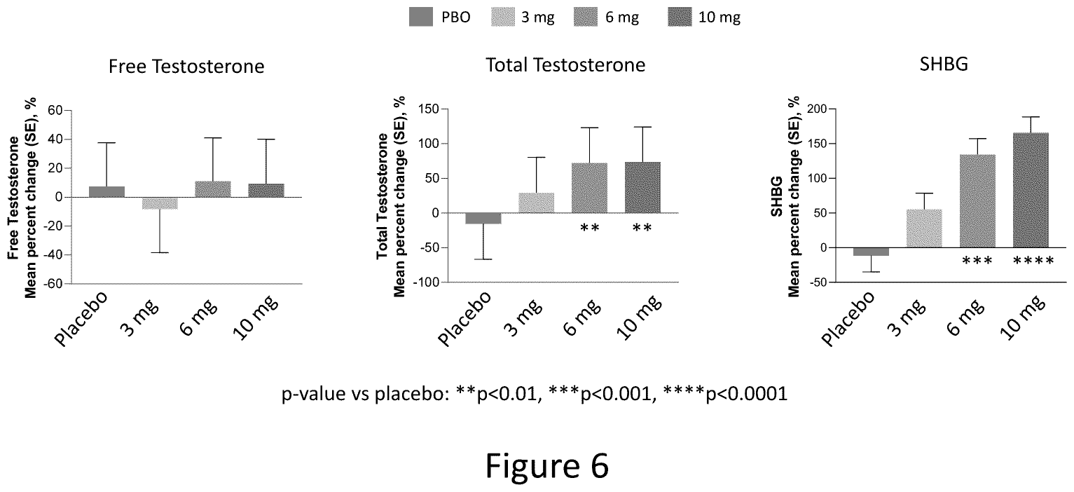

Adverse event (AE) monitoring (Table 4), routine clinical laboratory testing (including thyroid axis testing [free and total thyroid hormone triiodothyronine (T3), free and total thyroid hormone thyroxine (T4), thyroid stimulating hormone (TSH)], cardiac biomarkers [CK-MB, troponin I], and liver biochemistry) (FIG. 5, FIG. 6), intensive vital signs, cardiac telemetry, and electrocardiograms were assessed throughout the study. Compound 1 concentrations were determined using validated liquid chromatography-tandem mass spectrometry assay

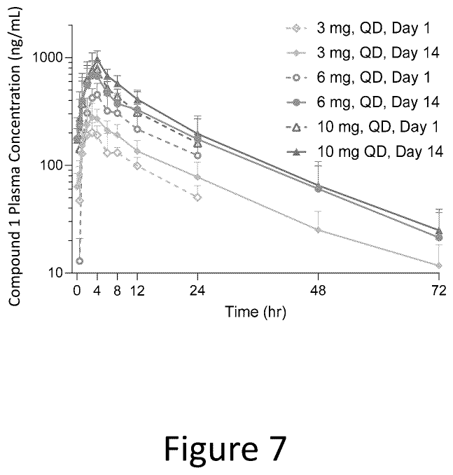

Plasma samples for Compound 1 concentration and PK sampling were collected at pre-dose and at 0.5, 1, 2, 3, 4, 6, 8, 12, and 24 hours after the first dose, at pre-dose on Days 3, 4, 5, 8, 11, and 13 of dosing, and at pre-dose and at 0.5, 1, 2, 3, 4, 6, 8, 12, 24, 48, and 72 hours after 14 days of once daily administration of study medication (placebo or compound). (See Table 5, FIG. 7.) PK parameters were estimated via noncompartmental methods using Phoenix® WinNonlin® (Certara, LP, Princeton, NJ). Concentrations of serum pharmacodynamic (PD) biomarkers apolipoprotein B (Apo B) and sex hormone binding globulin (SHBG) were measured using an immunoassay and serum lipids were determined using spectrophotometry.

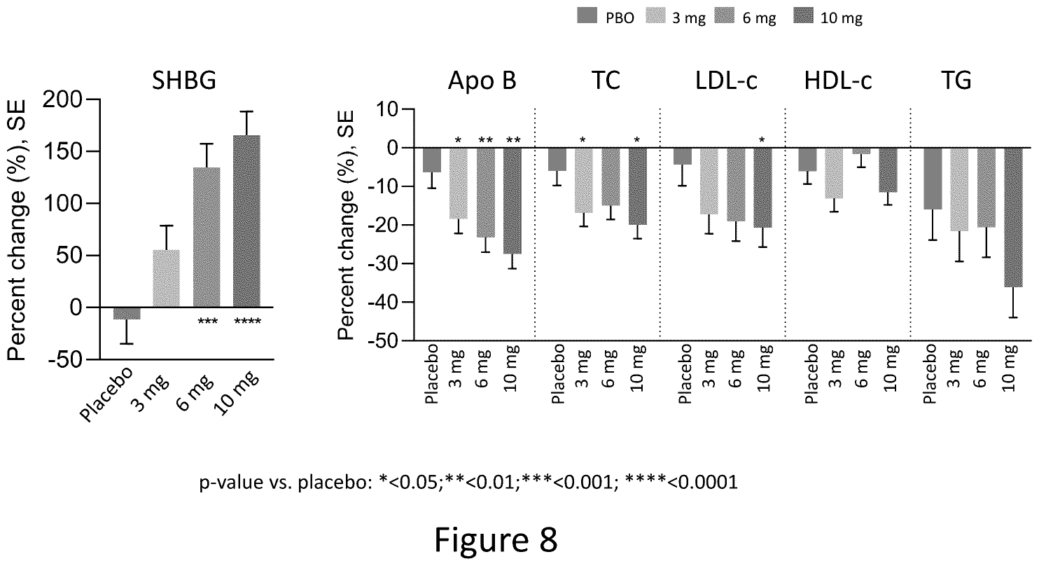

PD sampling was also performed. Percent change from baseline for PD markers were calculated using an ANCOVA model with percent change from baseline as dependent variable, treatment group as fixed effect, and baseline as covariate. Analyses used observed data only without imputation for missing data. PD data on day 15 of the study are shown in FIG. 8.

### Results and Conclusions

### Safety

TABLE 4
Safety and adverse effects

| Subject Incidence | Placebo (N = 6) | 3 mg (N = 6) | 6 mg (N = 6) | 10 mg (N = 6) |
|---|---|---|---|---|
| AEs by category | n (%) | n (%) | n (%) | n (%) |
| Any AE, all CTCAE grades | 0 | 1 (16.7%) | 1 (16.7%) | 2 (33.3%) |
| Grade 1 | 0 | 1 (16.7%) | 1 (16.7%) | 2 (33.3%) |
| Grade 2 or higher | 0 | 0 | 0 | 0 |
| Serious AEs | 0 | 0 | 0 | 0 |

AEs by relationship to drug

| | | | | |
|---|---|---|---|---|
| Not related | 0 | 1 (16.7%) | 0 | 2 (33.3%) |
| Unlikely related | 0 | 0 | 0 | 0 |
| Possibly related | 0 | 0 | 1 (16.7%) | 0 |
| Related | 0 | 0 | 0 | 0 |

*Dizziness was reported in one subject;
CTCAE = common terminology criteria for adverse events, AE = adverse event; Treatment-emergent adverse events (TEAE) are those that occur on or after the time of first dose of study drug; Severity of adverse events was graded according to CTCAE version 5.

14-day daily dosing of placebo or Compound 1 up to 10 mg was overall well-tolerated. No study stopping criteria or dose escalation stopping criteria were met. All AEs were Grade 1 and most considered not related to the study drug. All subjects randomized to Compound 1 completed the study with no study drug discontinuation. Heart rate and blood pressure remained stable throughout the study. Free T4 declined in dose-dependent fashion without clear change in TSH or free T3; T4 changes were asymptomatic and not considered clinically significant, suggesting peripheral thyroid hormone modulation leading to lower free T4 without changes in T3 or TSH.

Mean ALT values were similar across the groups, and not significantly different from placebo; no treated subject had ALT increase to ≥2×ULN. Dose-dependent GGT increases were noted, with values remaining below ULN for treated patients. Dose-dependent total testosterone increases were observed, but no significant change in free levels was identified.

No remarkable changes in ECGs, cardiac biomarkers, or other clinical lab tests were identified.

### Pharmacokinetics and Pharmacodynamics

TABLE 5
Pharmacokinetics

| | | | 3 mg, QD | | 6 mg, QD | | 10 mg, QD | |
|---|---|---|---|---|---|---|---|---|
| Day 14 PK Parameter | 1 mg, QD | | N = 6 | | N = 6 | | N = 6 | |
| AUCₜₐᵤ (hr · ng/mL) | 1090 | (15.7) | 3600 | (26.9) | 8310 | (44.4) | 10600 | (21.1) |
| (mean, % CV) | | | | | | | | |
| Cₘₐₓ (ng/mL) | 80.3 | (20.0) | 291 | (30.9) | 699 | (35.1) | 996 | (18.0) |
| (mean, % CV) | | | | | | | | |
| Cₜₐᵤ (ng/mL) | 25.7 | (28.6) | 77.9 | (37.6) | 175 | (64.7) | 196 | (38.0) |
| (mean, % CV) | | | | | | | | |
| t_{1/2} (hr) | 19.5 | (17.0-23.8) | 16.0 | (14.1-19.6) | 15.8 | (12.7-17.6) | 15.4 | (13.1-18.5) |
| (median, min-max) | | | | | | | | |

Pharmacokinetic data indicated good oral bioavailability with low PK variability. Multiple doses of Compound 1 led to significant and dose-dependent increases in SHBG, even at a low dose of 3 mg QD. Reductions in total cholesterol, LDL-c, Apo B, and triglycerides were observed in all dose levels of Compound 1, with significant reductions observed at day 15 in the 10 mg dose cohort. HDL-c did not significantly change in by day 15. These results support a low once daily dose of Compound 1 is efficacious.

### Example 3: Drug-Drug Interaction Studies with Compound 1 in Healthy Humans

In vitro studies indicated Compound 1 has potential for first pass inhibition of organic anion transporting polypeptides (OATP) 1B1/1B3 and intestinal inhibition of breast cancer resistance protein (BCRP) transporters (OATP1B1 IC50=2.01 micromolar; OATP1B3 IC50-0.71 micromolar; BCRP IC50=9.37 micromolar). The effect of Compound 1 on the pharmacokinetics of coadministered rosuvastatin (ROS), an antihyperlipidemia drug and a substrate of OATP and BCRP, in healthy participants, is determined by administering Compound 1 and ROS as described in Table 6, in combination with PK sampling.

TABLE 6
Co-administration of Compound 1 with Rosuvastatin (ROS)

| | Study Day | | | | | |
|---|---|---|---|---|---|---|
| | 1 | 2 | 3-8 | 9 | 10-11 | 12 |
| Cohort 1 | Single- | No | Up to 90 | Single-dose of | No | Clinic |
| (n = 10) | dose of 10 | Treatment | mg | up to 90 mg | Treatment | Discharge |
| | mg ROS | | Compound 1, | Compound 1 + | | |
| | PO | | QD | Single-dose of | | |
| | | | PO | 10 mg ROS | | |
| | | | | PO | | |

PO = by mouth;
QD = once daily

In addition, Compound 2 is an inhibitor of intestinally expressed transporters P-glycoprotein (P-gp) and BCRP (P-gp IC50=3.92 micromolar; BCRP IC50=4.39 micromolar). Based on in vitro studies, inhibition of these transporters by Compound 2 has the potential to increase the absorption of co-administered Compound 1. Thus, a drug-drug interaction (DDI) study to assess the potential for Compound 2 to enhance the absorption of Compound 1 via inhibition of intestinal P-gp and BCRP is conducted, as described in Table 7.

TABLE 7
Co-administration of Compound 1 and Compound 2

| | Study Day | | | | | |
|---|---|---|---|---|---|---|
| | 1 | 2-6 | 7-12 | 13 | 14-16 | 17 |
| Cohort 2 | Single- | No | 15 mg | Single-dose | No | Clinic |
| | dose of | Treatment | Compound 2 | of | Treatment | Discharge |
| | up to 90 | | QD PO | up to 90 mg | | |
| | mg | | | Compound 1 + | | |
| | Compound 1 | | | Single dose | | |
| | PO | | | of 15 mg | | |
| | | | | Compound 2 | | |

### Example 4: Food Effect on Compound 1 in Healthy Humans

The food effect on uptake and pharmacokinetics of Compound 1 in healthy participants is determined by administering Compound 1 during the fed and fasted state, as described in Table 8, in combination with PK sampling.

TABLE 8
Food Effect Studies

| Study | Study Day | | | | |
|---|---|---|---|---|---|
| Period | Period 1/Day 1 | Days 2-8 | Period 2/Day 9 | Days 10-11 | Day 12 |
| Cohort 3 | Up to 90 mg | Washout | Up to 90 mg | No | Clinic |
| Sequence | Compound 1 PO | | Compound 1 (Fed) | Treatment | Discharge |
| A (n = 4) | (Fasted) | | | | |
| Cohort 3 | Up to 90 mg | Washout | Up to 90 mg | No | Clinic |
| Sequence | Compound 1 PO | | Compound 1 PO | Treatment | Discharge |
| B (n = 4) | (Fed) | | (Fasted) | | |

Participants undergo an overnight fast (no food or liquids, except water, for at least 10 hours prior to dosing). On Day 9 in Sequence A, and Day 1 in Sequence B, a high-fat/high-calorie breakfast containing ˜1000 kcal and 45% to 55% fat is provided 30 minutes prior to study drug administration. Study drug is administered at or within 5 minutes after participants complete (100% consumption) the breakfast.

Thyroid axis safety monitoring and cardiovascular safety monitoring are conducted as described in the preceding Examples.

### Example 5: Effects of Compound 1 on Mouse NASH Model

C57BL/6J mice were fed a high fat diet for 10 weeks to induce obesity (>38 g BW). Obese mice were injected intraperitoneally (i.p.) twice a week for four weeks with 0.5 μl/g 25% CCl₄ (formulated in olive oil) to induce fibrosis, and one group of normal BW mice were injected i.p. twice a week for four weeks with olive oil to serve as a healthy control. During the same dosing period, obese mice were fed orally once a day for 28 days with vehicle or varying doses of Compound 1. On CCl₄ dosing days, CCl₄ was administered at 4 hours post compound or vehicle dosing. On day 27, all animals were fasted for about 16 hours before terminal euthanasia. On day 28, all animals were sacrificed and various biological parameters were analyzed. Total body, liver, heart and brain weight were measured and changes in liver and heart weight were normalized using brain weight.

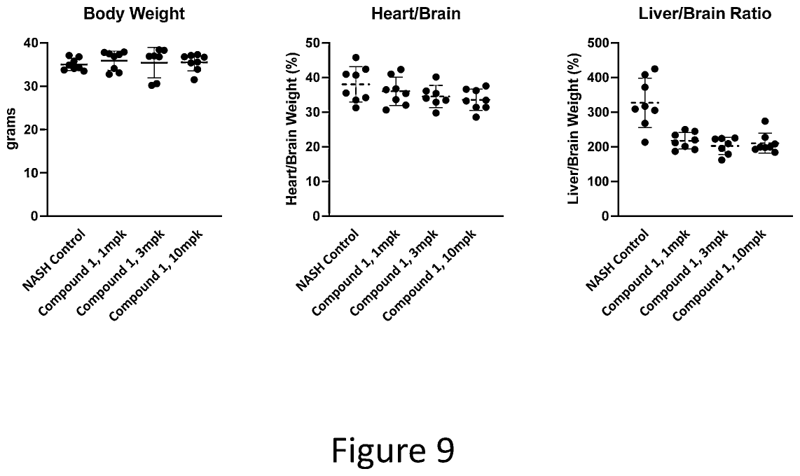

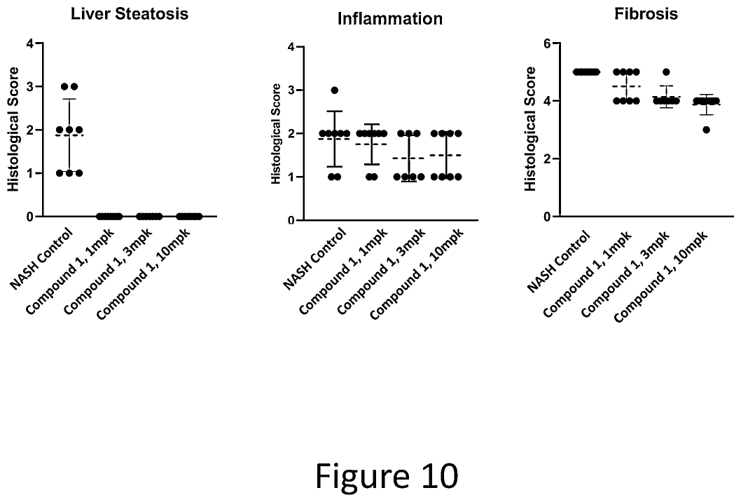

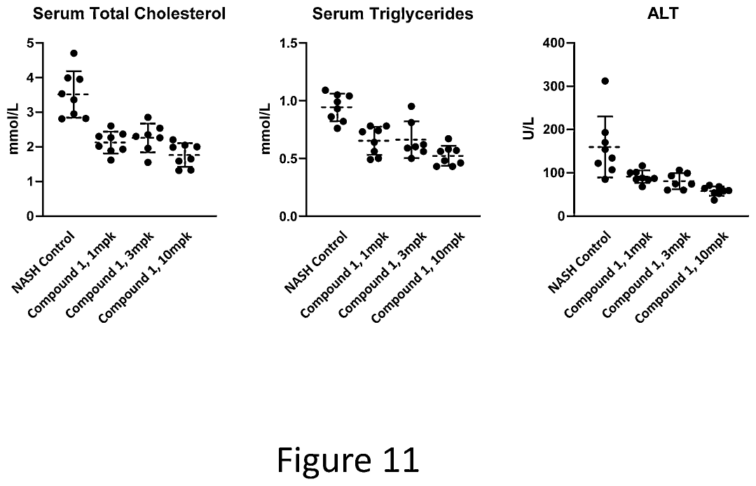

Compound 1 significantly reduced liver/brain weight with no effect on total body weight or heart/brain weight (FIG. 9). Liver tissue histology was analyzed for effects of Compound 1 on steatosis, inflammation and fibrosis. Compound 1 significantly reduced steatosis at all doses tested, showed a trend in inflammation reduction and significantly reduced liver fibrosis at 3 and 10 mpk (FIG. 10). Compound 1 also significantly reduced serum total cholesterol, triglycerides and ALT at all doses tested (FIG. 11).

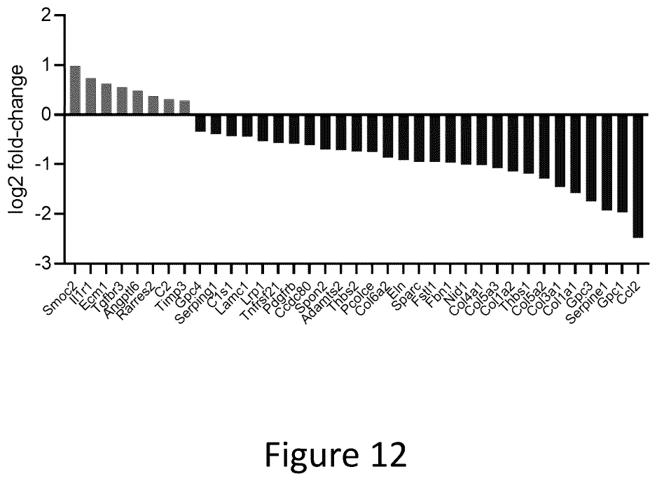

Liver samples were collected for whole transcriptome analysis by RNA sequencing (RNAseq). RNAseq library (n=5 per group) preparation and sequencing was performed using Illumina standard protocols. Alignment of sequencing reads was performed using STAR aligner software and read counts were estimated using RSEM. Differentially expressed genes (compared to vehicle-treated NASH control mice) were determined using EdgeR software. Gene ontology analysis was performed using Advaita software with fold-change and adjusted p-value cutoffs of >1.5 and <0.05, respectively. Gene ontologies were derived from the Gene Ontology Consortium database (2019 Apr. 26) (Ashburner et al., Gene ontology: Tool for the unification of biology. Nature Genetics 25(1): 25-9 (2000); Gene Ontology Consortium, Creating the Gene Ontology Resource: Design and Implementation. Genome Research 11: 1425-1433 (2001)). Compound 1 had a significant effect on expression of genes associated with collagen extracellular matrix and hepatic stellate cell activation, primarily by reducing their expression levels relative to NASH control mice (FIG. 12).

### Example 6: pH and Solubilizer Effect on Compound 1 Solubility

Solubility of Compound 1 (potassium salt) was evaluated at various pH levels. The solubility of Compound 1 in aqueous solution was pH dependent and increased with pH, as shown in Table 9. In the presence of a solubilizer (sodium lauryl sulfate, SLS), the solubility of Compound 1 further improved to 308 μg/mL in pH 10.0 buffer+2 wt % SLS at 25° C. after 24 h.

TABLE 9
pH and solubilizer effect on solubility of Compound
1. (Approximate pKa of compound 1 = 4.12.)

| | Solubility (μg/mL) | |
|---|---|---|
| Solvent | 24 h (without 2 wt % SLS) | 24 h (with 2 wt % SLS) |
| 0.1N HCl | <0.03 | 5.7 |
| pH 2.0 buffer | <0.07 | 5.5 |
| pH 4.0 buffer | <0.07 | 10.0 |
| pH 6.0 buffer | 2.2 | 42.3 |
| pH 8.0 buffer | 10.8 | 301.9 |
| pH 10.0 buffer | 10.2 | 308.2 |

### Example 7: Compound 1 Formulations, Pharmacokinetics, Food Effect in Beagle Dogs

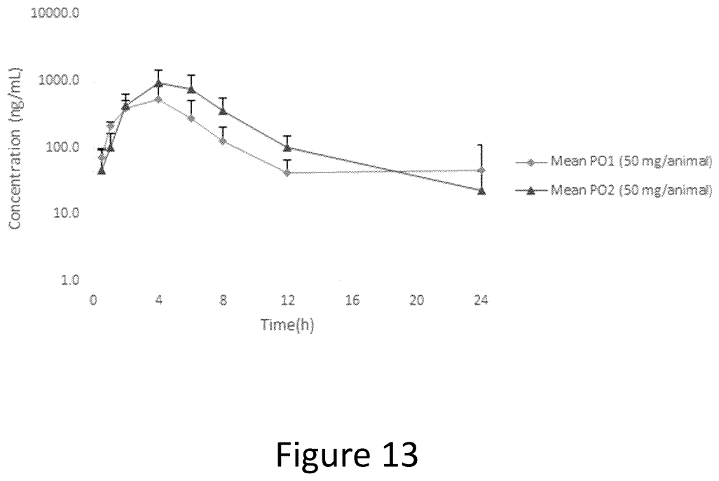

To determine the effect of the solubilizer (SLS) on the uptake of Compound 1, 50 mg (based on free acid) Compound 1 was formulated in a capsule with or without 5 wt % SLS (see Table 10) and was administered to fasted beagle dogs pre-treated with pentagastrin (6 μg/kg, administered by intramuscular injection 30±2 minutes prior to administration of Compound 1). The plasma concentration of Compound 1 was measured over 24 hours (see FIG. 13). Formulation with 5 wt % SLS increased the exposure (Cₘₐₓ, AUC) of Compound 1 by more than 70% (see Table 11).

TABLE 10
Composition of 50 mg Compound 1 capsule formulations.

| | Formulation 1 | Formulation 2 |
|---|---|---|
| | (PO1) (wt %) | (PO2) (wt %) |
| Compound 1 | 18.25 | 18.25 |
| Microcrystalline Cellulose PH-102 | 51.16 | 47.83 |
| Mannitol 200SD | 25.58 | 23.92 |
| Sodium Lauryl Sulfate SLS Fine | 0 | 5.0 |
| Croscarmellose Sodium | 3.0 | 3.0 |
| Colloidal Silica Dioxide | 1.0 | 1.0 |
| Aerosil 200 Pharma | | |
| Magnesium Stearate | 1.0 | 1.0 |
| LIGAMED MF-2-V | | |

TABLE 11
Pharmacokinetics of Compound 1 after dosing
50 mg capsules in beagle dogs (n = 5).

| | Formulation 1 | Formulation 2 |
|---|---|---|
| Cₘₐₓ (ng/mL) | 572 | 1010 |
| Tₘₐₓ (h) | 3.60 | 3.20 |
| t_{1/2} (h) | 4.46 | 4.11 |
| AUC₀₋ₗₐₛₜ (ng*h/mL) | 3420 | 6210 |
| AUC_{0-inf} (ng*h/mL) | 3700 | 6360 |

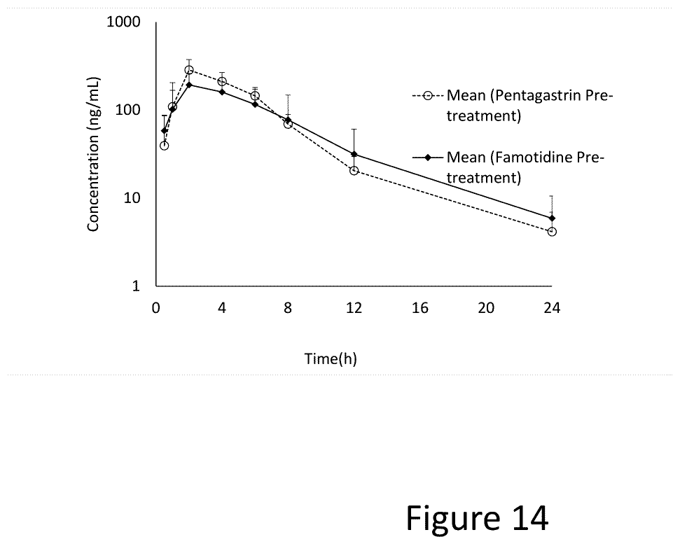

To determine the pH effect on PK performance, beagle dogs were divided into two groups (n=3 per group). For group 1, dogs were pre-treated with pentagastrin (6 μg/kg, intramuscular injection 30±2 minutes prior to administration of Compound 1). For group 2, dogs were pre-treated with famotidine (2 tablets, 20 mg/tablet, oral administration 180±10 minutes prior to administration of Compound 1). Compound 1 (10 mg capsule, 5% SLS) was administered, and plasma concentrations of Compound 1 were monitored for 24 h and are depicted in FIG. 14 Minimal pH effect occurred in dogs under the study conditions.

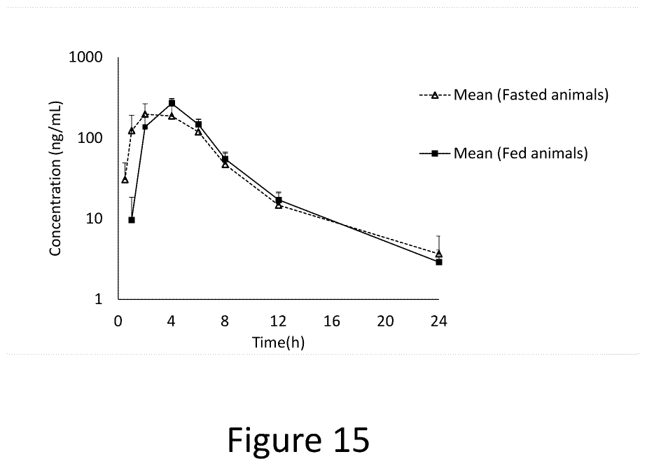

To determine the food effect on PK performance, beagle dogs were divided into two groups (n=3 per group). For the fasted group, dogs were fasted overnight through 4-hours post dosing. For the fed group, dogs were fed high fat food 30 minutes prior to administration. Plasma concentrations of Compound 1 were monitored for 24 h. Compound 1 (10 mg capsule, 5% SLS) was administered, and plasma concentrations of Compound 1 were monitored for 24 h and are depicted in FIG. 15. Food intake delayed Compound 1 Tₘₐₓ but did not have an impact on the plasma exposure.

### Example 8. Production of Potassium Salt of Compound 1

Ethyl (E)-(2-cyano-2-(2-(3,5-dichloro-4-((4-oxo-3,4-dihydrophthalazin-1-yl)oxy)phenyl)hydrazineylidene)acetyl)carbamate (7.4 kg, 0.99-1.01×), potassium acetate (7.4 kg, 0.95-1.00×) and DMAc (41 kg, 5.6-6.0×) were charged into a 500 L GL reactor. The resulting mixture was kept at 80-90° C. for 12-16 h. The mixture was adjusted to 20-30° C. A solution of KOH (0.85 kg, 0.11-0.17×) in process water (8 kg, 1.0-1.5×) was added at 20-30° C. for 1-2 h. The mixture was stirred at 20-30° C. for 1-2 h. Process water (39 kg, 5.0-5.5×) was added at 20-30° C. for 4-6 h. The mixture was stirred at 20-30° C. for 2-3 h. The resulting mixture was centrifuged by a stainless-steel centrifuge. The wet cake was rinsed with process water twice (18+20 kg, 2-3×). Charged the wet cake and process water (38 kg, 5.0-6.0×) into the 500 L GL reactor. The mixture was stirred at 20-30° C. for 2-3 h. The resulting mixture was centrifuged by a stainless-steel centrifuge. The wet cake was rinsed with process water twice (18+22 kg, 2-3×), and dried by a stainless steel dryer under reduced pressure at 55-65° C. to obtain the crude potassium salt of Compound 1 (5.45 kg, purity: 98.4%; assay: 95.9%; yield: 72%).

The crude potassium salt of Compound 1 (5.3 kg, 0.99-1.01×) and DMSO (43 kg, 6.0-8.0×) were charged into a 250 GL reactor (R1). The resulting mixture was kept at 40-50° C. for 0.5-1 h to give a clear solution. Ethyl acetate (EA) (22 kg, 4.0-4.5×) was added at 40-50° C. over 1-2 h. The resulting mixture was filtered through inline filter and transferred into a 250 GL reactor (R2). R1 was rinsed with DMSO (1.8 kg, 0.2-0.5×), and the material was filtered through inline filter and transferred into R2. Adjusted to 40-50° C. and charged EA (22 kg, 4.0-4.5×) into R2 at 40-50° C. over 1-2 h. Charged Compound 1 seeds (0.010 kg, 0.001-0.002×) into R2. The resulting mixture was stirred at 40-50° C. for 1-2 h. Charged EA (67 kg, 12.0-13.0×) into R2 at 40-50° C. over 12-15 h, and stirred at 40-50° C. for 2-3 h. Sampled for analysis (spec: XRPD of wet cake: consistent to reference form A). Charged EA (27 kg, 1.0-5.0×) into R2 at 40-50° C. over 2 h, and stirred at 40-50° C. for 2-3 h. Adjusted R2 to 20-30° C. over 2 h and stirred at 20-30° C. for 4-6 h. The resulting mixture was filtered in a stainless steel filter dryer. The wet cake was rinsed with EA twice (16+14 kg, 2-3×). Charged EA (36 kg, 6-7×) into the stainless steel filter dryer. Adjusted to 20-30° C. and stirred at 20-30° C. for 2-3 h. The resulting mixture was filtered in a stainless steel filter dryer. The wet cake was rinsed with EA (16 kg, 2-3×), and dried by a stainless steel filter dryer under reduced pressure at 60-70° C. The material was sieved to give the purified potassium salt of Compound 1 (4.16 kg, purity: 99.81%; assay: 97.6%; yield: 80%). See FIG. 16.

### Example 9. Salt Screening Study for Compound 1

Several salt forms of Compound 1 were evaluated based on their properties (including solubility, stability and hygroscopicity) relative to the potassium salt form prepared in Example 8 (see FIG. 16, Table 12).

TABLE 12
Peak List: (Form A of potassium salt of Compound 1)

| Pos. | Height | FWHM Left | d-spacing | Rel. Int. |
|---|---|---|---|---|
| [°2θ] | [cts] | [°2θ] | [Å] | [%] |
| 6.16 | 320.90 | 0.1023 | 14.34 | 37.17 |
| 6.78 | 530.15 | 0.1023 | 13.04 | 61.40 |
| 8.49 | 121.75 | 0.1023 | 10.41 | 14.10 |
| 11.35 | 687.96 | 0.1023 | 7.79 | 79.68 |
| 13.51 | 243.61 | 0.1279 | 6.56 | 28.22 |
| 14.44 | 376.23 | 0.1279 | 6.13 | 43.58 |
| 15.76 | 238.73 | 0.1279 | 5.62 | 27.65 |
| 16.81 | 119.97 | 0.2047 | 5.27 | 13.90 |
| 19.77 | 69.22 | 0.2047 | 4.49 | 8.02 |
| 20.51 | 863.38 | 0.1535 | 4.33 | 100.00 |
| 24.63 | 277.93 | 0.1279 | 3.61 | 32.19 |
| 25.97 | 330.30 | 0.1279 | 3.43 | 38.26 |
| 26.68 | 131.82 | 0.1535 | 3.34 | 15.27 |
| 29.13 | 370.67 | 0.1791 | 3.07 | 42.93 |
| 31.00 | 53.89 | 0.3070 | 2.88 | 6.24 |
| 31.75 | 87.04 | 0.1535 | 2.82 | 10.08 |
| 34.09 | 86.07 | 0.1535 | 2.63 | 9.97 |
| 35.67 | 146.38 | 0.2047 | 2.52 | 16.95 |
| 36.47 | 73.59 | 0.2047 | 2.46 | 8.52 |
| 37.74 | 93.93 | 0.1535 | 2.38 | 10.88 |
| 38.90 | 88.02 | 0.2047 | 2.32 | 10.20 |

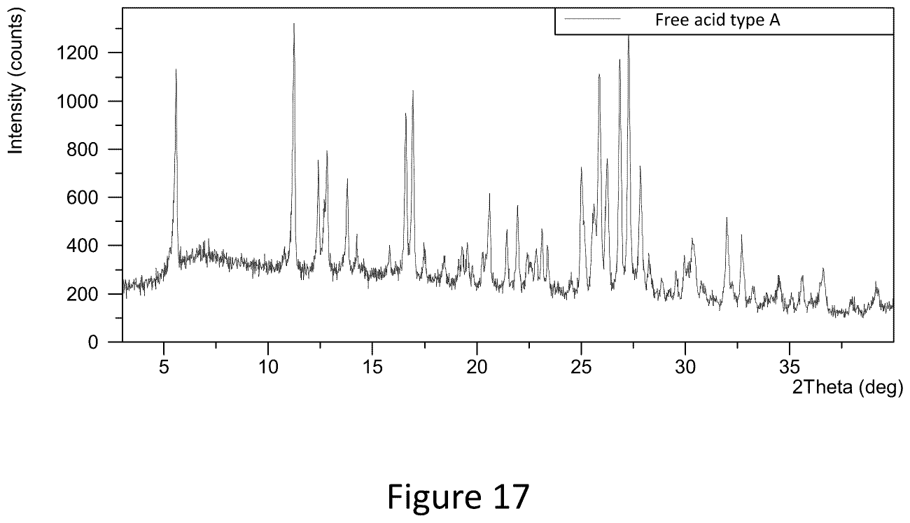

Using the free acid form A (FIG. 17) as the starting material, a total of 50 salt screening experiments were performed with 10 salt formers and 5 solvents. Characterization results showed that a total of 14 salts were obtained in salt screening and re-preparation experiments, including ammonium salt Type B/C1, L-lysine salt Type A/B/C, magnesium salt Type A, choline salt Type A, calcium salt Type A, L-arginine salt Type A/B, sodium salt Type A, Tris salt Type A/B, diethylamine salt Type A and 4 forms of the free acid, including free acid Type A/B/C/D. ¹ According to XRPD comparison, the sample with the name of ammonium salt Type A was speculated as a mixture of freeform Type D and ammonium salt.

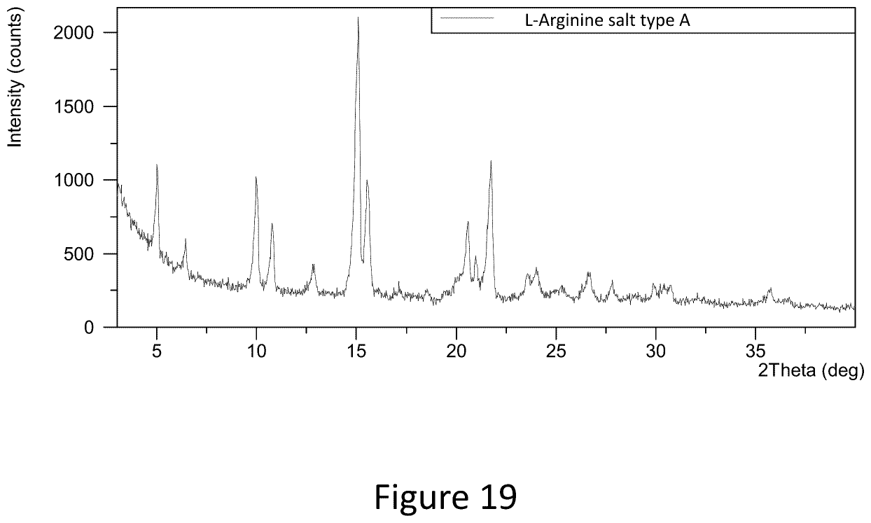

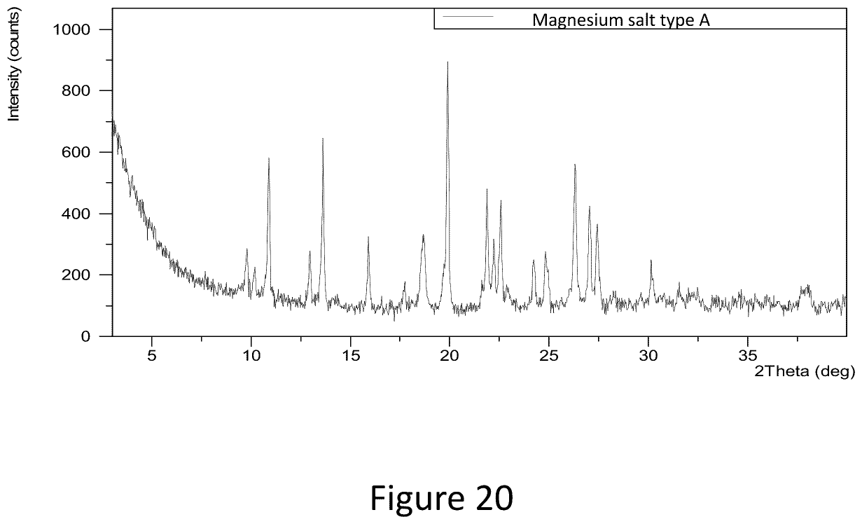

Based on characterization data (using good crystallinity, neat DSC signal, integer molar ratio, high safety class, and simple polymorphism as criteria), sodium salt Type A (FIG. 18, Table 13), L-arginine salt Type A (FIG. 19), and magnesium salt Type A (FIG. 20) were selected for re-preparation and kinetic solubility evaluation, with the potassium salt as a reference in the comparison. The results of the solubility evaluation showed that the sodium salt demonstrated the highest solubility in water at room temperature for 24 hrs (˜0.4 mg/mL, similar to that of potassium salt in water at 37° C.). In simulated gastric fluid (SGF) at 37° C., the potassium salt demonstrated the highest solubility (0.11 mg/mL), followed by sodium salt. In fasted-state simulated intestinal fluid (FaSSIF) at 37° C., the magnesium salt showed the highest solubility (˜0.04 mg/mL), followed by the free acid. In fed-state simulated intestinal fluid (FeSSIF) at 37° C., similar solubility (˜0.02 mg/mL) was observed in the four salts and the free acid. XRPD results showed that except for the free acid, form changes were observed in all the salts in different test media.

TABLE 13
Peak List: (Form A of sodium salt of Compound 1)

| Pos. | Height | FWHM Left | d-spacing | Rel. Int. |
|---|---|---|---|---|
| [°2θ] | [cts] | [°2θ] | [Å] | [%] |
| 5.51 | 1473.10 | 0.1279 | 16.02 | 100.00 |
| 6.99 | 918.64 | 0.1535 | 12.64 | 62.36 |
| 8.47 | 1446.52 | 0.1023 | 10.44 | 98.20 |
| 11.04 | 112.50 | 0.1535 | 8.01 | 7.64 |
| 13.12 | 810.51 | 0.1791 | 6.75 | 55.02 |
| 15.24 | 895.67 | 0.1535 | 5.82 | 60.80 |
| 16.57 | 1066.13 | 0.1279 | 5.35 | 72.37 |
| 17.78 | 357.33 | 0.1023 | 4.99 | 24.26 |
| 20.42 | 502.33 | 0.1279 | 4.35 | 34.10 |
| 21.02 | 397.74 | 0.1791 | 4.23 | 27.00 |
| 22.19 | 353.84 | 0.1023 | 4.01 | 24.02 |
| 23.07 | 113.00 | 0.1535 | 3.86 | 7.67 |
| 25.69 | 114.50 | 0.2303 | 3.47 | 7.77 |
| 27.21 | 264.78 | 0.1023 | 3.28 | 17.97 |
| 27.99 | 70.75 | 0.8187 | 3.19 | 4.80 |
| 28.55 | 551.84 | 0.2047 | 3.13 | 37.46 |
| 29.58 | 102.40 | 0.1535 | 3.02 | 6.95 |
| 31.33 | 465.57 | 0.1535 | 2.86 | 31.60 |
| 32.49 | 52.21 | 0.2047 | 2.76 | 3.54 |
| 33.51 | 90.30 | 0.2047 | 2.67 | 6.13 |
| 34.16 | 93.36 | 0.1535 | 2.62 | 6.34 |

According to the results of kinetic solubility evaluation, with higher solubility (compared with free acid) and simple polymorphism as the criteria, anhydrous sodium salt Type A was selected as a candidate for physicochemical stability evaluation, hygroscopicity and PLM tests, and compared with potassium salt Type A (Table 14). The results of these tests showed:
1) Physicochemical stability evaluation: after storage under 60° C./sealed and 60° C./75% RH for 1 and 2 weeks, minor decrease of HPLC purity (<0.1 area %) and no form change were observed for sodium salt Type A.
2) Hygroscopicity: dynamic vapor sorption (DVS) results showed that sodium salt Type A was slightly hygroscopic with no form change after the DVS test.
3) PLM: sodium salt Type A sample consisted of irregular crystals with particle size <20 μm. The characterization and evaluation data comparison of sodium salt and potassium salt is shown in Table 14.

TABLE 14
Data summary of sodium salt and potassium salt characterization

| | Crystal form | |
|---|---|---|
| Properties | Potassium salt Type A | Sodium salt Type A |
| Crystallinity | High | High |
| TGA loss | 2.1% (up to 300° C.) | 1.6% (up to 180° C.) |
| DSC endothermic signal, ° C. | — | 110.9^{#} |
| (peak) | | |
| HPLC purity, area % | 99.95 | 99.72 |
| Molar ratio (API/former) | 1:1 | 1:1 |
| Speculated form | Anhydrate | Anhydrate |
| Kinetic Solubility | Solubility in water and SGF at | Solubility in water at RT was |
| | 37° C. was higher than that in | higher than that in |
| | FaSSIF/FeSSIF at 37° C., | SGF/FaSSIF/FeSSIF at 37° C., |
| | up to ~0.37 mg/mL | up to ~0.36 mg/mL |
| | (concentration of free acid). | (concentration of free acid). |
| Hygroscopicity/Water uptake | Non-hygroscopic/0.08%/No | Slightly hygroscopic/0.9%/No |
| at 80% RH/Form change after | | |
| DVS | | |

| | |
|---|---|
| Physicochemical stability | After storage under 60° C./sealed and 60° C./75% RH for one and |
| | two weeks, minor decrease of HPLC purity (<0.1 area %) and |
| | no form change were observed |

—: No obvious thermal signal was detected before decomposition (melting point was speculated to be >350° C.).
^{#}the endotherm was speculated to be related with loss of residual solvent or water and the melting point was speculated to be >350° C.

### Example 10. Formulation of Compound 1 Used in Clinical Trials

Compound is an orally bioavailable thyroid hormone receptor beta (THR-β) selective agonist, for treatment of adults with moderate to severe non-cirrhotic nonalcoholic steatohepatitis (NASH). Compound 1 is classified as a BCS Class IV compound and has been formulated as an immediate release capsule. To enhance the intrinsic solubility of the active substance, Compound 1 is isolated as the potassium salt form of the drug substance and sodium lauryl sulfate is used as a solubilizing agent in the immediate release formulation. The formulated product is a dry blend containing between 0.5 to 50 mg Compound 1 (free acid equivalents) in a size 0 HPMC (hydroxypropyl methylcellulose) capsule. In addition to a solubilizer, other classes of excipients such as diluent (such as Microcrystalline Cellulose (MCC), Mannitol etc.), disintegrant (such as Croscarmellose Sodium etc.), glidant (Colloidal Silicon Dioxide etc.), and lubricant (such as Magnesium Stearate etc.) were also investigated and used in the Compound 1 formulation. The quantitative unit dose formulation composition of the active capsules is described in Table 15 below.

TABLE 15
Composition of Compound 1, Capsules, 1 mg, 3 mg, 10 mg, and 50 mg

| | 1 mg | | 3 mg | | 10 mg | | 50 mg | |
|---|---|---|---|---|---|---|---|---|
| | Weight/ | % | Weight/ | % | Weight/ | % | Weight/ | % |
| Component | mg | w/w | mg | w/w | mg | w/w | mg | w/w |
| Compound 1 ^{[1]} | 1.1 | 1.1 | 3.3 | 1.1 | 10.9 | 3.6 | 54.3 | 18.1 |
| Microcrystalline | 59.3 | 59.3 | 177.9 | 59.3 | 172.7 | 57.6 | 143.8 | 47.9 |
| Cellulose | | | | | | | | |
| Mannitol | 29.6 | 29.6 | 88.8 | 29.6 | 86.4 | 28.8 | 71.9 | 24.0 |
| Sodium | 5.0 | 5.0 | 15.0 | 5.0 | 15.0 | 5.0 | 15.0 | 5.0 |
| Lauryl | | | | | | | | |
| Sulfate | | | | | | | | |
| Croscarmellose | 3.0 | 3.0 | 9.0 | 3.0 | 9.0 | 3.0 | 9.0 | 3.0 |
| Sodium | | | | | | | | |
| Colloidal | 1.0 | 1.0 | 3.0 | 1.0 | 3.0 | 1.0 | 3.0 | 1.0 |
| Silicon | | | | | | | | |
| Dioxide | | | | | | | | |
| Magnesium | 1.0 | 1.0 | 3.0 | 1.0 | 3.0 | 1.0 | 3.0 | 1.0 |
| Stearate | | | | | | | | |
| HPMC | 1 capsule | | 1 capsule | | 1 capsule | | 1 capsule | |
| Capsule, Size | | | | | | | | |
| 0 | | | | | | | | |
| Total | 100.0 | 100.0 | 300.0 | 100.0 | 300.0 | 100.0 | 300.0 | 100.0 |

^{[1]} The strengths of 1 mg, 3 mg, 10 mg, and 50 mg are based on free acid. The theoretical content of Compound 1 free acid in potassium salt is 92.1% and the actual amount depends on the assay of drug substance batches used in the drug product.

### Example 11. Compound 1 Direct Encapsulation Formulation Study

A direct encapsulation (DE) formulation study of Compound 1 was carried out. This work included DE process development for the preparation of 1 mg, 3 mg, 10 mg, 50 mg and placebo capsules by a semi-automatic or automatic capsule filling machine. Ingredients, ratios, filling weight, flowability and uniformity of the formulation were all evaluated. The developed processes are suitable for the manufacture of Compound 1 drug products for future clinical studies. Prototype confirmation batch preparation, short term stability studies, GMP confirmation and demonstration stability batch manufacture were conducted to ensure success of the manufacture of GMP batches, and their quality for intended clinical use.

During the initial formulation study of Compound 1, the types of solubilizer were investigated, including solubilizers such as Poloxamer 188 and anionic surfactant sodium lauryl sulfate (SLS). SLS showed good compatibility with Compound 1 and increased the aqueous solubility of Compound 1. The level of SLS between 1.5% to 5.0% w/w was investigated. Pharmacokinetic data indicated that using a certain amount of SLS in the formulation significantly improved bioavailability in vivo. Thus, 1.5% to 5.0% SLS was suitable for further formulation development with Compound 1.

An efficient blending process was developed to meet the challenge of dose uniformity at different dose levels. For 0.5 mg to 10 mg dose level, the equal quantity blending process was developed. Compound 1 was initially blended with the entire amount of SLS and partial amount of MCC by equal quantity. Then the mixture was blended with the remaining excipients, passed through a screen, and then mixed again (a blending-sieving-blending process). By combining Compound 1 together with excipients instead of alone, the yield loss of Compound 1 during the blending process was efficiently reduced. The blending-sieving-blending process effectively incorporated the low-dose Compound 1 with the excipients uniformly in the formulation with higher yields, enabling downstream direct capsule filling or direct tableting. For 10 mg to 50 mg formulations, similar blending and sieving procedures were used in addition to the equal quantity blending process developed and described above. The demonstration batches and GMP batches of each dose strength showed that the blend uniformity results of final blends using this blending process meet the desired product specifications.

The capsule filling weight of formulations containing Compound 1 at different doses and placebos was designed to be 300 mg, because the volume of this material can meet the needs of semi-automatic filling with No. 0 capsules based on the bulk density of the final blends. Semi-automatic filling could be used in the early clinical stage where only small batch sizes of clinical drug products are needed. In addition, a fully automatic capsule filling process has also been developed to meet the large-scale needs of later-stage clinical studies.

Short stability studies under stressed conditions at 60° C./75% RH revealed that a degradation impurity formed for 3 mg strength after two weeks. Silica gel desiccant was subsequently proposed for the packaging and proven to be effective to mitigate the formation of the impurity.

The subsequent manufacture of demonstration stability and GMP batches of 1 mg, 3 mg, 10 mg and 50 mg strength using GMP Compound 1 were conducted according to the final formulation and process. The effectiveness of the silica gel desiccant in minimizing the degradation was evaluated through comparative stability studies with or without desiccant included in the primary packaging. The stability data clearly demonstrated that packaging with desiccant significantly retarded the formation of degradation impurities at 40° C./75% RH condition. Therefore, packaging with 1 g desiccant was determined to improve product quality and increase product shelf life.

### Example 12. Clinical Evaluation of Compound 1 in Healthy Subjects with Elevated LDL-c

Objectives: Assess the overall safety and tolerability of multiple ascending doses of Compound 1 in healthy subjects with elevated LDL-c.

Secondary Objectives: Evaluate the PK and PD of Compound 1 in healthy subjects with elevated LDL-c following multiple ascending doses of Compound 1.

Primary Endpoints: Treatment-emergent adverse events (TEAEs), vital signs, clinical laboratory parameters, and electrocardiogram (ECG) monitoring.

Secondary Endpoints: Plasma PK parameters for Compound 1, PD markers of THR-β agonist target engagement including LDL-c and other lipid parameters and sex hormone binding globulin (SHBG).

Healthy volunteers with mildly elevated LDL-c were randomized 3:1 to Compound 1 (n=6) or placebo (n=2). Volunteers randomized to Compound 1 received multiple doses of 1, 3, 6 or 10 mg of Compound 1 once daily for 14 days in the MAD cohort of the study.

### Results

Compound 1 was generally safe and well-tolerated with a similar incidence of AEs across all Compound 1 treatment groups and placebo. All AEs were mild to moderate with no apparent dose relationship. One placebo subject (1 mg cohort) terminated study early due to withdrawal of consent; all Compound 1 subjects completed study with no premature discontinuations. No study stopping criteria or dose escalation stopping criteria were met.

Liver biochemistry: ALT, AST, ALP and total bilirubin values were overall similar across the treatment groups. No subject receiving Compound 1 had ALT increase to ≥2×ULN. No evidence of DILI. Thyroid hormone: No symptoms of hyper/hypothyroidism. Mean TSH and free T3 values were highly variable but generally similar across the groups. Dose-dependent declines of free T4 were observed among Compound 1 groups consistent with peripheral thyroid hormone modulation observed with other THR-□ agonists. Other laboratory assessments (e.g., clinical chemistry, hematology) showed no apparent trends

Once daily dosing of Compound 1 at 1, 3, 6, and 10 mg for 14 days was overall safe and well-tolerated with no clinical signs or symptoms of hypo/hyperthyroidism or THR-a agonism. Compound 1 exhibited dose-proportional PK with low variability and a half-life suitable for once daily dosing. Compound 1 increased SHBG, a key marker of hepatic THR-β engagement, in a dose-dependent manner. Compound 1 led to significant decreases in circulating atherogenic lipid levels including LDL-c, Apo B, total cholesterol, and triglycerides. Taken together, PD data indicate that administration of Compound 1 led to robust THR-β target engagement in the liver.

### Example 13: Effect of 12 Weeks of Mono- and Combination-Treatments with Compound 2 and Compound 1 on Metabolic Parameters, Hepatic Pathology and NAFLD Activity Score Including Fibrosis Stage in Male Biopsy-Confirmed DIO-NASH Mice

### LIST OF ABBREVIATIONS

Acta2 Actin alpha 2 smooth actin
ALP Alkaline phosphatase
ALT Alanine transaminase
ANOVA Analysis of Variation
AST Aspartate transaminase
BW Body weight
CD146 Melanoma cell adhesion molecule
CDNA Complementary DNA
CK18 M30 Cytokeratin 18 M30
Col1a1 Collagen type I alpha 1
DAB 3,3′-Diaminobenzidine
DEG Differentially Expressed Gene
Dhcr7 7-dehydrocholesterol
DIO Diet-induced obesity
DIO-GAN Diet-induced obesity Gubra Amylin NASH model
ELISA Enzyme-linked immunosorbent assay
FFPE Formalin-fixed paraffin-embedded
FPKM Fragments Per Kilobase of transcript per Million mapped reads
FXR Farnesoid X Receptor
g g-force
Gal-3 Galectin-3
GAN Gubra Amylin NASH
H&E Hematoxylin and Eosin staining
HDL-c High-density lipoprotein cholesterol
Hmgcs1 Hydroxymethylgluteraryl-CoA synthase
HPMC hydroxypropyl methylcellulose
HRP Horseradish peroxidase
IHC Immunohistochemistry
kg Kilogram
LDL-c Low-density lipoprotein cholesterol
Lgals3 Galectin-3
mg Milligram
NA Not applicable
NAFLD Nonalcoholic Fatty Liver Disease
NAS NAFLD Activity Score
NASH Nonalcoholic steatohepatitis
NP-40 Nonidet P-40
PO Per os (oral dosing)
PSR Picrosirius red
QD Once daily

### Introduction

Nonalcoholic steatohepatitis (NASH), a disease manifested by hepatic inflammation and injury in the context of liver steatosis, will likely require combination therapy targeting multiple aspects of the disease to achieve high levels of disease resolution. Small molecule agonists of Farnesoid X Receptor (FXR), a nuclear hormone receptor that maintains homeostasis of metabolic pathways, and thyroid hormone receptor beta (THR-β), a nuclear hormone receptor that regulates metabolic pathways complementary to FXR, are in development for the treatment of NASH. Compound 2, a non-steroidal agonist of FXR, and Compound 1, a liver distributed, selective agonist of THR-β, were evaluated alone and in combination in a diet-induced mouse model of NASH.

### Materials and Methods

### Animal Handling and Study Design

C57BL/6JRj male mice (n=138) were fed the Gubra Amylin NASH (GAN) diet (40% fat, 22% fructose, 2% cholesterol [D09100310, Research Diets]) or lean chow diet for 35 weeks before treatment start. Prior to treatment all animals underwent liver biopsy for histological confirmation (steatosis score ≥2 and fibrosis stage ≥1) and stratification using the nonalcoholic fatty liver disease (NAFLD) activity scoring (NAS) and fibrosis staging system. The diet-induced obese mice on GAN diet (DIO-GAN) were randomized to 8 treatment groups (Table 16) based on percent-area of picrosirius red (PSR) staining. DIO-GAN mice (n=16 per group) received treatment (PO, QD) for 12 weeks with vehicle (0.5% HPMC+0.2% Tween-80 in Tris buffer [50 mM, pH 8]), Compound 2 (10 mg/kg), Compound 1 (0.3 mg/kg [low], 2 mg/kg [med], or 10 mg/kg [high]), or combination treatments of Compound 2 with Compound 1 (Combo-low, Combo-med or Combo-high). Vehicle-dosed lean chow-fed controls served as healthy controls (n=10). Mice were maintained on their respective diets (GAN or lean chow) for the duration of the study. Within-subject comparisons (pre-vs. post-treatment) were performed for liver biopsy histopathological scores. Terminal quantitative endpoints included plasma/liver biochemistry, liver histomorphometry, and liver transcriptomic analysis by RNAseq.

TABLE 16
Treatment Groups

| Group | Dose | | N |
|---|---|---|---|
| Lean Chow vehicle control | NA | | 10 |
| DIO-GAN vehicle control | NA | | 16 |
| Compound 2 | 10 | mg/kg | 16 |
| Compound 1-low | 0.3 | mg/kg | 16 |
| Compound 1-med | 2 | mg/kg | 16 |
| Compound 1-high | 10 | mg/kg | 16 |

| | | |
|---|---|---|
| Combo-low | 10 mg/kg Compound 2 + 0.3 mg/kg | 16 |
| | Compound 1 | |
| Combo-med | 10 mg/kg Compound 2 + 2 mg/kg | 16 |
| | Compound 1 | |
| Combo-high | 10 mg/kg Compound 2 + 10 mg/kg | 16 |
| | Compound 1 | |

Treatment groups assigned to study. DIO-GAN vehicle controls administered vehicle (0.5% HPMC + 0.2% Tween 80 in Tris buffer [pH 8]) once daily. NA, not applicable.

### Liver Biopsy Processing and Scoring

Formalin-fixed paraffin-embedded (FFPE) liver biopsies were prepared by placing liver samples into 10% neutral buffered formalin for ˜24 hours and then transferred to 70% ethanol prior to storage at 4 C. FFPE were placed in the Histokinette to infiltrate prior to embedding in blocks. Biopsy tissues were then cut at 3 μm using a microtome and sections were mounted on slides. Liver sections were stained with Hematoxylin and Eosin (H&E) to assess steatosis, inflammation, and ballooning, and PSR to assess fibrosis. Additionally, slides were processed to detect type I collagen (Col1a1), galectin-3 (Gal-3), and smooth muscle actin (α-SMA) protein expression by immunohistochemistry (IHC). For H&E staining, slides were incubated in Mayer's Hematoxylin (Dako), washed with tap water, stained in Eosin Y solution (Sigma-Aldrich), dehydrated, and coverslipped. For PSR, slides were incubated in Weigert's iron hematoxylin (Sigma-Aldrich), washed in tap water, stained in Picro-sirius red (Sigma-Aldrich), and washed twice in acidified water. Excess water was removed by shaking the slides and the slides were then dehydrated with ethanol, cleared in xylene, and cover-slipped. The NAS and fibrosis stage were scored as described previously (Kleiner et al. 2005). NAS represents the unweighted sum of steatosis, inflammation, and ballooning scores and ranges for 0-8 (Table 17); fibrosis stage ranges from 0 (no fibrosis) to 4 (cirrhosis). For detection of Col1a1, Gal-3 and α-SMA, IHC was performed by standard procedures. Briefly, after antigen retrieval and blocking of endogenous peroxidase activity, slides were incubated with primary antibody (Col1a1: Southern Biotech, Cat. 1310-01; Gal-3: Biolegend, Cat. 125402; α-SMA: Abcam Cat. Ab124964). Primary antibody was detected using a polymeric HRP-linker antibody conjugate. Primary antibody was visualized with DAB as chromogen. Finally, sections were counterstained in hematoxylin and cover-slipped.

TABLE 17
Histological Scoring of NAS and Fibrosis

| Feature | Degree | Score |
|---|---|---|
| Steatosis | <5% | 0 |
| (Percentage of hepatocytes with | 5-33% | 1 |
| lipid droplets) | >33-66% | 2 |
| | >66% | 3 |
| Lobular inflammation | No foci | 0 |
| | <2 foci | 1 |
| | 2-4 foci | 2 |
| Ballooning degeneration | None | 0 |
| | Few | 1 |
| | Many cells/prominent | 2 |
| | ballooning | |
| Fibrosis | None | 0 |
| | Perisinusoidal or periportal | 1 |
| | Perisinusoidal & | 2 |
| | portal/periportal | 3 |
| | Bridging fibrosis | 4 |
| | Cirrhosis | |

### Analysis of Liver Enzymes, Plasma Lipids and CK18 M30

Terminal blood was harvested by cardiac puncture from mice anesthetized with isoflurane (2-3%), mixed with anticoagulant, and placed at 4 C prior to centrifugation at 3000×g for 10 minutes. Plasma supernatants were transferred to new tubes and immediately frozen on dry ice and stored at −80 C. Alanine transaminase (ALT), Aspartate transaminase (AST), Alkaline phosphatase (ALP), Triglycerides (TGs), Total Cholesterol (TC), High-density lipoprotein (HDL-c), and Low-density lipoprotein (LDL-c) were measured using commercial kits (Roche Diagnostics) on the Cobas c 501 autoanalyzer according to manufacturer's instructions. Cytokeratin 18 M30 (CK18 M30) was measured from plasma using a commercial ELISA kit (Cusabio) according to the manufacturer's instructions.

### Analysis of Liver Lipids

Liver samples were homogenized and TGs and TC was extracted in 5% NP-40 by heating (2×) at 90 C. Samples were centrifuged and the TG and TC content was measured in the supernatant using commercial kits (Roche Diagnostics) on the Cobas c 501 autoanalyzer, according to the manufacturer's instructions.

### Liver Transcriptomic Analysis by RNAseq

Tissue was collected and snap-frozen in liquid nitrogen and stored at −80 C until processing. RNA was isolated using a NucleoSpin kit (MACHEREY-NAGEL). A total of 10 ng to 1 μg purified RNA from each sample was used to generate cDNA libraries using the NEBNext Utra II Directional RNA Library Prep Kit for Illumina (New England Biolabs). cDNA libraries were then sequenced on a NextSeq 500 using NextSeq 500/550 High Output Kit V2 (Illumina). Sequencing data was aligned to the mouse genome using the Spliced Transcripts Alignment to a Reference (STAR) software. Differentially expressed genes were identified using the R-package DESeq2.

### Analysis of Trough Plasma Compound Levels

Terminal plasma samples were harvested by cardiac puncture approximately 21-24 hours after the last administration of compound(s). Terminal plasma samples were analyzed by high resolution LC-MS/MS using a Triple Quad 6500+ instrument. 20 μL of plasma sample was mixed with 200 μL of internal standard solution (100 ng/ml Labetalol+100 ng/ml Tolbutamide in acetonitrile), vortexed, and centrifuged at 4,000 rpm for 15 min at 4° C. Supernatant (100 μL) was transferred to a sample plate, mixed with water (100 μL), and shaken (800 rpm) for 10 min prior to injection (2 μL) onto a 1.7 μm, 2.1×50 mm ACQUITY UPLC BEH C18 column (Waters) using a gradient (Table 18) mobile phase of 0.1% formic acid in water (Mobile phase A) and 0.1% formic acid in acetonitrile (Mobile phase B) at a flow rate of 0.6 mL/min. Two internal standards standard 1(retention time: 0.87 min) and standard 2 (retention time: 0.96 min) were used for quantification of Compound 2 (retention time: 0.87 min) and Compound 1 (retention time: 0.97 min), respectively. The internal standard (retention time: 0.76 min), was used for quantification of the glucuronide metabolite of Compound 2 (retention time: 0.77 min). Calibration curves (1-3000 ng/mL) were generated in a matrix of mouse plasma (pooled vehicle control) for each analyte.

TABLE 18
LC-MS/MS Gradient

| Time (min) | Mobile phase B (%) |
|---|---|
| Initial | 35 |
| 1.00 | 98 |
| 1.70 | 98 |
| 1.71 | 35 |
| 2.20 | Stop |

### Results

### Study Design Overview

The diet-induced obese, Gubra Amylin NASH model (DIO-GAN) recapitulates many of the histopathological features of human NASH (Hansen 2020). The DIO-GAN model was used to assess the efficacy of the FXR agonist, Compound 2, and the THR-β agonist, Compound 1, as single agents and in combination. To induce NASH disease, C57BL/6JRj mice were maintained a on diet high in fat, cholesterol, and fructose (GAN diet) for >35 weeks. Prior to therapeutic intervention, mice were biopsied to assess NASH disease and fibrosis severity; mice with a steatosis score <2 and fibrosis stage <1 were excluded from the study. DIO-GAN mice were then randomized into 8 treatment groups (n=16 per group) based on the percent fractional area of Picrosirius red (PSR) staining of the pre-treatment biopsy as well as body fat tissue mass determined by whole body Echo-magnetic resonance imaging (EchoMRI). In the single agent treatment arms, Compound 2 was administered by oral gavage once daily at a dose of 10 mg/kg, while Compound 1 was administered by oral gavage once daily at dose levels of 0.3 (Compound 1-low), 2 (Compound 1-med), or 10 (Compound 1-high) mg/kg. In the combination treatment arms, a constant dose level of Compound 2 (10 mg/kg) was combined with the low, med, and high doses of Compound 1 (i.e., Combo-low, Combo-med, Combo-high, respectively). DIO-GAN mice administered vehicle by oral gavage once daily served as a control. Mice were treated for a total of 12 weeks and were maintained on GAN diet throughout the study. Lean mice (n=10), maintained on normal chow diet throughout the study served as healthy controls.

### Effects of Treatment on Body Weight, Food Intake, and Liver Weight

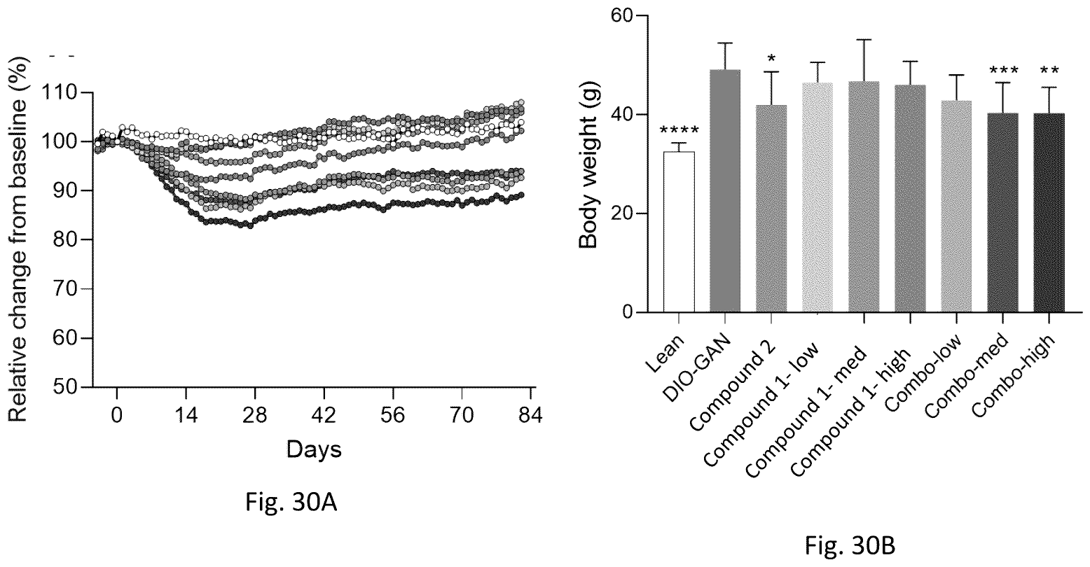

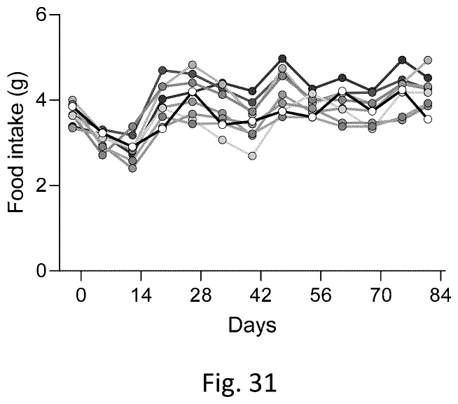

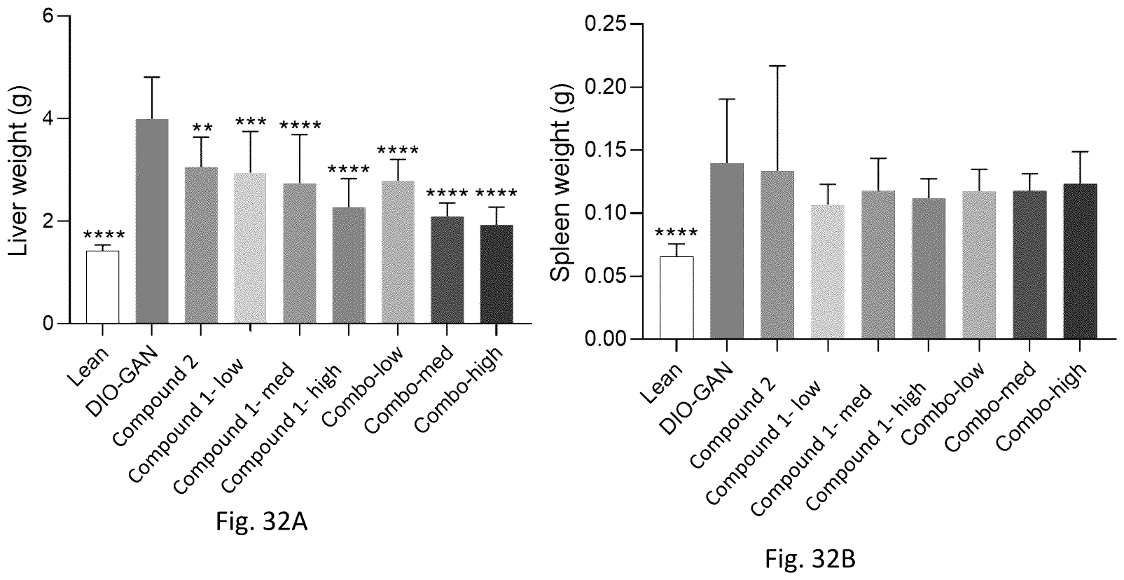

Treatment with Compound 2 alone and in combination with Compound 1 (Combo-low, Combo-med and Combo-high doses) decreased body weight during the study (FIG. 30A). At the end of study, Compound 2, Combo-med and Combo-high treatment groups were significantly lower than the DIO-GAN vehicle control (FIG. 30B). Decreases in body weight did not appear to be associated with decreases in food intake (FIG. 31). All treatment groups significantly improved hepatomegaly (FIG. 32A), with the greatest reductions in liver weight observed in the combination treatment groups (Combo-med and Combo-high); changes in spleen weight were not significant (FIG. 32B).

### Effect of Treatment on Body Mass Composition

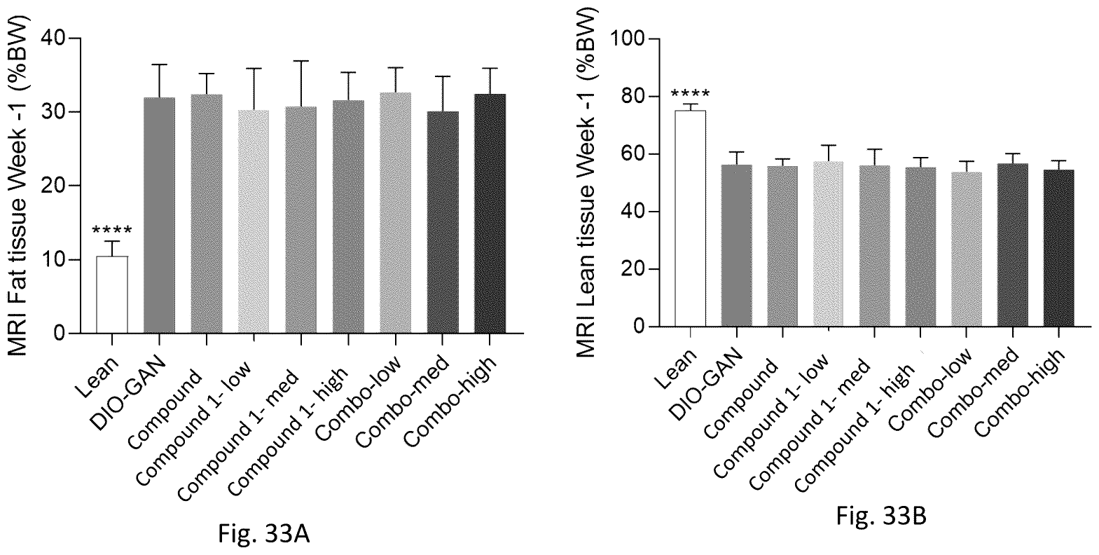

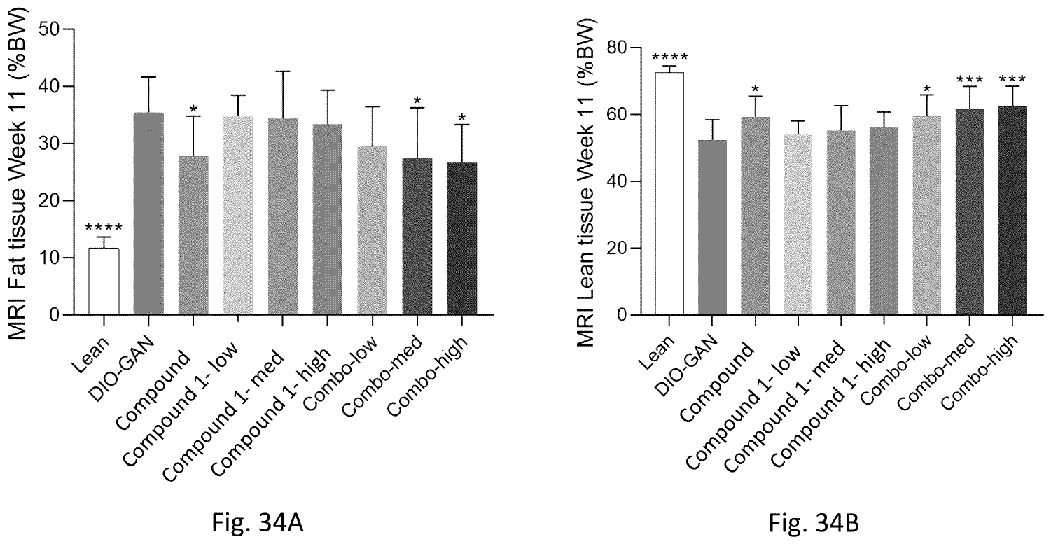

Body composition was determined at baseline (Week-1) and Week 11 of the study by whole body EchoMRI to determine the relative levels lean and fat tissue as a percentage of body weight. Baseline levels of lean and fat tissue were well balanced across treatment groups (FIGS. 33A and 33B). Treatment with Compound 2 and in combination with Compound 1 reduced levels of fat tissue at Week 11 (FIG. 34A); significant increases in relative lean tissue mass were observed in the Compound 2 and combination treatment groups (FIG. 34B).

### Effect of Treatment on Plasma and Liver Lipid Levels

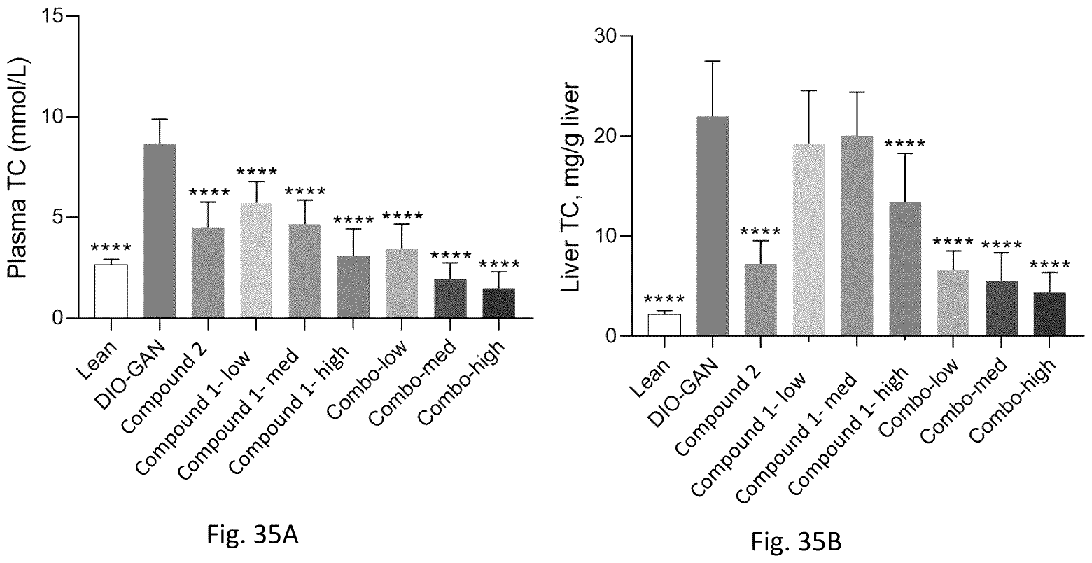

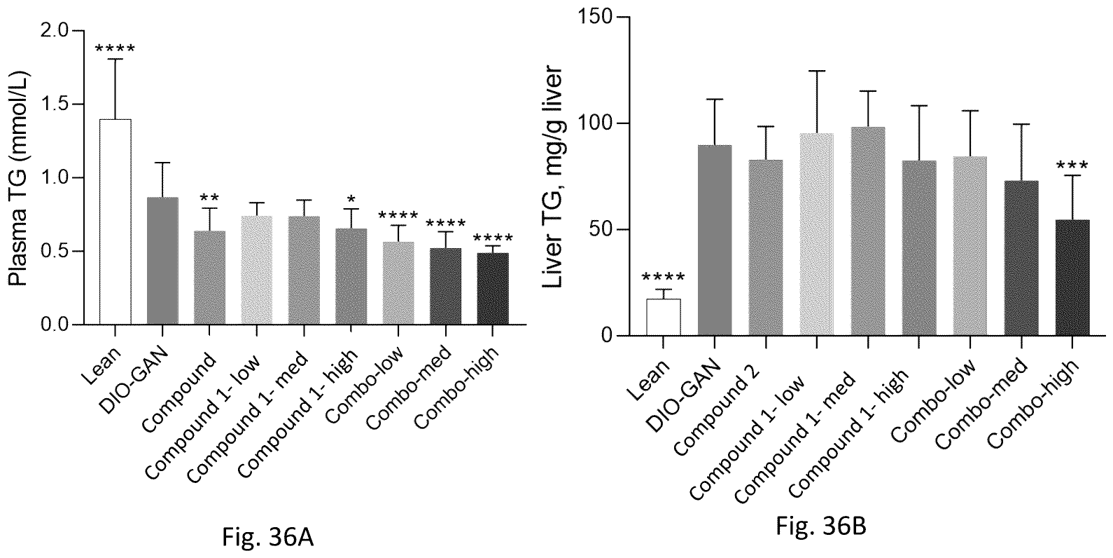

All treatment groups significantly reduced plasma total cholesterol (TC, FIG. 35AA), with the greatest reductions seen with combination treatment (Combo-med and Combo-high); similar trends were observed for TC in the liver (FIG. 35B). Reductions in plasma low-density and high-density lipoprotein cholesterols (HDL-c and LDL-c, respectively) were consistent with the effects seen on TC. Compound 2 and Compound 1-high as well as the combination treatments significantly reduced plasma triglycerides (TG, FIG. 36A); liver TG levels were only significantly reduced in the Combo-high group (FIG. 36B).

### Effects of Treatment on Live Enzymes

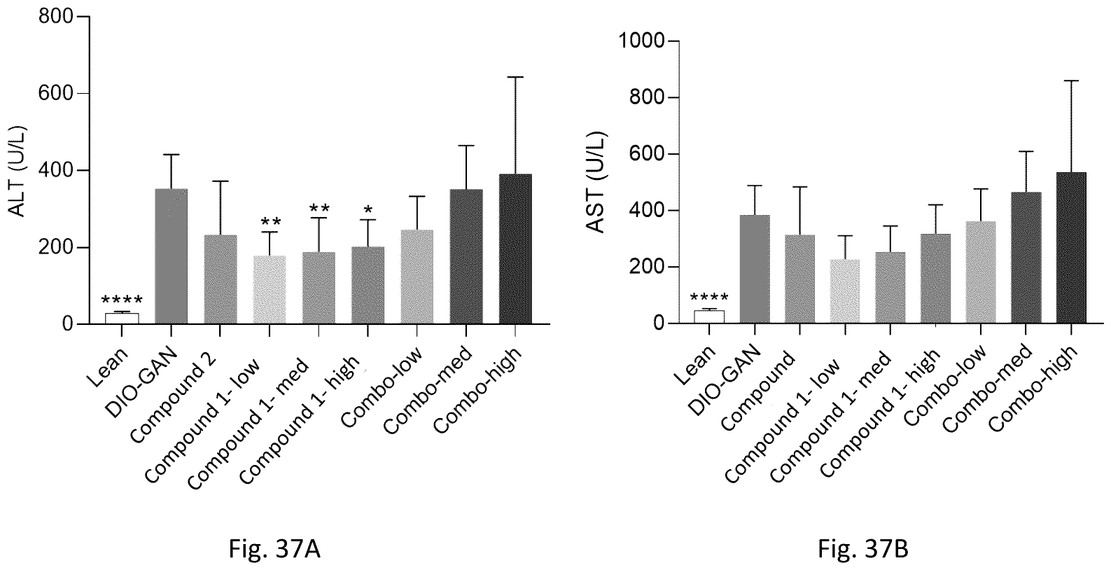

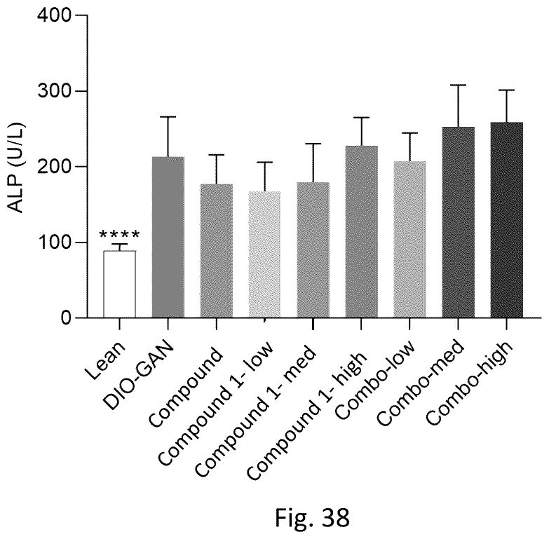

Single agent treatment with Compound 1 (low, med, and high) significantly lowered ALT levels relative to DIO-GAN vehicle control (FIG. 37A); ALT levels were not significantly reduced by combination treatment. AST levels showed a similar trend to ALT although none of the treatment groups were significantly different from the DIO-GAN vehicle control (FIG. 37B). ALP levels were not significantly different from the DIO-GAN vehicle control in any of the treatment groups, although ALP was numerically lower in single agent treatment arms (Compound 2, Compound 1-low and Compound 1-med) and higher in the Combo-med and Combo-high treatments groups relative to DIO-GAN vehicle control (FIG. 38).

### Liver Histology

### NAFLD Activity Score (NAS)

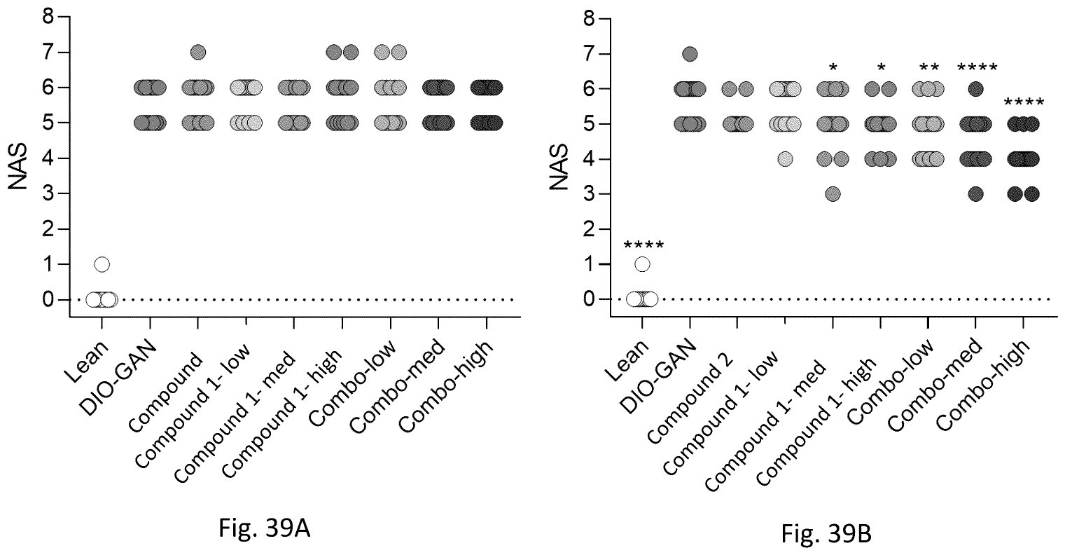

The NAFLD Activity Score (NAS) was used to assess the histological effects of treatment. NAS is defined as the unweighted sum of steatosis, inflammation, and ballooning histological scores and can range from 0-8. NAS was determined for each animal before (baseline) and after 12-weeks of treatment at the end of study. NAS was well balanced across treatment groups with an NAS range of 5-6 at baseline in most mice (FIG. 39A). After 12-weeks of treatment, NAS was significantly improved in most treatment groups with the most profound improvements seen with combination treatment (FIG. 39B). In the Compound 2 treatment group, 56% of the animals showed a ≥1-pt NAS improvement, compared to 31%, 27%, and 62% in the Compound 1-low, Compound 1-med, and Compound 1-high treatment groups, respectively (Table 19). Combination treatment was more effective, with 69%, 81%, and 100% of mice showing ≥1-pt NAS improvement in the Combo-low, Combo-med, and Combo-high combination treatment groups, respectively. In addition, the magnitude of NAS improvement was greater in the combination treatment groups. While 0% of mice in the Compound 2 treatment group improved NAS by >1-pt, 19%, 25%, and 43% of mice in the Combo-low, Combo-med, and Combo-high combination arms, respectively, achieved ≥2-pt NAS improvement. These results were superior to the Compound 1 single agent treatment arms, in which 0%, 7%, and 25% of mice achieved ≥2-pt NAS improvement in the Compound 1-low, Compound 1-med, and Compound 1-high dose groups, respectively.

TABLE 19
Effects of Treatment on NAFLD Activity Score (NAS)

| | | | Worsening | | Improving | | | | | |
|---|---|---|---|---|---|---|---|---|---|---|
| | No change | | % (n) | | % (n) | | | | | |
| Treatment (n) | % (n) | | ≥1-pt | | 1-pt | | ≥2-pt | | Total | |
| Lean Control (10) | 80% | (8) | 10% | (1) | 10% | (1) | 0 | | 10% | (1) |
| DIO-GAN Control | 68.75% | (11) | 25% | (4) | 6.25% | (1) | 0 | | 6.25% | (1) |
| (16) | | | | | | | | | | |
| Compound 2 (16) | 43.7% | (7) | 0 | | 56.25% | (9) | 0 | | 56.25% | (9) |
| Compound 1-low | 56.25% | (9) | 12.5% | (2) | 31.25% | (5) | 0 | | 31.25% | (5) |
| (16) | | | | | | | | | | |
| Compound 1-med | 73.33% | (11) | 0 | | 20% | (3) | 6.7% | (1) | 26.7% | (4) |
| (15) | | | | | | | | | | |
| Compound 1-high | 31.25% | (5) | 6.25% | (1) | 37.5% | (6) | 25% | (4) | 62.5% | (10) |
| (16) | | | | | | | | | | |
| Combo-low (16) | 18.75% | (3) | 12.5% | (2) | 50% | (8) | 18.75% | (3) | 68.75% | (11) |

| | | | | | | | | | |
|---|---|---|---|---|---|---|---|---|---|
| Combo-med (16) | 18.75% | (3) | 0 | 56.25% | (9) | 25% | (4) | 81.25% | (13) |

| | | | | | | | | |
|---|---|---|---|---|---|---|---|---|
| Combo-high (14) | 0 | 0 | 57.1% | (8) | 42.9% | (6) | 100% | (14) |

### Steatosis

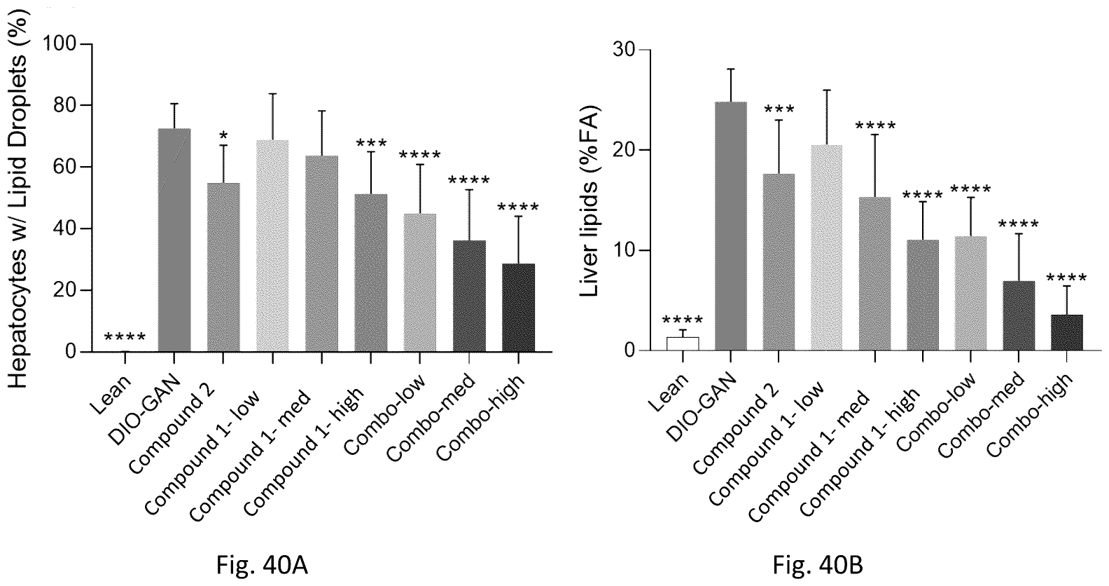

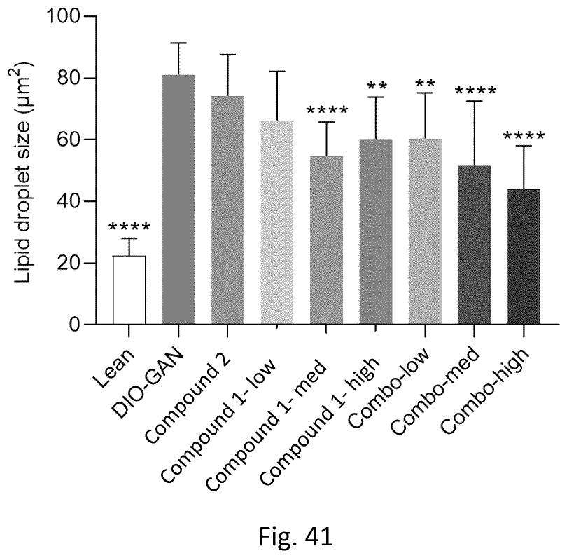

NAS improvements were largely driven by greater reductions in steatosis (Table 20). In the Compound 2 group, 81% showed improved steatosis at the end of treatment, although the maximum improvement was 1-pt. In the Compound 1 single agent treatment groups, dose-dependent increases in the percentage of mice showing steatosis improvement were seen, corresponding to 31%, 47%, and 81% of mice in the Compound 1-low, Compound 1-med, and Compound 1-high dose groups, respectively. In each Compound 1 single agent treatment group one mouse (i.e., ˜6%) showed a ≥2-pt improvement in steatosis. In contrast, 25%, 31%, and 71% of mice in the Combo-low, Combo-med, and Combo-high treatment groups showed ≥2-pt steatosis improvement, respectively. These effects were supported by quantitative liver histomorphometry, which showed a reduced percentage of hepatocytes containing lipid droplets (FIG. 40A) and lower levels of liver lipids (FIG. 40B), as well as smaller lipid droplet size (FIG. 41).

TABLE 20
Effects of Treatment on Histological Steatosis Score

| | | | Improving | | | | | |
|---|---|---|---|---|---|---|---|---|
| | No change | | % (n) | | | | | |
| Treatment (n) | % (n) | | 1-pt | | ≥2-pt | | Total | |
| Lean Control (10) | 100% | (10) | 0 | | 0 | | 0 | |
| DIO-GAN Control | 81.25% | (13) | 18.75% | (3) | 0 | | 18.75% | (3) |
| (16) | | | | | | | | |
| Compound 2 (16) | 18.75% | (3) | 81.25% | (13) | 0 | | 81.25% | (13) |
| Compound 1 Low | 68.75% | (11) | 25% | (4) | 6.25% | (1) | 31.25% | (5) |
| (16) | | | | | | | | |
| Compound 1 Med | 53.33% | (8) | 40% | (6) | 6.7% | (1) | 46.7% | (7) |
| (15) | | | | | | | | |
| Compound 1 High | 18.75% | (3) | 75% | (12) | 6.25% | (1) | 81.25% | (13) |
| (16) | | | | | | | | |
| Combo Low (16) | 6.25% | (1) | 68.75% | (11) | 25% | (4) | 93.75% | (15) |
| Combo Med (16) | 6.25% | (1) | 62.5% | (10) | 31.25% | (5) | 93.75% | (15) |

| | | | | | | | |
|---|---|---|---|---|---|---|---|
| Combo High (14) | 0 | 28.6% | (4) | 72.4% | (10) | 100% | (14) |

The combination of Compound 2 and Compound 1 resulted in greater improvements in liver steatosis. Liver steatosis was determined by histology at baseline and end of treatment for each individual mouse. Table 29 shows the percentage of mice within each treatment group with no change or improving (1-pt and >2-pt decrease from baseline) steatosis score. Total represents the percentage of mice in each treatment group showing at least 1-pt steatosis improvement from baseline.

### Ballooning, Inflammation, and Fibrosis

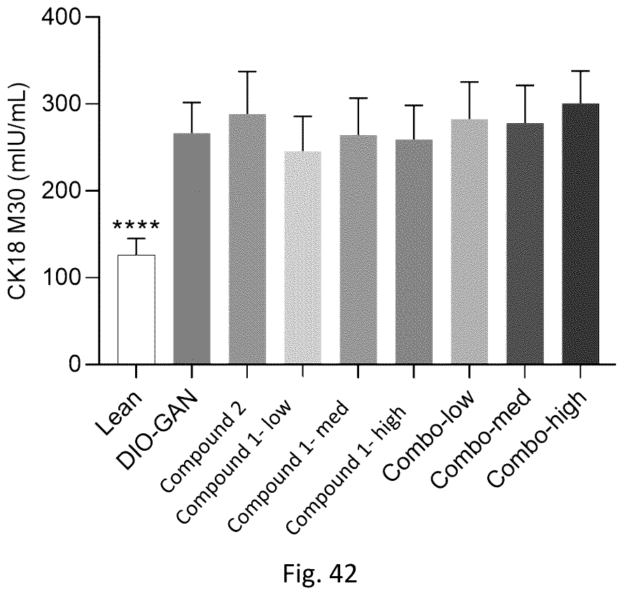

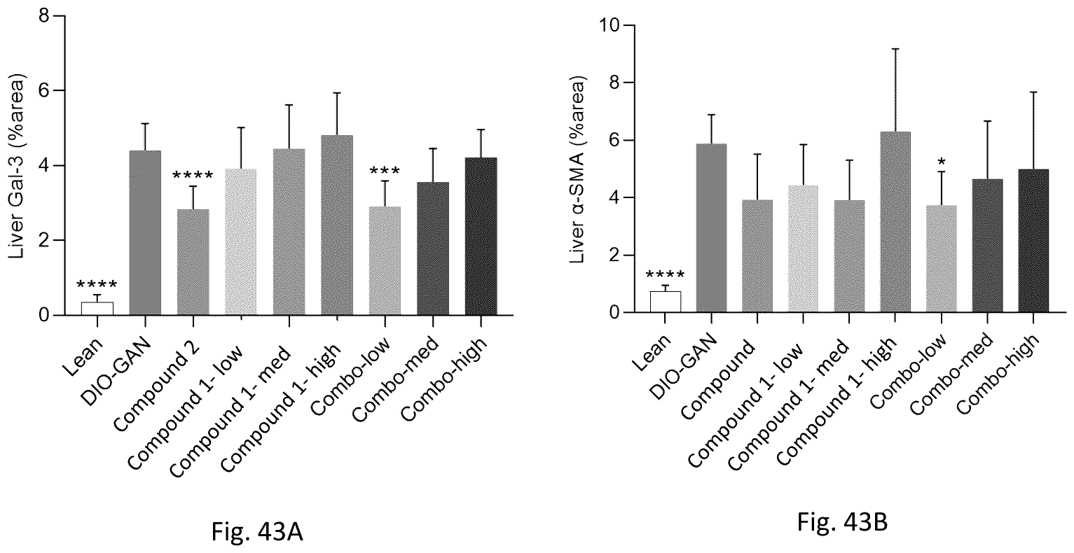

Hepatocellular ballooning, an indicator of apoptosis, was infrequently observed and not significantly changed by any of the treatments. CK18 M30, a plasma biomarker associated with apoptosis, was also not significantly different between treatment groups and the DIO-GAN vehicle control (FIG. 42). Lobular inflammation was not significantly improved by treatment, although improvements in inflammation scores were observed, albeit infrequently, in the Compound 1-low, Compound 1-med, and Compound 1-high treatment groups as well as in the Combo-med combination group (Table 21). To further assess inflammation, protein expression Galectin-3 (Gal-3), a marker of inflammatory lymphocyte infiltration, was determined by immunohistochemistry (IHC) staining of the liver. Treatment with Compound 2 alone and in combination with Compound 1 (Combo-low) resulted in lower levels of Gal-3 expression in the liver relative to DIO-GAN vehicle control (FIG. 43A).

TABLE 21
Effects of Treatment on Lobular Inflammation

| | Worsening | | No change | | Improving | |
|---|---|---|---|---|---|---|
| Treatment (n) | % (n) | | % (n) | | % (n) | |
| Lean Control (10) | 10% | (1) | 80% | (8) | 10% | (1) |
| DIO-GAN Control | 31.25% | (5) | 68.75% | (11) | 0 | |
| (16) | | | | | | |
| Compound 2 (16) | 31.25% | (5) | 68.75% | (11) | 0 | |
| Compound 1 Low | 25% | (4) | 68.75% | (11) | 6.25% | (1) |
| (16) | | | | | | |
| Compound 1 Med | 20% | (3) | 73.3% | (11) | 6.7% | (1) |
| (15) | | | | | | |
| Compound 1 High | 31.25% | (5) | 56.25% | (9) | 12.5% | (2) |
| (16) | | | | | | |
| Combo Low (16) | 31.25% | (5) | 68.75% | (11) | 0 | |
| Combo Med (16) | 31.25% | (5) | 62.5% | (10) | 6.25% | (1) |

| | | | | | |
|---|---|---|---|---|---|
| Combo High (14) | 35.7% | (5) | 64.3% | (9) | 0 |

Inflammation was not significantly improved by treatment. Lobular inflammation was determined by histology at baseline and end of treatment. Table 30 shows the percentage of mice within each treatment group with worsening (≥1-pt increase from baseline), no change, or improving (≥1-pt decrease from baseline) lobular inflammation scores.

Improvements in fibrosis stage were more frequently observed in the combination agent treatment groups compared with the single agent arms, although differences did not reach significance (Table 22). Changes in Col1al protein expression, as determined by IHC staining of the liver, were not significantly different between treatment groups. Numeric reductions in α-SMA, a marker of hepatic stellate cell activation, were observed in the Compound 2, Compound 1-low, and Compound 1-med treatment group. Significant reductions were only observed in the Combo-low treatment group (FIG. 43B).

TABLE 22
Effects of Treatment on Liver Fibrosis

| | Worsening | | No change | | Improving | |
|---|---|---|---|---|---|---|
| Treatment (n) | % (n) | | % (n) | | % (n) | |
| Lean Control (10) | 0 | | 100% | (10) | 0 | |
| DIO-GAN Control | 31.25% | (5) | 68.75% | (11) | 0 | |
| (16) | | | | | | |
| Compound 2 (16) | 37.5% | (6) | 56.25% | (9) | 6.25% | (1) |
| Compound 1 Low | 31.25% | (5) | 62.5% | (10) | 6.25% | (1) |
| (16) | | | | | | |
| Compound 1 Med | 26.7% | (4) | 60% | (9) | 13.33% | (2) |
| (15) | | | | | | |
| Compound 1 High | 25% | (4) | 68.75% | (11) | 6.25% | (1) |
| (16) | | | | | | |
| Combo Low (16) | 37.5% | (6) | 43.75% | (7) | 18.75 | (3) |
| Combo Med (16) | 18.75% | (3) | 75% | (12) | 6.25% | (1) |
| Combo High (14) | 21.4% | (3) | 50% | (7) | 28.6% | (4) |

Fibrosis improvement more frequently observed with combination treatment. Liver fibrosis stage was determined by histology at baseline and end of treatment. Table 31 shows the percentage of mice within each treatment group with worsening (≥1-stage increase from baseline), no change, or improving (≥1-stage decrease from baseline) fibrosis.

### Liver Transcriptomics Analysis by RNAseq

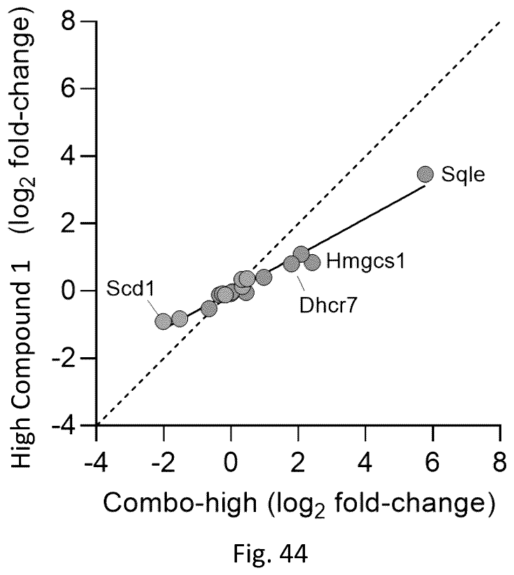

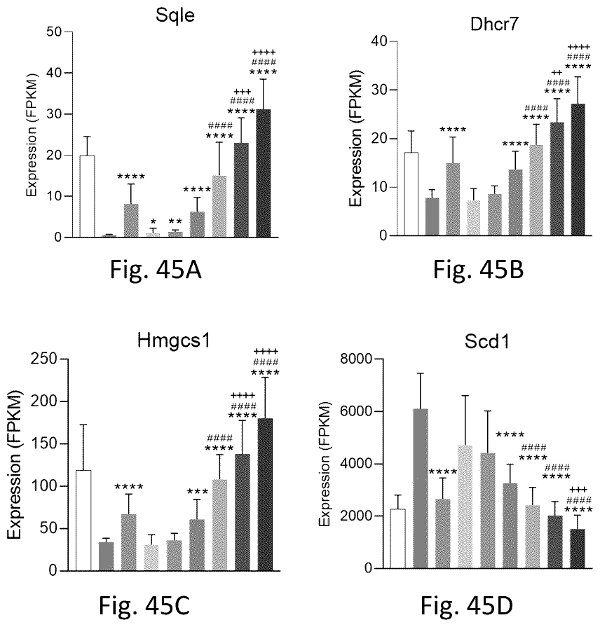

Terminal liver samples (n=10) from treatment groups were processed for transcriptomics analysis by RNAseq. Differentially expressed genes (DEGs) were identified compared to DIO-GAN vehicle control. DEGs were identified in all treatment groups; fewest in Compound 1-low (987) and the largest number of DEGs in Combo-high (3533) treatment group. Comparison of Compound 1-high to Combo-high indicated that genes involved in energy and lipid metabolism were differentially expressed to a greater extent by combination treatment (FIG. 44). This is indicated by comparison of the slopes for the line of identity (dotted line, slope=1) to the linear regression line (solid line, slope=0.55) of fold-change values. Squalene epoxidase (Sqle) and 7-dehydrocholesterol reductase (Dhcr7), enzymes involved in cholesterol metabolism were expressed at significantly higher levels in the combination groups relative to single agent treatment (FIGS. 45A and 45B). Hydroxymethylglutaryl-CoA synthase (Hmgcs1), a key enzyme in energy metabolism, showed a similar pattern of expression (FIG. 45C). Stearoyl-CoA desaturase (Scd1), an enzyme involved in fatty acid metabolism, was reduced by Compound 2 treatment, and further reduced by combination treatment (FIG. 45D).

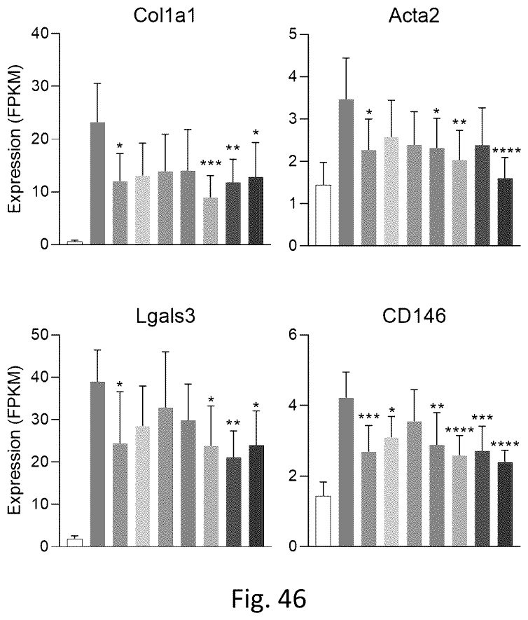

Lastly, we examined the expression of select genes associated with fibrosis and inflammation including collagen type I alpha 1 (Col1a1), actin alpha 2 smooth actin (Acta2), Galectin 3 (Lgals3), and melanoma cell adhesion molecule (CD146). In general, Compound 2 alone and in combination with Compound 1 significantly reduced expression of these genes relative to DIO-GAN vehicle control (FIG. 46); combination treatment arms were not significantly different from Compound 2 treatment alone.

### Summary

The mouse diet-induced obese Gubra-Amylin NASH (DIO-GAN) model was used to evaluate the efficacy of Compound 2 and Compound 1 as single agents and in combination on metabolic and histopathological parameters of NASH and fibrosis. This model has been extensively characterized and recapitulates many aspects of human NASH (Hansen 2020) without the use of hepatotoxic agents to induce disease. In this model, mice were maintained on a diet high in fat, cholesterol, and fructose (GAN diet) for >35 weeks. Prior to therapeutic intervention, mice were biopsied to assess NAFLD activity score (NAS) and fibrosis severity by histology; only mice with a baseline steatosis score of >2 and fibrosis stage >1 were used in the study. Importantly, this preselection step ensures that only mice with significant NAFLD activity were used in the study. In addition, knowledge of the baseline NAS allows for therapeutic responses to be evaluated not only relative to the DIO-GAN vehicle control but also relative to individual baseline values. DIO-GAN mice were treated with Compound 2 and Compound 1 alone and in combination for 12 weeks and maintained on the GAN diet throughout the study. Mice were treated with a single dose level of Compound 2, alone or in combination with 3 dose levels (low [0.3 mg/kg], med [2 mg/kg], and high [10 mg/kg]) of Compound 1 in order to maximize the ability to discern potential additive therapeutic effects.

The combination of Compound 2 and Compound 1 showed greater reductions in NAS relative to single agent treatments both in terms of the percentage of mice showing reduction in NAS and the magnitude of NAS improvement. Improvements in NAS were largely driven by greater reductions in steatosis, which was associated with larger reductions in plasma and liver total cholesterol and triglycerides. The greater overall effects seen with the combination treatment did not appear to be driven by higher exposure of the individual drugs in the combination treatment groups (Table 23). In addition, although changes in body weight may have contributed to NAS improvements, body weight reductions were similar between Compound 2 and combination treatments groups (Combo-low and Combo-med), suggesting that weight loss alone does not fully explain the greater anti-steatotic activity of the combination treatment. Instead, combination treatment had greater effects on the expression of genes related to energy and lipid metabolism. These results suggest that the combination of Compound 2 and Compound 1 appear to have at least an additive effect on these pathways and likely responsible for the greater anti-steatotic activity observed.

Histological improvement in inflammation and fibrosis were not significantly improved by treatment. Evidence of fibrosis improvement was noted, however, including a larger number of mice showing fibrosis improvement with the combination. In addition, transcriptomics analysis identified key markers of fibrosis and inflammation that were reduced by combination treatment. Expression of these genes was also reduced by Compound 2 treatment, suggesting that FXR agonism may be the main driver for effect on fibrosis and inflammation at the gene expression level. In this case, FXR agonism can be considered complementary to the more antisteatotic mechanism of THR-β agonism. Together with the greater antisteatotic effects observed by combining Compound 2 and Compound 1, these results suggest that this combination could address multiple aspects of NASH disease.

### REFERENCES

Kleiner D E, Brunt E M, Van Natta M, Behling C, Contos M J, Cummings O W, Ferrell L D, Liu Y C, Torbenson M S, Unalp-Arida A, Yeh M, Mccullough A J, Sanyal A J; Nonalcoholic Steatohepatitis Clinical Research Network. Design and validation of a histological scoring system for nonalcoholic fatty liver disease. Hepatology. 2005 June; 41(6): 1313-21.
Hansen, H. H., Ægidius, H. M., Oró, D. et al. Human translatability of the GAN diet-induced obese mouse model of non-alcoholic steatohepatitis. BMC Gastroenterol 20, 210 (2020). Supplemental

TABLE 23
Trough plasma drug concentrations determined by LC-MS/MS

| | Mean trough analyte concentration (SD), ng/mL | | | |
|---|---|---|---|---|
| | | Compound 2 | | |
| | | glucuronide | | |
| Treatment Group (n) | Compound 2 | metabolite | Compound 1 | |
| Compound 2 (16) | 7.4 (3.8) | 52.7 (26.0) | ND | |
| Compound 1-med | ND | ND | 135.5 | (62.0) |
| (15) | | | | |
| Compound 1-high | ND | ND | 320.0 | (189.9) |
| (16) | | | | |
| Combo-med (16) | 5.8 (3.5) | 53.1 (36.8) | 49.5 | (42.9) |
| Combo-high (14) | 3.3 (1.8) | 49.0 (23.7) | 143.0 | (87.9) |

Terminal plasma samples collected by cardiac puncture 21-24 hours post final treatment dose (i.e., trough) were analyzed by LC-MS/MS. Values represent mean and standard deviation (SD). ND, not determined.

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention.

What is claimed is:

1. A method of treating non-alcoholic steatohepatitis (NASH) in a patient in need thereof, comprising orally administering to the patient a compound of formula: 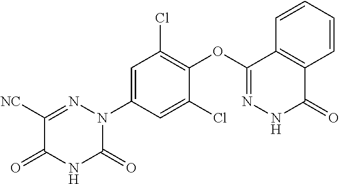 [SMILES: N#Cc1nn(-c2cc(Cl)c(Oc3n[nH]c(=O)c4ccccc34)c(Cl)c2)c(=O)[nH]c1=O]
or a pharmaceutically acceptable salt thereof once daily at a dose of from about 0.5 mg to about 25 mg.

2. The method of claim 1, wherein the compound, or a pharmaceutically acceptable salt thereof, is administered once daily at a dose of from about 1 mg to about 15 mg.

3. The method of claim 1, wherein the compound, or a pharmaceutically acceptable salt thereof, is administered once daily at a dose of from about 2 mg to about 10 mg.

4. The method of claim 1, wherein the compound, or a pharmaceutically acceptable salt thereof, is administered once daily at a dose of about 1 mg.

5. The method of claim 1, wherein the compound, or a pharmaceutically acceptable salt thereof, is administered once daily at a dose of about 3 mg.

6. The method of claim 1, wherein the compound, or a pharmaceutically acceptable salt thereof, is administered once daily at a dose of about 6 mg.

7. The method of claim 1, wherein the compound, or a pharmaceutically acceptable salt thereof, is administered once daily at a dose of about 10 mg.

8. A method of treating non-alcoholic steatohepatitis (NASH) in a patient in need thereof, comprising orally administering to the patient a compound of formula: 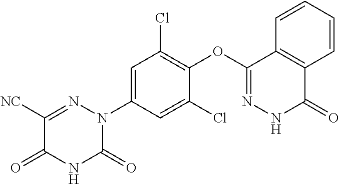 [SMILES: N#Cc1nn(-c2cc(Cl)c(Oc3n[nH]c(=O)c4ccccc34)c(Cl)c2)c(=O)[nH]c1=O]
or a pharmaceutically acceptable salt thereof to obtain a steady state area under the curve from zero to infinity (AUC_{0-∞}) of from about 2,500 ng*h/mL to about 50,000 ng*h/mL.

9. The method of claim 8, wherein the compound, or a pharmaceutically acceptable salt thereof, is administered at a dose to obtain a steady state AUC_{0-∞} of from about 5,000 ng*h/mL to about 50,000 ng*h/mL.

10. The method of claim 8, wherein the compound, or a pharmaceutically acceptable salt thereof, is administered at a dose to obtain a steady state AUC_{0-∞} of from about 5,000 ng*h/mL to about 25,000 ng*h/mL.

11. The method of any one of claims 8 to 10, wherein the compound is administered orally once daily.

12. The method of any one of claims 1 to 11, wherein the compound is administered as a potassium salt.

13. The method of any one of claims 1 to 11, wherein the compound is administered as a sodium salt.

14. The method of any one of claims 1 to 13, wherein the compound is administered in a pharmaceutical composition comprising an ionic surfactant.

15. The method of claim 14, wherein the ionic surfactant is sodium lauryl sulfate.

16. The method of claim 15, wherein the sodium lauryl sulfate is present in the composition at from about 1% to about 8% by weight.

17. The method of claim 15, wherein the sodium lauryl sulfate is present in the composition at from about 1% to about 5% by weight.

18. The method of claim 15, wherein the sodium lauryl sulfate is present in the composition at about 5% by weight.

19. A crystalline potassium salt of a compound of formula:

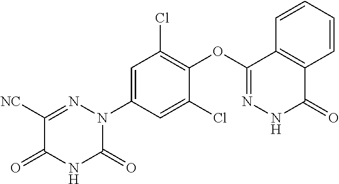 [SMILES: N#Cc1nn(-c2cc(Cl)c(Oc3n[nH]c(=O)c4ccccc34)c(Cl)c2)c(=O)[nH]c1=O]

20. The crystalline potassium salt of claim 19 produced by the method of example 8.

21. A pharmaceutical composition comprising the compound of claim 19 or claim 20.

22. The pharmaceutical composition of claim 21, wherein the amount of the compound in the pharmaceutical composition is from about 1 mg to about 15 mg.

23. The pharmaceutical composition of claim 21, wherein the amount of the compound in the pharmaceutical composition is from about 2 mg to about 10 mg.

24. The pharmaceutical composition of claim 21, wherein the amount of the compound in the pharmaceutical composition is about 1 mg.

25. The pharmaceutical composition of claim 21, wherein the amount of the compound in the pharmaceutical composition is about 3 mg.

26. The pharmaceutical composition of claim 21, wherein the amount of the compound in the pharmaceutical composition is about 6 mg.

27. The pharmaceutical composition of claim 21, wherein the amount of the compound in the pharmaceutical composition is about 10 mg.

28. The pharmaceutical composition of any one of claims 21 to 27, further comprising an ionic surfactant.

29. The pharmaceutical composition of claim 28, wherein the ionic surfactant is sodium lauryl sulfate.

30. A pharmaceutical composition comprising, a compound of formula: 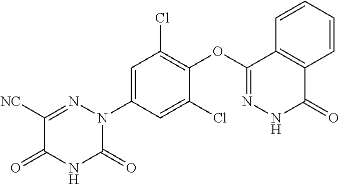 [SMILES: N#Cc1nn(-c2cc(Cl)c(Oc3n[nH]c(=O)c4ccccc34)c(Cl)c2)c(=O)[nH]c1=O]
or a pharmaceutically acceptable salt thereof and sodium lauryl sulfate.

31. A crystalline potassium salt of a compound of formula: 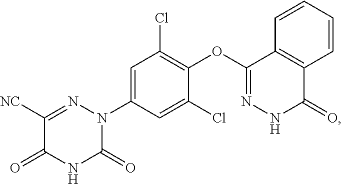 [SMILES: N#Cc1nn(-c2cc(Cl)c(Oc3n[nH]c(=O)c4ccccc34)c(Cl)c2)c(=O)[nH]c1=O]
having an XRPD spectrum substantially as shown in FIG. 16.

32. A crystalline potassium salt of a compound of formula: 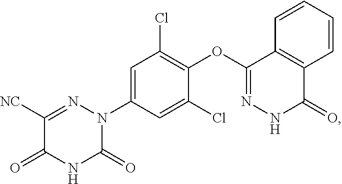 [SMILES: N#Cc1nn(-c2cc(Cl)c(Oc3n[nH]c(=O)c4ccccc34)c(Cl)c2)c(=O)[nH]c1=O]
having an XRPD spectrum comprising peaks at angles 2-theta of 6.78±0.20, 11.35±0.20, and 20.51±0.20 degrees.

33. A crystalline sodium salt of a compound of formula: 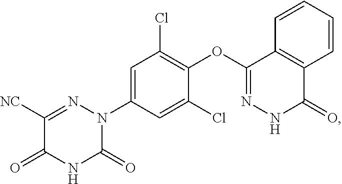 [SMILES: N#Cc1nn(-c2cc(Cl)c(Oc3n[nH]c(=O)c4ccccc34)c(Cl)c2)c(=O)[nH]c1=O]
having an XRPD spectrum substantially as shown in FIG. 18.

34. A crystalline sodium salt of a compound of formula: 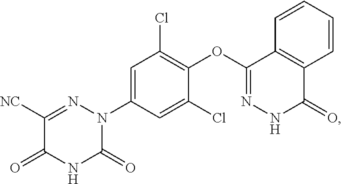 [SMILES: N#Cc1nn(-c2cc(Cl)c(Oc3n[nH]c(=O)c4ccccc34)c(Cl)c2)c(=O)[nH]c1=O]
having an XRPD spectrum comprising peaks at angles 2-theta of 5.51±0.20, 8.47±0.20, and 16.57±0.20 degrees.
